SEQUENCE LISTING (40 sequences)

SEQ ID NO: 1 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSNAGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTDGENNNSDFSWTGATKYHLNGRDSLVNPGPAMASHKDDEEKFFPQSGVLIFGKQSAAGADVAIDSVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 2 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSNAGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKVDGENNNSDFSWTGATKYHLNGRDSLVNPGPAMASHKDDEEKFFPQSGVLIFGKQSAAGADVAIDSVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 3 - DNA, 283 bp, Gallus gallus
  source 1..283: mol_type = genomic DNA
catggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - protein, 11 aa, synthetic construct
SAAGADXAXDS

SEQ ID NO: 5 - skipped

SEQ ID NO: 6 - protein, 736 aa, unidentified
  source 1..736: note = Adeno associated virus serotype 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - skipped

SEQ ID NO: 8 - DNA, 687 bp, synthetic construct
  source 1..687: mol_type = other DNA
atgtggactctcgggcgccgcgcagtagccggcctcctggcgtcacccagcccagcccaggcccagaccctcacccgggtcccgcggccggcagagttggccccactctgcggccgacgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgaaccggactgttaccaacacccacacccctgtgatgaaacaaaacccataaatgc

SEQ ID NO: 9 - DNA, 1796 bp, Homo sapiens
  source 1..1796: mol_type = genomic DNA
tccccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttg

SEQ ID NO: 10-11 - skipped (2 entries)

SEQ ID NO: 12 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 13 - skipped

SEQ ID NO: 14 - protein, 219 aa, synthetic construct
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDAYPYDVPDYA

SEQ ID NO: 15 - DNA, 587 bp, synthetic construct
  source 1..587: mol_type = other DNA
tcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 16 - DNA, 3579 bp, synthetic construct
  source 1..3579: mol_type = other DNA
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcaacgcgtctcgaggcggccgccaccatgtggactctcgggcgccgcgcagtagccggcctcctggcgtcacccagcccagcccaggcccagaccctcacccgggtcccgcggccggcagagttggccccactctgcggccgacgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttacccttacgatgtaccggattatgcatgagtcgacgcggccgcgcggatccaaaaagcttggggctagcaaaatgcatggatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggcc

SEQ ID NO: 17 - DNA, 2529 bp, synthetic construct
  source 1..2529: mol_type = other DNA
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtaccgatctaggcctactagtctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaattcactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtggactctcgggcgccgcgcagtagccggcctcctggcgtcacccagcccagcccaggcccagaccctcacccgggtcccgcggccggcagagttggccccactctgcggccgacgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttacccttacgatgtaccggattatgcatgagtcgacgcggccgcgcggatccaaaaagcttggggctagcaaaatgcatggatcctcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggcc

SEQ ID NO: 18 - DNA, 633 bp, synthetic construct
  source 1..633: mol_type = other DNA
atgtggactctcgggcgccgcgcagtagccggcctcctggcgtcacccagcccagcccaggcccagaccctcacccgggtcccgcggccggcagagttggccccactctgcggccgacgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttga

SEQ ID NO: 19 - DNA, 216 bp, synthetic construct
  source 1..216: mol_type = other DNA
agcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcgataaggatc

SEQ ID NO: 20 - DNA, 54 bp, Gallus gallus
  source 1..54: mol_type = genomic DNA
accggactgttaccaacacccacacccctgtgatgaaacaaaacccataaatgc

SEQ ID NO: 21 - DNA, 723 bp, Mus musculus
  source 1..723: mol_type = genomic DNA
gttccccaggccaagcaggttagggaaagctgagatgaaaggagggggtgggggggcttaatctgtgaaaataccttggcagttgaaggaaggagaatggtcttaggtttgtgctgggtctctggtgctcttcactgttgcctgtcactttttttctctttttgtaataccgataacatcaatgtaacacttgagatctttctgaactcctgttgtaatggctaaaatcacataaacctttgtgtcctaacggtgtcctcttttctttctcttcctttctccctatcaagctctttgttatcaacttaaatccacctttctgaacacagaaaattttcttcctttagaaaagactgaaagctcaggtgtttgtttcttctttgggtatgctattaatataagttgaaccaaaaatggccttactcaatccaacaatgagaagaaacaatggattttaagatgtcctttggtacacgacttgtttattgagagtgtgtttttatgaagatgttgccagacctttatttccttaaaggtttatttgagcagttacttagaggcacacagcacagaatttaaaggagagtgaaaaattagtttcctcttctgtgtgctttgatccagccttcctccctgaagaatagcattatcaataatgttgatcattgatagtagtcaccaagttatatcatttttatgatgctgggaatcaaacccagggcc

SEQ ID NO: 22 - DNA, 1033 bp, Mus musculus
  source 1..1033: mol_type = genomic DNA
ccaggacgaggtgaaagctgagaccatccgcagcctgaggaagtctttcgccagcctcttctccgactgacatcccactctgagaacccctaaacccctagacagccctctctgggtcctgagtccatttctcacttttggaacctccaaatcccttgagaacccctctcctggttctcctagagcccacttctcattcctcagtatgtcccttgagaaacctggtcctaaatccagttctcacatttgggaatccccaagtccatttagaatcctgttcctggtcacctccagatctgtttgagaacctccaaactcctgaagacctctacttctggtacatctaaagggtgatttcttccctgaaagttcctgaatggcagaaaacctgtttctgacctaatacatcgaatcctctcctggagctctgaaattcctgaaaaaccctattctactcttggtcctgaatctctccaacacaccttgctcctaatcagaaatttccaattccataagaagcccctatccttaaacctcttccataggtcatctctctggagatccacatcttcaaatgccacctaccatccaccaagcagtccctcaaggacttcccttcacctcttcctctccatcccattcaatatactggaagttccttccacttctaggacccctaagttccctcctccagaaacccctccccaatttcctgcctctgacagctgtctttaaatatgcaaactccacccatcctcagaatcctttgcacaaggaaggcctgtggtctcccactcccctacctttgccaatgtgtttatctgtgactgacttggcctccttttgtgccatgcttggcatatgtggtcctcattcatgccgcctgtggtaatgcgtacagtggcactgtttttgtggtctgcatcttcctccaacctccactccttgcctggacttgccccacccctcagcggctctgaacccccaagagaagagtcgggaagcaaaataaacaagcaaaggcccagcagaa

SEQ ID NO: 23 - DNA, 412 bp, Mus musculus
  source 1..412: mol_type = genomic DNA
aacacatatcagatgctgttatatatgtgtagtggctattcctggttgtcaacttgacaatatttggaatgaactacaatccggaagccctgtaagtcgttgaaatgtaaatagatttttcttctaaattaaatacttttctgtggttttattattacgtatgaggtatgtatttggaatacacaaacatatttttaacagtttatgctcataatccaccatgacttgtaaagggtgaatattataaaccttatatatctacaactacaggcctaactttcggttggtccgagagtagtggtgtttacaactaattgatcacaaccagttacagatttctttgttccttctccgctcccactgcttcacttgaccagccttttgttgaatgagctattaacattccctcctg

SEQ ID NO: 24 - DNA, 1666 bp, synthetic construct
  source 1..1666: mol_type = other DNA
atgtggactctcgggcgccgcgcagtagccggcctcctggcgtcacccagcccagcccaggcccagaccctcacccgggtcccgcggccggcagagttggccccactctgcggccgacgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgaccaggacgaggtgaaagctgagaccatccgcagcctgaggaagtctttcgccagcctcttctccgactgacatcccactctgagaacccctaaacccctagacagccctctctgggtcctgagtccatttctcacttttggaacctccaaatcccttgagaacccctctcctggttctcctagagcccacttctcattcctcagtatgtcccttgagaaacctggtcctaaatccagttctcacatttgggaatccccaagtccatttagaatcctgttcctggtcacctccagatctgtttgagaacctccaaactcctgaagacctctacttctggtacatctaaagggtgatttcttccctgaaagttcctgaatggcagaaaacctgtttctgacctaatacatcgaatcctctcctggagctctgaaattcctgaaaaaccctattctactcttggtcctgaatctctccaacacaccttgctcctaatcagaaatttccaattccataagaagcccctatccttaaacctcttccataggtcatctctctggagatccacatcttcaaatgccacctaccatccaccaagcagtccctcaaggacttcccttcacctcttcctctccatcccattcaatatactggaagttccttccacttctaggacccctaagttccctcctccagaaacccctccccaatttcctgcctctgacagctgtctttaaatatgcaaactccacccatcctcagaatcctttgcacaaggaaggcctgtggtctcccactcccctacctttgccaatgtgtttatctgtgactgacttggcctccttttgtgccatgcttggcatatgtggtcctcattcatgccgcctgtggtaatgcgtacagtggcactgtttttgtggtctgcatcttcctccaacctccactccttgcctggacttgccccacccctcagcggctctgaacccccaagagaagagtcgggaagcaaaataaacaagcaaaggcccagcagaa

SEQ ID NO: 25 - DNA, 1356 bp, synthetic construct
  source 1..1356: mol_type = other DNA
atgtggactctcgggcgccgcgcagtagccggcctcctggcgtcacccagcccagcccaggcccagaccctcacccgggtcccgcggccggcagagttggccccactctgcggccgacgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgagttccccaggccaagcaggttagggaaagctgagatgaaaggagggggtgggggggcttaatctgtgaaaataccttggcagttgaaggaaggagaatggtcttaggtttgtgctgggtctctggtgctcttcactgttgcctgtcactttttttctctttttgtaataccgataacatcaatgtaacacttgagatctttctgaactcctgttgtaatggctaaaatcacataaacctttgtgtcctaacggtgtcctcttttctttctcttcctttctccctatcaagctctttgttatcaacttaaatccacctttctgaacacagaaaattttcttcctttagaaaagactgaaagctcaggtgtttgtttcttctttgggtatgctattaatataagttgaaccaaaaatggccttactcaatccaacaatgagaagaaacaatggattttaagatgtcctttggtacacgacttgtttattgagagtgtgtttttatgaagatgttgccagacctttatttccttaaaggtttatttgagcagttacttagaggcacacagcacagaatttaaaggagagtgaaaaattagtttcctcttctgtgtgctttgatccagccttcctccctgaagaatagcattatcaataatgttgatcattgatagtagtcaccaagttatatcatttttatgatgctgggaatcaaacccagggcc

SEQ ID NO: 26 - DNA, 1045 bp, synthetic construct
  source 1..1045: mol_type = other DNA
atgtggactctcgggcgccgcgcagtagccggcctcctggcgtcacccagcccagcccaggcccagaccctcacccgggtcccgcggccggcagagttggccccactctgcggccgacgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgaaacacatatcagatgctgttatatatgtgtagtggctattcctggttgtcaacttgacaatatttggaatgaactacaatccggaagccctgtaagtcgttgaaatgtaaatagatttttcttctaaattaaatacttttctgtggttttattattacgtatgaggtatgtatttggaatacacaaacatatttttaacagtttatgctcataatccaccatgacttgtaaagggtgaatattataaaccttatatatctacaactacaggcctaactttcggttggtccgagagtagtggtgtttacaactaattgatcacaaccagttacagatttctttgttccttctccgctcccactgcttcacttgaccagccttttgttgaatgagctattaacattccctcctg

SEQ ID NO: 27 - DNA, 2429 bp, synthetic construct
  source 1..2429: mol_type = other DNA
atgtggactctcgggcgccgcgcagtagccggcctcctggcgtcacccagcccagcccaggcccagaccctcacccgggtcccgcggccggcagagttggccccactctgcggccgacgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgcttgatccccagccccgttttaaggacattaaaagctatcaggccaagaccccagcttcattatgcagctgaggtctgttttttgttgttgttgttgtttattttttttattcctgcttttgaggacagttgggctatgtgtcacagctctgtagaaagaatgtgttgcctcctaccttgcccccaagttctgatttttaatttctatggaagattttttggattgtcggatttcctccctcacatgataccccttatcttttataatgtcttatgcctatacctgaatataacaacctttaaaaaagcaaaataataagaaggaaaaattccaggagggaaaatgaattgtcttcactcttcattctttgaaggatttactgcaagaagtacatgaagagcagctggtcaacctgctcactgttctatctccaaatgagacacattaaagggtagcctacaaatgttttcaggcttctttcaaagtgtaagcacttctgagctctttagcattgaagtgtcgaaagcaactcacacgggaagatcatttcttatttgtgctctgtgactgccaaggtgtggcctgcactgggttgtccagggagacctagtgctgtttctcccacatattcacatacgtgtctgtgtgtatatatattttttcaatttaaaggttagtatggaatcagctgctacaagaatgcaaaaaatcttccaaagacaagaaaagaggaaaaaaagccgttttcatgagctgagtgatgtagcgtaacaaacaaaatcatggagctgaggaggtgccttgtaaacatgaaggggcagataaaggaaggagatactcatgttgataaagagagccctggtcctagacatagttcagccacaaagtagttgtccctttgtggacaagtttcccaaattccctggacctctgcttccccatctgttaaatgagagaatagagtatggttgattcccagcattcagtggtcctgtcaagcaacctaacaggctagttctaattccctattgggtagatgaggggatgacaaagaacagtttttaagctatataggaaacattgttattggtgttgccctatcgtgatttcagttgaattcatgtgaaaataatagccatccttggcctggcgcggtggctcacacctgtaatcccagcacttttggaggccaaggtgggtggatcacctgaggtcaggagttcaagaccagcctggccaacatgatgaaaccccgtctctactaaaaatacaaaaaattagccgggcatgatggcaggtgcctgtaatcccagctacttgggaggctgaagcggaagaatcgcttgaacccagaggtggaggttgcagtgagccgagatcgtgccattgcactgtaacctgggtgactgagcaaaactctgtctcaaaataataataacaatataataataataatagccatcctttattgtacccttactgggttaatcgtattataccacattacctcattttaatttttactgacctgcactttatacaaagcaacaagcctccaggacattaaaattcatgcaaagttatgctcatgttatattattttcttacttaaagaaggatttattagtggctgggcatggtggcgtgcacctgtaatcccaggtactcaggaggctgagacgggagaattgcttgaccccaggcggaggaggttacagtgagtcgagatcgtacctgagcgacagagcgagactccgtctcaaaaaaaaaaaaaaggagggtttattaatgagaagtttg

SEQ ID NO: 28 - DNA, 952 bp, synthetic construct
  source 1..952: mol_type = other DNA
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 29 - DNA, 143 bp, unidentified
  source 1..143: mol_type = genomic DNA; note = Adeno associated virus serotype 2
ggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 30 - DNA, 143 bp, unidentified
  source 1..143: mol_type = genomic DNA; note = Adeno associated virus serotype 2
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggcc

SEQ ID NO: 31 - DNA, 490 bp, Homo sapiens
  source 1..490: mol_type = genomic DNA
gatctaggcctactagtctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcga

SEQ ID NO: 32 - protein, 735 aa, unidentified [sequence omitted]

SEQ ID NO: 33 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVTTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 34 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTDGENNNSDFSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 35 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSTTPADNNNSDFSWTGATKYHLNGRDSLVNPGPAMASHKDDDEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 36 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSNAGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTASGNVTQSTLQFSQAGASDIRDQSRNWLPGPCYRQQRVSKTSADNNNSEYSWTGATKYHLNGRDSLVNPGPAMASHRDDDDKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 37 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQGVSKVSADNNNSEFSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 38 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTVTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 39 - protein, 724 aa, unidentified
  source 1..724: note = Adeno associated virus serotype 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 40 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMASQARNWVPGPSYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

(19) United States
(12) Patent Application Publication
(10) Pub. No.: US 2024/0415983 A1
(43) Pub. Date: Dec. 19, 2024

(54) AAV-MEDIATED THERAPIES FOR VISION LOSS ASSOCIATED WITH FRIEDREICH’S ATAXIA

(71) Applicant: University of Florida Research Foundation, Incorporated, Gainesville, FL (US)

(72) Inventors: Sanford L. Boye, Gainesville, FL (US); Shannon E. Boye, Gainesville, FL (US)

(73) Assignee: University of Florida Research Foundation, Incorporated, Gainesville, FL (US)

(21) Appl. No.: 18/706,176

(22) PCT Filed: Oct. 31, 2022
(86) PCT No.: PCT/US2022/078984

§ 371 Date: Apr. 30, 2024

Related U.S. Application Data
(60) Provisional application No. 63/285,988, filed on Dec. 3, 2021.
(60) Provisional application No. 63/274,037, filed on Nov. 1, 2021.

(51) Int. Cl. A61K 48/00; A61P 21/00; C07K 14/47; C12N 15/86

(52) U.S. Cl. CPC A61K 48/0058; A61K 48/0075; A61P 21/00; C07K 14/47; C12N 15/86; C12N 2750/14143; C12N 2750/14171; C12N 2800/22

(57) ABSTRACT

The present disclosure provides for recombinant AAV therapies for the treatment of vision loss associated with Friedreich's ataxia. These therapies are designed for administration to subjects, such as human subjects, including humans diagnosed with or suffering from Friedreich's ataxia. The disclosed vectors comprise transgenes that encode frataxin protein, such as a FXN trans gene encoding human frataxin protein.

### CROSS-REFERENCE TO RELATED APPLICATIONS

This application claims the benefit of the filing dates of U.S. Provisional Application No. 63/274,037, filed Nov. 1, 2021, and U.S. Provisional Application No. 63/285,988, filed Dec. 3, 2021, the entire contents of each of which are incorporated by reference.

### GOVERNMENT SUPPORT

This invention was made with government support under grant number R01 EY024280, awarded by the National Institutes of Health. The government has certain rights in the invention.

### NON-FEDERAL SUPPORT

This invention was made in whole or in part from funding received under contract number AGR00019220, received from Friedreich's Ataxia Research Alliance.

### REFERENCE TO AN ELECTRONIC SEQUENCE LISTING

The contents of the electronic sequence listing (U120270076WO00-SEQ-EPG.xml; Size: 63,102 bytes; and Date of Creation: Oct. 24, 2022) is herein incorporated by reference in its entirety.

### BACKGROUND OF THE INVENTION

Friedreich's Ataxia (FA, or FRDA) is the leading form of hereditary ataxia among Caucasians and is caused by autosomal recessive mutations in the frataxin (FXN) gene, which encodes the mitochondrial protein frataxin. Repeat expansions of the trinucleotide GAA in the FXN gene leads to lower levels of functional frataxin. This causes an abnormal influx of iron into mitochondria, an efflux of iron from the cytoplasm, and damage to the nervous system. FA primarily presents in degeneration of the spinal cord and the peripheral nerves that connect the spinal cord to the body's muscles and sensory organs. FA affects the function of the cerebellum and also the musculature of the heart. FA patients typically present before the second decade with loss of muscular function, speech impediments, and cardiomyopathy. In addition to these peripheral complications, defects of the central nervous system including hearing and vision loss ultimately manifest. FA affects 1 in 50,000 people worldwide. Symptoms generally present at puberty and patients have a shorter than normal life expectancy reaching 40-50 years of age. At present writing, patients with FA receive palliative care, as there is no FDA-approved treatment for this disease.

### SUMMARY OF THE INVENTION

The present disclosure provides rAAV therapies for the treatment of vision loss symptoms of FA. These therapies are designed for administration to the eyes of subjects, such as human subjects, including humans diagnosed with or suffering from FA. The disclosed rAAV vectors may provide for improved retinal structure and function after administration to subjects. The disclosed vectors may provide for amelioration or reversal, e.g., a partial or complete reversal, of the symptoms of FA, including loss of retinal ganglion cells, thinning of the nerve fiber layer, optic nerve atrophy, nystagmus, and loss of visual field.

Accordingly, provided herein are retina-targeted, AAV-based therapies for preserving and restoring vision in FA patients. These therapies may be administered alone in advanced-stage patients, or in combination with systemic/intrathecal treatments, such as systemic AAV-FXN treatments, in those patients with less advanced disease.

The disclosed vectors comprise transgenes that encode frataxin protein, such as a FXN transgene encoding human frataxin protein. In some embodiments, the vector and/or human FXN transgene (or heterologous nucleic acid) of the disclosed vectors comprise a modified 3′ untranslated region, or UTR. In some embodiments, the vector and/or FXN transgene of the disclosed vectors comprises a 3′ untranslated region comprising one or more axon targeting motifs (ATMs). The 3′ UTR or ATM may be positioned after (3′ to) the FXN coding sequence. ATMs are small cis-acting elements located in the 3′ UTR that enhance trafficking (i.e., transport) of the FXN-encoding mRNA to distal axons of RGCs in the retina. In some embodiments of the disclosed vectors, any of these ATMs may comprise sequences derived from one or more of β-actin, synapsin, β-tubulin, and non-coding RNA Y3. In some embodiments, the ATMs of the vector are derived from β-actin or synapsin.

The present disclosure is based in part on the incorporation of ATMs designed for axon targeting for improved ocular therapies for FA.

Progress has been made to develop a systemic gene therapy to treat FA. While systemically-delivered Adeno associated viral (AAV) vectors have been generated that can efficiently target the muscle and heart, and intrathecal injections can efficiently transduce the brain, neither will mediate sufficient levels of therapeutic transgene in the retina to prevent vision loss. A more directed approach is needed to treat the ocular phenotype of this disease, which includes loss of retinal ganglion cells (RGCs), thinning of the nerve fiber layer, optic nerve atrophy, nystagmus (an involuntary, rapid and repetitive movement of the eye), and loss of visual field. Thus, there is a deficiency in the art for an AAV vector that can efficiently deliver a therapeutic nucleic acid payload to the eye of a subject or patient for treatment of ocular phenotypes of FA.

The present disclosure also provides for the use of RGC-targeting promoters, such as the human synapsin 1 promoter, and of AAV capsids that display improved retinal transduction efficiency and that exhibits enhanced lateral spread after subretinal injection. Additional non-limiting RGC-targeting promoters (or “RGC promoters”) include Nefh(Ple345) (e.g., human and mouse), Sncg (e.g., human and mouse), Cx36 (e.g., human), and dcx (e.g., human). In addition to subretinal injection, other methods of delivery are contemplated in this disclosure. These methods include intravitreal and subILM injection.

Accordingly, the present disclosure provides nucleic acid vectors comprising a synthetic human FXN transgene sequence operably controlled by a promoter, such as a human synapsin 1 (hSYN1) promoter or another RGC specific promoter, that may be encapsidated into a viral particle, such as an AAV particle. The hSYN1 promoter may be used to mediate efficient expression in non-human primate RGCs. Additional non-limiting exemplary RGC promoters include Nefh(Ple345) (e.g., human and mouse), Sncg (e.g., human and mouse), Cx36 (e.g., human), and dcx (e.g., human). The disclosed nucleic acid vectors may comprise AAV inverted terminal repeats flanking the polynucleotide comprising the FXN transgene.

Frataxin is a highly conserved, 210 amino acid (˜17 kDa) protein. While frataxin's specific function remains unclear, homozygous deletions are embryonically lethal. Evidence suggests frataxin is involved in iron metabolism, iron storage, iron-sulfur cluster (ISC) formation, and protection against reactive oxygen species (ROS). Excess mitochondrial iron increases the incidence of iron-catalyzed reduction of hydrogen peroxide-generating ROS. The increase in ROS disrupts iron homeostasis in the mitochondria and affects the ISC aconitase, a major component of cellular respiration.

Major advances in the field of gene therapy have been achieved by using viruses to deliver therapeutic genetic material. The AAV vector has attracted considerable attention as a highly effective viral vector for gene therapy due to its low immunogenicity and ability to effectively transduce non-dividing cells. AAV has been shown to infect a variety of cell and tissue types, and significant progress has been made over the last decade to adapt this viral system for use in human gene therapy. Recombinant adeno-associated virus (rAAV) vectors have been used successfully for in vivo gene transfer in numerous pre-clinical animal models of human disease and have been used successfully for long-term expression of a wide variety of therapeutic genes. AAV vectors have also generated long-term clinical benefit in humans when targeted to immune-privileged sites, e.g., ocular delivery for Leber's congenital amaurosis. A major advantage of this vector is its comparatively low immune profile, eliciting only limited inflammatory responses and, in some cases, even directing immune tolerance to transgene products. Nonetheless, the therapeutic efficiency, when targeted to non-immune privileged organs, has been limited in humans due to antibody and CD8+ T cell responses against the viral capsid, while in animal models, adaptive responses to the transgene product have also been reported.

AAV has become the vector of choice for targeting therapeutic genes to the retina. Both naturally occurring and synthetic AAVs have been identified that display retinal tropism. The AAV capsid variants P2-V1 and P2-V1(Y-F+T-V) were chosen as exemplary capsids for their ability to transduce retinal ganglion cells (RGCs) and avoid neutralization by AAV2 NAbs. In particular, antibody neutralization studies suggest a lower frequency of neutralizing antibodies to P2-V1(Y-F+T-V) compared with AAV2.

Recently, AAV therapies for systemic delivery of functional frataxin have been generated. These therapies have been adapted for intrathecal, intravenous, intramuscular, and intraspinal delivery. These therapies are designed to treat muscular and peripheral nervous system phenotypes of FA, such as muscle weakness, ataxia, a loss of balance and coordination. Examples of such therapies include AAV9-CBA-FXN.

The inventors have applied novel and existing AAV capsid variants and wild-type capsids to the delivery of a frataxin-encoding FXN heterologous nucleic acid to subjects. The present disclosure is based, at least in part, on the addition of an ATM into rAAV vectors comprising the human FXN heterologous nucleic acid. For instance, the ATM may be added to the 3′ end of the FXN coding sequence. The human FXN heterologous nucleic acids of the disclosure may also comprise one or more silent mutations in the coding region (i.e., that do not result in mutations in the encoded protein sequence). In some embodiments, the FXN transgene of any of the disclosed rAAV vectors has been codon-optimized for human expression. A non-limiting example FXN heterologous nucleic acid comprises a sequence at least 80%, 85%, 90%, 91%, 92%, 93%, 94%, 95%, 96%, 97%, 98%, 99%, or 100% identical to the sequence set forth as SEQ ID NO: 18: ATGTGGACTCTCGGGCGCCGCGCAGTAGCCGGCCTCCTGGCGTCACCCAGCCCAGCCCA GGCCCAGACCCTCACCCGGGTCCCGCGGCCGGCAGAGTTGGCCCCACTCTGCGGCCGAC GTGGCCTGCGCACCGACATCGATGCGACCTGCACGCCCCGCCGCGCAAGTTCGAACCAA CGTGGCCTCAACCAGATTTGGAATGTCAAAAAGCAGAGTGTCTATTTGATGAATTTGAGG AAATCTGGAACTTTGGGCCACCCAGGCTCTCTAGATGAGACCACCTATGAAAGACTAGC AGAGGAAACGCTGGACTCTTTAGCAGAGTTTTTTGAAGACCTTGCAGACAAGCCATACA CGTTTGAGGACTATGATGTCTCCTTTGGGAGTGGTGTCTTAACTGTCAAACTGGGTGGAG ATCTAGGAACCTATGTGATCAACAAGCAGACGCCAAACAAGCAAATCTGGCTATCTTCTC CATCCAGTGGACCTAAGCGTTATGACTGGACTGGGAAAAACTGGGTGTACTCCCACGAC GGCGTGTCCCTCCATGAGCTGCTGGCCGCAGAGCTCACTAAAGCCTTAAAAACCAAACT GGACTTGTCTTCCTTGGCCTATTCCGGAAAAGATGCTTGA (SEQ ID NO: 18), which has a C->A substitution as compared to NCBI reference NM_000144.5 to remove a NotI recognition sequence. In some cases, the FXN heterologous nucleic acid comprises a sequence at least 80% identical to NCBI reference NM_000144.5.

In some aspects, subretinal administration of the disclosed rAAV vectors is provided. Subretinal injection of AAV is commonly used when transgene expression is required in the RGCs, the retinal pigment epithelium (RPE), the photoreceptors (PR), and/or other retinal cells. The subretinal injection creates a temporary bullous detachment, separating the photoreceptor outer segments from the RPE layer. Typically the subretinal injection bleb resolves over the following few days in subjects. Subretinal injection likely has some deleterious effects on the photoreceptors, with such effects conceivably being more severe in a retina already compromised by disease. However, administration into the vitreous (intravitreal injection) presents its own challenges, including an observed dilution effect, the presence of the inner limiting membrane (ILM) barrier, and severe inflammation owing to the increased biodistribution of AAV to the peripheral circulation when delivered via this route compared to when delivered to the subretinal space. Thus, there remains a need in the art to administer gene therapies to the retinas of FA patients subretinally while minimizing adverse effects such as retinal detachment.

Accordingly, in some aspects, the disclosure provides methods for expressing a human FXN nucleic acid segment in one or more photoreceptor cells of a mammalian subject, wherein the method comprises subretinally or intravitreally administering to one or both eyes of the subject an rAAV particle as described herein. In particular embodiments, administration is by subretinal injection. In some embodiments, administration does not occur by intravitreal injection.

In some aspects, the disclosure provides recombinant AAV (rAAV) vectors that comprise a polynucleotide that comprises a heterologous nucleic acid encoding a frataxin protein (e.g., a human frataxin protein), 3′ UTR, and regulatory elements. In some embodiments, the regulatory elements may comprise a WPRE element, an SV40 intron, a polyadenylation signal sequence, and/or a combination thereof. In various embodiments, the rAAV vector comprises a 3′ untranslated region (positioned 3′ of the frataxin-encoding heterologous nucleic acid) that comprises one or more ATMs. In some embodiments, the rAAV vector comprises one or more ATMs. In some embodiments, the 3′ UTR and/or rAAV vector comprises 1, 2, 3, 4, 5, or more than 5 ATMs. In some embodiments, the 3′ UTR and/or rAAV vector comprises between 2 and 5, between 1 and 4, between 1 and 3, between 2 and 4, or between 2 and 3 ATMs. In some embodiments, the 3′ UTR and/or rAAV vector comprises a 3′ UTR from β-actin. In some embodiments, the 3′ UTR and/or rAAV vector comprises an ATM from β-actin. In some embodiments, the 3′ UTR and/or rAAV vector comprises a 3′ UTR from synapsin. In some embodiments, the 3′ UTR and/or rAAV vector comprises an ATM from synapsin. In some embodiments, the 3′ UTR and/or rAAV vector comprises a 3′ UTR from β-tubulin. In some embodiments, the 3′ UTR and/or rAAV vector comprises an ATM from β-tubulin. In some embodiments, the 3′ UTR and/or rAAV vector comprises a 3′ UTR from non-coding RNA Y3. In some embodiments, the 3′ UTR and/or rAAV vector comprises a 3′ UTR sequence from β-actin from Gallus gallus, e.g., ACCGGACTGTTACCAACACCCACACCCCTGTGATGAAACAAAACCCATAAATGC (SEQ ID NO: 20). In some embodiments, the 3′ UTR and/or rAAV vector comprises a 3′ UTR sequence at least 80%, 85%, 90%, 91%, 92%, 93%, 94%, 95%, 96%, 97%, 98%, 99%, or 100% identical to SEQ ID NO: 20. In some embodiments, the 3′ UTR and/or rAAV vector comprises a 3′ UTR sequence from the Tubb2b (mouse) gene, e.g., GTTCCCCAGGCCAAGCAGGTTAGGGAAAGCTGAGATGAAAGGAGGGGGTGGGGGGGCT TAATCTGTGAAAATACCTTGGCAGTTGAAGGAAGGAGAATGGTCTTAGGTTTGTGCTGGG TCTCTGGTGCTCTTCACTGTTGCCTGTCACTTTTTTTCTCTTTTTGTAATACCGATAACATC AATGTAACACTTGAGATCTTTCTGAACTCCTGTTGTAATGGCTAAAATCACATAAACCTT TGTGTCCTAACGGTGTCCTCTTTTCTTTCTCTTCCTTTCTCCCTATCAAGCTCTTTGTTATC AACTTAAATCCACCTTTCTGAACACAGAAAATTTTCTTCCTTTAGAAAAGACTGAAAGCT CAGGTGTTTGTTTCTTCTTTGGGTATGCTATTAATATAAGTTGAACCAAAAATGGCCTTAC TCAATCCAACAATGAGAAGAAACAATGGATTTTAAGATGTCCTTTGGTACACGACTTGTT TATTGAGAGTGTGTTTTTATGAAGATGTTGCCAGACCTTTATTTCCTTAAAGGTTTATTTG AGCAGTTACTTAGAGGCACACAGCACAGAATTTAAAGGAGAGTGAAAAATTAGTTTCCT CTTCTGTGTGCTTTGATCCAGCCTTCCTCCCTGAAGAATAGCATTATCAATAATGTTGATC ATTGATAGTAGTCACCAAGTTATATCATTTTTATGATGCTGGGAATCAAACCCAGGGCC (SEQ ID NO: 21). In some embodiments, the 3′ UTR and/or rAAV vector comprises a 3′ UTR sequence at least 80%, 85%, 90%, 91%, 92%, 93%, 94%, 95%, 96%, 97%, 98%, 99%, or 100% identical to SEQ ID NO: 21. In some embodiments, the 3′ UTR and/or rAAV vector comprises a 3′ UTR sequence from synl (synapsin, mouse), e.g., CCAGGACGAGGTGAAAGCTGAGACCATCCGCAGCCTGAGGAAGTCTTTCGCCAGCCTCT TCTCCGACTGACATCCCACTCTGAGAACCCCTAAACCCCTAGACAGCCCTCTCTGGGTCC TGAGTCCATTTCTCACTTTTGGAACCTCCAAATCCCTTGAGAACCCCTCTCCTGGTTCTCC TAGAGCCCACTTCTCATTCCTCAGTATGTCCCTTGAGAAACCTGGTCCTAAATCCAGTTCT CACATTTGGGAATCCCCAAGTCCATTTAGAATCCTGTTCCTGGTCACCTCCAGATCTGTTT GAGAACCTCCAAACTCCTGAAGACCTCTACTTCTGGTACATCTAAAGGGTGATTTCTTCC CTGAAAGTTCCTGAATGGCAGAAAACCTGTTTCTGACCTAATACATCGAATCCTCTCCTG GAGCTCTGAAATTCCTGAAAAACCCTATTCTACTCTTGGTCCTGAATCTCTCCAACACAC CTTGCTCCTAATCAGAAATTTCCAATTCCATAAGAAGCCCCTATCCTTAAACCTCTTCCAT AGGTCATCTCTCTGGAGATCCACATCTTCAAATGCCACCTACCATCCACCAAGCAGTCCC TCAAGGACTTCCCTTCACCTCTTCCTCTCCATCCCATTCAATATACTGGAAGTTCCTTCCA CTTCTAGGACCCCTAAGTTCCCTCCTCCAGAAACCCCTCCCCAATTTCCTGCCTCTGACAG CTGTCTTTAAATATGCAAACTCCACCCATCCTCAGAATCCTTTGCACAAGGAAGGCCTGT GGTCTCCCACTCCCCTACCTTTGCCAATGTGTTTATCTGTGACTGACTTGGCCTCCTTTTGT GCCATGCTTGGCATATGTGGTCCTCATTCATGCCGCCTGTGGTAATGCGTACAGTGGCAC TGTTTTTGTGGTCTGCATCTTCCTCCAACCTCCACTCCTTGCCTGGACTTGCCCCACCCCTC AGCGGCTCTGAACCCCCAAGAGAAGAGTCGGGAAGCAAAATAAACAAGCAAAGGCCCA GCAGAA (SEQ ID NO: 22). In some embodiments, the 3′ UTR and/or rAAV vector comprises a 3′ UTR sequence at least 80%, 85%, 90%, 91%, 92%, 93%, 94%, 95%, 96%, 97%, 98%, 99%, or 100% identical to SEQ ID NO: 22. In some embodiments, the 3′ UTR and/or rAAV vector comprises a 3′ UTR sequence from Y3 non-coding RNA (ncRNA), e.g., AACACATATCAGATGCTGTTATATATGTGTAGTGGCTATTCCTGGTTGTCAACTTGACAA TATTTGGAATGAACTACAATCCGGAAGCCCTGTAAGTCGTTGAAATGTAAATAGATTTTT CTTCTAAATTAAATACTTTTCTGTGGTTTTATTATTACGTATGAGGTATGTATTTGGAATA CACAAACATATTTTTAACAGTTTATGCTCATAATCCACCATGACTTGTAAAGGGTGAATA TTATAAACCTTATATATCTACAACTACAGGCCTAACTTTCGGTTGGTCCGAGAGTAGTGG TGTTTACAACTAATTGATCACAACCAGTTACAGATTTCTTTGTTCCTTCTCCGCTCCCACT GCTTCACTTGACCAGCCTTTTGTTGAATGAGCTATTAACATTCCCTCCTG (SEQ ID NO: 23). In some embodiments, the 3′ UTR and/or rAAV vector comprises a 3′ UTR sequence from mouse Y3 ncRNA, e.g., U34826. In some embodiments, the 3′ UTR and/or rAAV vector comprises a 3′ UTR sequence at least 80%, 85%, 90%, 91%, 92%, 93%, 94%, 95%, 96%, 97%, 98%, 99%, or 100% identical to SEQ ID NO: 23.

The 3′ untranslated region of human frataxin may comprise, or consist of, the nucleic acid sequence of SEQ ID NO: 9. In some embodiments, the heterologous nucleic acid of the vector does not comprise the 5′ untranslated region (UTR) of the (wild-type) gene (or cDNA) encoding human frataxin. In some embodiments, the heterologous nucleic acid does not comprise the 3′ untranslated region of human frataxin. In some embodiments, the 3′ UTR of frataxin is replaced with a 3′ UTR derived from another gene. For instance, the 3′ UTR of frataxin may be replaced with the 3′ UTR of any of β-actin, synapsin, β-tubulin, or non-coding RNA Y3. In some embodiments, the heterologous nucleic acid does not comprise a cDNA derived from superoxide dismutase 1 or 2 genes (Sod1 or Sod2).

In some aspects, the rAAV vectors comprise a polynucleotide that comprises a heterologous nucleic acid comprising a sequence having at least 80% identity, at least 85% identity, at least 90% identity, at least 92% identity, at least 92.5% identity, at least 93% identity, at least 94% identity, at least 95% identity, 98% identity, or 99% identity to the nucleotide sequence of any one of SEQ ID NOS: 8 and 24-27. In some embodiments, the polynucleotide of comprises a heterologous nucleic acid comprising at least 92.5% identity to any one of SEQ ID NOS: 8 and 24-27. In particular embodiments, the polynucleotide comprises at least 98% identity to any one of SEQ ID NOS: 8 and 24-27. In some embodiments, the heterologous nucleic acid comprises a sequence that differs by 1, 2, 3, 4, 5, 10, 15, 20, 25, or more than 25 nucleotides from a nucleotide sequence of SEQ ID NOS: 8 and 24-27. In other embodiments, the heterologous nucleic acid comprises the sequence of any one of SEQ ID NOS: 8 and 24-27.

The polynucleotide may comprise a nucleic acid sequence encoding a hemagglutinin (HA) tag. In some embodiments, the polynucleotide comprises a promoter that is capable of expressing the heterologous nucleic acid in one or more photoreceptors or retinal pigment epithelial cells of a mammalian eye. The rAAV particle may comprise a polynucleotide comprising at least a first polynucleotide that comprises a retinal-specific or neuron-specific promoter operably linked to at least a first heterologous nucleic acid that encodes a therapeutic agent, for a time effective to produce the therapeutic agent in the one or more RGC cells of the mammal. In particular embodiments, the promoter is a synapsin promoter, such as a human synapsin 1 (hSYN1) promoter. Additional non-limiting exemplary RGC promoters include Nefh(Ple345) (e.g., human and mouse), Sncg (e.g., human and mouse), Cx36 (e.g., human), and dcx (e.g., human).

In some aspects, the disclosure provides rAAV particles comprising any of the rAAV vectors described herein. In some embodiments, the capsid encapsidating the rAAV vector in the rAAV particle comprises an AAV2(7m8) capsid, a DGE-DF capsid, a P2-V1 capsid, a P2-V2 capsid, a P2-V3 capsid, a P2-V1(Y-F) capsid, a P2-V1(Y-F+T-V) capsid, an AAV5 capsid or a variant thereof, an AAV2(4pMut)ΔHS capsid, an AAV8(Y447F+Y733F+T494V) capsid, or an AAV44.9(E531D) capsid. In some embodiments, the capsid is AAV5, or a variant thereof. Non-limiting example AAV capsid sequences are provided in Table 1. In some embodiments, the capsid is selected from any one of SEQ ID NOs: 1, 2, 6 and 32-40. In some embodiments, the capsid comprises an amino acid sequence having at least 85%, 90%, 95%, 97%, 98%, 99% identity to any one of SEQ ID NOs: 1, 2, 6 and 32-40. In particular embodiments, the capsid comprises an amino acid sequence having at least 95% identity to any of SEQ ID NOs: 1, 2, 6 and 32-40. The disclosure also provides compositions comprising any of the rAAV particles disclosed herein. The disclosure also provides cells (such as mammalian cells) comprising any of the rAAV particles disclosed herein.

In some aspects, the disclosure provides methods for transducing a mammalian photoreceptor cell or retinal pigment epithelium cell, the method comprising administering to one or both eyes of a mammal any of the rAAV particles or compositions of rAAV particles disclosed herein. Methods for treating or ameliorating FA in a mammal (such as a human), the method comprising administering to one or both eyes of the mammal any of the rAAV particles or compositions disclosed herein in an amount sufficient to treat or ameliorate the one or more symptoms of RGC degeneration, or other symptoms of FA, in the mammal are also provided. In some embodiments, the disclosed methods (or administration of any of the disclosed rAAV vectors) may arrest or reverse FA disease progression after onset.

In some embodiments, the disclosed methods comprise subretinal administration to a fovea of one or both eyes of the mammal. In particular embodiments, detachment of the fovea is minimized following subretinal administration. In some embodiments, any of the disclosed methods a) preserves one or more RGC cells, b) restores nerve fiber layer and/or optic nerve head structure, c) restores one or more rod- and/or cone-mediated functions, d) restores completely or partially visual behavior in one or both eyes, or e) any combination thereof. In particular embodiments, any of the disclosed methods restores, at least partially, integrity of the optic nerve head and/or the nerve fiber layer. In particular embodiments, any of the disclosed methods reverses, at least partially, RGC degeneration. In particular embodiments, any of the disclosed methods reverses, at least partially, loss of axons.

### BRIEF DESCRIPTION OF THE DRAWINGS

The following drawings form part of the present specification and are included to demonstrate certain aspects of the present invention. The invention may be better understood by reference to the following description taken in conjunction with the accompanying drawings, in which like reference numerals identify like elements, and in which:

FIGS. 1A-1B show transduction profile of novel AAV capsid variants mined via directed evolution in NHP following intravitreal injection (IVI) in Nrl-GFP mice. Onto certain variants (P2-V4 and P2-V1), additional Y-F capsid mutations were introduced via rational design. AAV-mediated mCherry expression in representative fluorescent fundus images (n=3 per capsid) taken at 4 weeks post-injection with 2×10⁹ vg (FIG. 1A). FACS analysis was used to quantify the percentage of cells that were GFP positive (rods), mCherry positive (neural retinal cells/glia transduced by rAAV) and both GFP and mCherry positive (rods transduced by rAAV), as shown in FIG. 1B. Error bars represent ±one standard deviation. Red bar represents the amount of transduction observed by benchmark vectors AAV2(7m8) (also known as AAV7m8) and AAV2(quadY-F+T-V). With the exception of P2-V4, all novel variants exhibited similar or higher potency than benchmark vectors in intravitreally-injected mice.

FIG. 2 shows fluorescent fundus photos taken of macaque retinas at 6 weeks post-intravitreal injection reveal that novel capsid variants outperform unmodified AAV2, and exhibit a clear dose response. Identical exposure and gain setting were used to capture all images. A summary of injections performed in each animal is provided in the insert table. All injected eyes are shown and were used in the quantification data presented in FIGS. 3A-3B.

FIGS. 3A-3B show the quantification of AAV-mediated GFP expression and intensity in foveal ring of intravitreally-injected macaques. Quantification was performed with the top row of high dose, and the top row of low dose fundus images in FIG. 2, respectively. Fundus quantification was performed by measuring the area and raw integrated density of foveal ring expression for each sample using the open-source Fiji software (see Schindelin et al., 2012). Briefly, each fundus image was converted into 8-bit grayscale, and the polygon selection tool was used to define the total region of foveal expression. Another region containing the foveal pit (area of low/no expression) was also defined and measured. The foveal ring measurements were then obtained by subtracting the area/intensity of the foveal pit from the total foveal region.

FIGS. 4A-4B show transduction of AAV2 and novel AAV capsid variant, P2-V1, in ARPE19 cells following incubation in human vitreous samples containing AAV2 NAbs. P2-V1 escapes neutralization in multiple human vitreous samples.

FIG. 5 shows an example of an AAV construct containing a promoter (e.g., synapsin, Nefh(Ple345), Sncg, Cx36, dcx), human frataxin, and an axon targeting motif (ATM). Exemplary ATMs are derived from the 3′ UTR of β-actin, synapsin, β-tubulin, or non-coding RNA Y3. Exemplary constructs may contain one or more of these ATMs positioned 3′ of the human FXN coding region.

FIG. 6 is a schematic showing both subretinal and intravitreal injection routes, and a detailed magnification of the human retina. The retina is separated from the vitreous by the inner limiting membrane (ILM) and contains three layers of cell bodies (retinal ganglion cell bodies in the RGC layer, bipolar cell bodies in the INL, and photoreceptor cell bodies in the ONL). Between the ONL and the RPE are the inner and outer segments of photoreceptors. FXN is expressed within RGCs and photoreceptor inner segments. Abbreviations: ILM—inner limiting membrane, RGC—retinal ganglion cells, IPL—inner plexiform layer, INL—inner nuclear layer, OPL—outer plexiform layer, ONL—outer nuclear layer, IS—inner segments, OLM—outer limiting membrane, OS—outer segments, RPE—retinal pigment epithelium.

FIG. 7 shows fundus images of WT mice intravitreally injected with 2×10⁹ vg of AAV containing self-complimentary smCBA-mCherry genomes. Images were acquired at 4 weeks post-injection with oue benchmark vector, AAV2(quadY-F+T-V), and four novel capsid.

FIG. 8 shows a fluorescent fundus image and retinal cryosection from macaque that received subILM injection of a total of 6.23×10⁹ vg of unmodified AAV2. RGCs are extensively transduced (right).

FIGS. 9A-9B show a six month natural history study of mRx-Cre+/− mice and their WT littermates reveal that the presence of Cre does not negatively impact retinal structure or function. There was no significant difference between cone-mediated (photopic) or rod-mediated (scotopic) electroretinogram amplitudes in mRx-Cre+/− vs. WT mice. Similarly, optical coherence tomography revealed no significant difference in retinal structure (ONL thickness) between strains. NH—natural history. ONL—outer nuclear layer

FIG. 10 shows retinal cryosections from Fxn fl/fl mice imaged 2 months post-intravitreal injection with either AAV2(trpY-F)-CBA-Cre-P2A-GFP (bottom) or AAV2(trpY-F)-CBA-GFP control vector (top). Retinas were stained with an antibody raised against FXN (white arrows), and counterstained with DAPI (white diamonds). Native GFP expression was overlaid. On the left are 40× images of the entire retina showing FXN localizes mostly to RGCs and photoreceptor (PR) inner segments (IS). DAPI localizes mostly to INL and ONL. The area inside the yellow boxes is expanded on the right to appreciate GFP alone, FXN alone, or a merge of the two. Eyes expressing Cre have clear reduction of FXN signal in both RGCs and PR IS.

FIGS. 11A-11C show fundus images of the same eye taken 8 weeks post intravitreal injection (IVI) with scAAV2-smCBA-GFP and 4 weeks post IVI with scAAV2-smCBA-Cherry. GFP (FIG. 11A) and mCherry (FIG. 11B) filtered images are shown. Retinal cryo-section from this eye reveals co-localization of AAV-mediated GFP and mCherry expression (FIG. 11C). DAPI staining is indicated with white diamonds. Abbreviations: RGC, retinal ganglion cell, INL, inner nuclear layer, ONL, outer nuclear layer.

FIG. 12 shows optical coherence tomography (OCT) line scans taken from postnatal day 14 (P14), day 30 (P30) or day 60 (P60) post-injection in flFxn+/Fxn−; Cre+/−(aka mRx-FXN KO) mice (right), or flFxn+/Fxn−; Cre−/− control mice (left). Degeneration of all retinal layers is evident in flFxn+/Fxn−; Cre+/− mice as early as P14 and progressed through P60. Abbreviations: ONL, outer nuclear layer; INL, inner nuclear layer; IPL, inner plexiform layer; RNFL, retinal nerve fiber layer; GCL, ganglion cell layer.

FIG. 13 shows quantification of the thickness of RNFL, GCL+IPL, and ONL layers via OCT. Measurements were performed on scans from flFxn+/Fxn−; Cre+/− (i.e., mRx-FXN KO) eyes (n=5) and flFxn+/Fxn−; Cre−/− controls (n=8). Degeneration in all layers was observed as early as P14 and progressed over time.

FIG. 14 shows representative scotopic (rod-mediated) and photopic (cone-mediated) ERG waveforms in mRx-FXN KO mice (asterisks) or controls at P21 (top) and P60 (bottom).

FIG. 15 shows detection of FXN in retinas of WT or mRx-FXNKO mice, the heart of WT mice, or HEK293 cells transfected with FXN plasmid using the Protein Simple Jess quantification system through use of a capillary-based Western blot tool.

FIG. 16 shows quantification of the thickness of RNFL, GCL+IPL, and ONL layers of mRx-FXN KO mice at 1 month (1m) post intravitreal injection of either AAV-FXN (left bars) or AAV-GFP control (right bars) via OCT. **-p<0.0001, *-p<0.001

FIG. 17 shows quantification of total retinal thickness in mRX-FXN KO mice at 2 months (2m) post intravitreal injection of either AAV-FXN (left bar) or AAV-GFP (right bar). **-p<0.0001

FIG. 18 shows retinal sections from mRx-FXN KO mice treated with P2-V1(Y-F+T-V)-CBA-FXN/HA-WPRE and stained with antibodies directed against FXN (left), or HA (right) and counterstained with DAPI (white diamonds). FXN staining is indicated with white arrows.

FIG. 19 shows retinal sections from mRx-FXN KO mice treated with P2-V1(Y-F+T-V)-CBA-GFP and stained with antibodies directed against FXN (left), and counterstained with DAPI (white diamonds). FXN staining is indicated with white arrows. GFP expression level is native.

FIG. 20 shows the vector map for pTR-CBA-FXN-HA-WPRE (6443 bp in length).

FIG. 21 shows the vector map for pTR-Syn-FXN-HA-WPRE (5393 bp in length).

### DETAILED DESCRIPTION

FA is a peripheral neuropathy involving a loss of sensory neurons. Studies of biopsies from patients suggest that axonal dysfunction precedes the death of neurons in a dying-back process. Therefore, gene supplementation therapy may include expression of FXN in peripheral axons to address this dysfunction and prevent axonal loss leading to neuronal death. Many of the affected neurons in FA are quite long. For example, motor neurons may be 1 meter in length, and the distance from RGC cell bodies to where the axons terminate in the brain is 50 mm. Mitochondria span the length of neurons and since frataxin is a protein that functions in the mitochondria, effective therapy may require frataxin to biodistribute to mitochondria in peripheral axons. To accomplish this an AAV vector comprising a frataxin gene and an axon targeting motif (ATM) was engineered.

With these novel vectors, the number of patients amenable to treatment may increase to greater than 30%. This would be true for advanced stage FA patients who are older/more likely to harbor pre-existing AAV2 neutralizing antibodies (NAbs), and patients that were treated previously with a systemic AAV therapy (such as the AAV9-FXN based therapy currently under consideration). Incorporation of rAAV-FXN into these novel “neutralizing antibody avoidance” capsids may preserve RGC function/structure, and vision in FA patients.

Accordingly, the present disclosure provides novel rAAV vectors, compositions, and methods, for administration of rAAV particles for treatment of FA. These vectors are designed for delivery of a therapeutic agent comprising a synthetic frataxin gene to mammalian subjects, such as human subjects. Advantageously, the novel methods of rAAV particle administration disclosed herein have improved efficiency in transducing the retina of the mammalian eye, and in particular, in transducing retinal cells in vivo, including transduction of retinal ganglion cells. Specifically, the disclosed rAAV vectors and compositions are capable of lateral spread beyond the site of vector injection. The disclosed methods may thus provide for safe and efficient delivery of the frataxin (FXN) gene to photoreceptors. The disclosure also provides cells, such as host cells, containing any of the disclosed rAAV vectors.

Delivery of any of the disclosed AAV-hFXN gene therapies may arrest or reverse disease progression after onset. In some embodiments, delivery reverses visual loss or optic nerve atrophy in the subject. In some embodiments, delivery reverses the loss of retinal ganglion cells, reverses thinning of the nerve fiber layer, nystagmus, and/or reverses loss of visual field. Accordingly, the present disclosure provides methods of administration of any of the disclosed rAAV vectors reverses visual loss or optic nerve atrophy in the subject.

In particular embodiments, delivery of this therapeutic agent a) preserves one or more RGC cells, b) restores nerve fiber layer or optic nerve head integrity, c) restores one or more rod- and/or cone-mediated functions, d) restores completely or partially visual behavior in one or both eyes, or e) any combination thereof. In some embodiments, production of the therapeutic agent persists in one or more RGCs substantially for a period of at least six months following an initial administration of the rAAV particle into the one or both eyes of the mammal.

In some embodiments, a polynucleotide comprising a heterologous nucleic acid that is encapsidated into an rAAV particle is provided. In particular embodiments, the heterologous nucleic acid is administered to the subject to provide a functional protein, e.g., human FXN, to restore, e.g., completely or partially, photoreceptor function to a subject (e.g., a human) as well as restore optic nerve head integrity, nerve fiber layer integrity, and axon structure. In some embodiments, one or both alleles of a target coding sequence of the subject are silenced by administering an rAAV particle comprising a heterologous nucleic acid disclosed herein to the subject (e.g., to a human suffering from FA).

In some aspects, the disclosure provides compositions comprising a rAAV particle and a pharmaceutically acceptable carrier, excipient, diluent and/or buffer. In some aspects, the disclosure provides a method of transducing photoreceptor cells such as RGCs to modulate expression of the heterologous nucleic acid (or transgene) in a subject, the method comprising administering to the subject, such as a human subject, a composition comprising an rAAV particle as described herein and a pharmaceutically acceptable carrier, excipient, diluent, buffer, and any combination thereof. In some aspects, the disclosure provides a method of treating FA in a subject, the method comprising administering a composition to the eye of a subject.

In some aspects, the disclosure provides a composition for use in treating retinal disease and a composition for use in the manufacture of a medicament to treat retinal disease. In some aspects, the disclosure provides a composition comprising an rAAV particle as described herein for use in treatment by subretinally or intravitreally administering to one or both eyes of the mammal.

In some aspects, administration (e.g., subretinal administration) of the disclosed rAAV vectors in doses reduced relative to standard clinical doses is provided. Some research suggests administration of high doses of AAV particles causes inflammation. Systemic administration of high doses of AAV particles that target the liver, neurons and/or muscles have led to deaths of subjects in a handful of clinical trials following excessive inflammation and onset of liver disease. The first clinical trials utilized intravitreally (IVT) delivered AAVs because the risk:benefit ratio precluded use of subretinal injection for the treatment of FA. With the advent of intra-operative optical coherence tomography (OCT), vector placement can be tightly controlled by surgeons. AAV vectors that can laterally spread beyond the injection site will lead to efficient transduction of retinal layers containing RGCs.

In some aspects, the disclosed rAAV vectors utilize capsid variants demonstrating effective tropism to the RGC tissue and/or evasion of neutralizing antibodies, such as P2-V1, P2-V1(Y-F+T-V), P2-V2, and P2-V3.

In some aspects, the disclosed rAAV vectors may utilize capsid variants that exhibit increased lateral spread and high transduction efficiencies, such as AAV44.9(E531D) and other AAV44.9 variants (e.g., AAV44.9(T492V+E531D), AAV44.9(Y446F+E531D), and AAV44.9(Y446F+T492V+E531D)). The high efficiency and lateral spread mediated by AAV44.9(E531D) allows for smaller injection bleb volumes, thereby further reducing risk by limiting the area of detachment. Accordingly, the use of any of the disclosed rAAV vectors may facilitate reduction of bleb volumes and/or doses for ocular administration necessary to achieve a therapeutic effect in subjects, such as human subjects. Methods of administration of the disclosed rAAV vectors may enable targeting of subretinal injections to areas of the retina (e.g., the retina of an FA patient) that do not contain lesions or are relatively intact. These methods may be facilitated by the use of intra-operative OCT to guide the vitreoretinal surgeon's placement of blebs.

In some embodiments, the disclosed rAAV particles are administered intravitreally. In some such cases, the rAAV particles may be administered in a total volume of less than 250 μL, less than 200 μL, less than 175 μL, less than 150 μL, less than 125 μL, or less than 100 μL. For instance, the rAAV particles may be administered in a total volume of about 100 μL, to about 150 μL. In the non-human primate experiments of the Examples provided herein, a total of 90 μL (3 inidividual injection blebs of 30 μL each) was administered subretinally to primate retina. These volumes are substantially smaller than the volumes used in standard clinical application of AAV gene therapy. For example, Luxturna (voretigene neparvovec-rzyl), an rAAV-RPE65 vector, is delivered in a 300 μL subretinal injection.

### FXN Heterologous Nucleic Acids

In some embodiments, the heterologous nucleic acid of any of the polynucleotides of the disclosure has a sequence that has at least 80% identity, at least 85% identity, at least 90% identity, at least 95% identity, at least 98%, at least 99% identity, or 100% identity to a nucleotide sequence set forth as SEQ ID NO: 18, encoding human FXN (hFXN).

In some embodiments, an ATM of a 3′ UTR is positioned downstream of the FXN coding sequence, and the resulting construct is referred to herein as “hFXN-ATM.” In some embodiments, an AAV vector herein comprises a heterologous nucleic acid comprising an FXN coding sequence, and a 3′ UTR comprising one or more ATMs. For instance, the heterologous nucleic acid is an hFXN-ATM. In some embodiments, the heterologous nucleic acid comprises an FXN coding sequence that comprises a sequence with at least 80% identity, at least 85% identity, at least 90% identity, at least 95% identity, at least 98% identity, at least 99% identity or 100% identity to SEQ ID NO: 18. In some embodiments, the 3′ UTR and/or ATM comprises at least 80% identity, at least 85% identity, at least 90% identity, at least 95% identity, at least 98% identity, at least 99% identity or 100% identity to any one of SEQ ID NOS: 9 and 20-23. In some embodiments, the ATM comprises any of the sequences set forth as SEQ ID NOs: 9 and 20-23. In particular embodiments, the 3′ UTR and/or ATM of the disclosed vectors comprises the sequence of SEQ ID NO: 9.

In some embodiments, the hFXN-ATM sequence comprises a sequence with at least 80% identity, at least 85% identity, at least 90% identity, at least 95% identity, at least 98% identity, at least 99% identity or 100% identity to any one of SEQ ID NOS: 8 and 24-27. In some embodiments, the hFXN-ATM sequence comprises the sequence of any one of SEQ ID NOS: 8 and 24-27. Non-limiting example nucleotide sequences encoding a synthetic human frataxin (FXN) transgene and comprising an ATM are shown below, where the hFXN is in lowercase and the ATM is in uppercase.

hFXN and β-Actin (e.g., ACTB) ATM (hFXN-β-Actin ATM):

(SEQ ID NO: 8)
atgtggactctcgggcgccgcgcagtagccggcctcctggcgtcaccca
gcccagcccaggcccagaccctcacccgggtcccgcggccggcagagtt
ggccccactctgcggccgacgtggcctgcgcaccgacatcgatgcgacc
tgcacgccccgccgcgcaagttcgaaccaacgtggcctcaaccagattt
ggaatgtcaaaaagcagagtgtctatttgatgaatttgaggaaatctgg
aactttgggccacccaggctctctagatgagaccacctatgaaagacta
gcagaggaaacgctggactctttagcagagttttttgaagaccttgcag
acaagccatacacgtttgaggactatgatgtctcctttgggagtggtgt
cttaactgtcaaactgggtggagatctaggaacctatgtgatcaacaag
cagacgccaaacaagcaaatctggctatcttctccatccagtggaccta
agcgttatgactggactgggaaaaactgggtgtactcccacgacggcgt
gtccctccatgagctgctggccgcagagctcactaaagccttaaaaacc
aaactggacttgtcttccttggcctattccggaaaagatgcttgaACCG
GACTGTTACCAACACCCACACCCCTGTGATGAAACAAAACCCATAAATG
C

hFXN and Synapsin (e.g., SYN1) ATM (hFXN-Synapsin ATM):

(SEQ ID NO: 24)
atgtggactctcgggcgccgcgcagtagccggcctcctggcgtcaccca
gcccagcccaggcccagaccctcacccgggtcccgcggccggcagagtt
ggccccactctgcggccgacgtggcctgcgcaccgacatcgatgcgacc
tgcacgccccgccgcgcaagttcgaaccaacgtggcctcaaccagattt
ggaatgtcaaaaagcagagtgtctatttgatgaatttgaggaaatctgg
aactttgggccacccaggctctctagatgagaccacctatgaaagacta
gcagaggaaacgctggactctttagcagagttttttgaagaccttgcag
acaagccatacacgtttgaggactatgatgtctcctttgggagtggtgt
cttaactgtcaaactgggtggagatctaggaacctatgtgatcaacaag
cagacgccaaacaagcaaatctggctatcttctccatccagtggaccta
agcgttatgactggactgggaaaaactgggtgtactcccacgacggcgt
gtccctccatgagctgctggccgcagagctcactaaagccttaaaaacc
aaactggacttgtcttccttggcctattccggaaaagatgcttgaCCAG
GACGAGGTGAAAGCTGAGACCATCCGCAGCCTGAGGAAGTCTTTCGCCA
GCCTCTTCTCCGACTGACATCCCACTCTGAGAACCCCTAAACCCCTAGA
CAGCCCTCTCTGGGTCCTGAGTCCATTTCTCACTTTTGGAACCTCCAAA
TCCCTTGAGAACCCCTCTCCTGGTTCTCCTAGAGCCCACTTCTCATTCC
TCAGTATGTCCCTTGAGAAACCTGGTCCTAAATCCAGTTCTCACATTTG
GGAATCCCCAAGTCCATTTAGAATCCTGTTCCTGGTCACCTCCAGATCT
GTTTGAGAACCTCCAAACTCCTGAAGACCTCTACTTCTGGTACATCTAA
AGGGTGATTTCTTCCCTGAAAGTTCCTGAATGGCAGAAAACCTGTTTCT
GACCTAATACATCGAATCCTCTCCTGGAGCTCTGAAATTCCTGAAAAAC
CCTATTCTACTCTTGGTCCTGAATCTCTCCAACACACCTTGCTCCTAAT
CAGAAATTTCCAATTCCATAAGAAGCCCCTATCCTTAAACCTCTTCCAT
AGGTCATCTCTCTGGAGATCCACATCTTCAAATGCCACCTACCATCCAC
CAAGCAGTCCCTCAAGGACTTCCCTTCACCTCTTCCTCTCCATCCCATT
CAATATACTGGAAGTTCCTTCCACTTCTAGGACCCCTAAGTTCCCTCCT
CCAGAAACCCCTCCCCAATTTCCTGCCTCTGACAGCTGTCTTTAAATAT
GCAAACTCCACCCATCCTCAGAATCCTTTGCACAAGGAAGGCCTGTGGT
CTCCCACTCCCCTACCTTTGCCAATGTGTTTATCTGTGACTGACTTGGC
CTCCTTTTGTGCCATGCTTGGCATATGTGGTCCTCATTCATGCCGCCTG
TGGTAATGCGTACAGTGGCACTGTTTTTGTGGTCTGCATCTTCCTCCAA
CCTCCACTCCTTGCCTGGACTTGCCCCACCCCTCAGCGGCTCTGAACCC
CCAAGAGAAGAGTCGGGAAGCAAAATAAACAAGCAAAGGCCCAGCAGAA

hFXN and β-Tubulin (e.g., Tubb2b) ATM (hFXN-β-Tubulin ATM):

(SEQ ID NO: 25)
atgtggactctcgggcgccgcgcagtagccggcctcctggcgtcaccca
gcccagcccaggcccagaccctcacccgggtcccgcggccggcagagtt
ggccccactctgcggccgacgtggcctgcgcaccgacatcgatgcgacc
tgcacgccccgccgcgcaagttcgaaccaacgtggcctcaaccagattt
ggaatgtcaaaaagcagagtgtctatttgatgaatttgaggaaatctgg
aactttgggccacccaggctctctagatgagaccacctatgaaagacta
gcagaggaaacgctggactctttagcagagttttttgaagaccttgcag
acaagccatacacgtttgaggactatgatgtctcctttgggagtggtgt
cttaactgtcaaactgggtggagatctaggaacctatgtgatcaacaag
cagacgccaaacaagcaaatctggctatcttctccatccagtggaccta
agcgttatgactggactgggaaaaactgggtgtactcccacgacggcgt
gtccctccatgagctgctggccgcagagctcactaaagccttaaaaacc
aaactggacttgtcttccttggcctattccggaaaagatgcttgaGTTC
CCCAGGCCAAGCAGGTTAGGGAAAGCTGAGATGAAAGGAGGGGGTGGGG
GGGCTTAATCTGTGAAAATACCTTGGCAGTTGAAGGAAGGAGAATGGTC
TTAGGTTTGTGCTGGGTCTCTGGTGCTCTTCACTGTTGCCTGTCACTTT
TTTTCTCTTTTTGTAATACCGATAACATCAATGTAACACTTGAGATCTT
TCTGAACTCCTGTTGTAATGGCTAAAATCACATAAACCTTTGTGTCCTA
ACGGTGTCCTCTTTTCTTTCTCTTCCTTTCTCCCTATCAAGCTCTTTGT
TATCAACTTAAATCCACCTTTCTGAACACAGAAAATTTTCTTCCTTTAG
AAAAGACTGAAAGCTCAGGTGTTTGTTTCTTCTTTGGGTATGCTATTAA
TATAAGTTGAACCAAAAATGGCCTTACTCAATCCAACAATGAGAAGAAA
CAATGGATTTTAAGATGTCCTTTGGTACACGACTTGTTTATTGAGAGTG
TGTTTTTATGAAGATGTTGCCAGACCTTTATTTCCTTAAAGGTTTATTT
GAGCAGTTACTTAGAGGCACACAGCACAGAATTTAAAGGAGAGTGAAAA
ATTAGTTTCCTCTTCTGTGTGCTTTGATCCAGCCTTCCTCCCTGAAGAA
TAGCATTATCAATAATGTTGATCATTGATAGTAGTCACCAAGTTATATC
ATTTTTATGATGCTGGGAATCAAACCCAGGGCC

hFXN and Y3 Noncoding RNA (e.g., U34826) ATM (hFXN-Y3 ncRNA ATM):

(SEQ ID NO: 26)
atgtggactctcgggcgccgcgcagtagccggcctcctggcgtcaccca
gcccagcccaggcccagaccctcacccgggtcccgcggccggcagagtt
ggccccactctgcggccgacgtggcctgcgcaccgacatcgatgcgacc
tgcacgccccgccgcgcaagttcgaaccaacgtggcctcaaccagattt
ggaatgtcaaaaagcagagtgtctatttgatgaatttgaggaaatctgg
aactttgggccacccaggctctctagatgagaccacctatgaaagacta
gcagaggaaacgctggactctttagcagagttttttgaagaccttgcag
acaagccatacacgtttgaggactatgatgtctcctttgggagtggtgt
cttaactgtcaaactgggtggagatctaggaacctatgtgatcaacaag
cagacgccaaacaagcaaatctggctatcttctccatccagtggaccta
agcgttatgactggactgggaaaaactgggtgtactcccacgacggcgt
gtccctccatgagctgctggccgcagagctcactaaagccttaaaaacc
aaactggacttgtcttccttggcctattccggaaaagatgcttgaAACA
CATATCAGATGCTGTTATATATGTGTAGTGGCTATTCCTGGTTGTCAAC
TTGACAATATTTGGAATGAACTACAATCCGGAAGCCCTGTAAGTCGTTG
AAATGTAAATAGATTTTTCTTCTAAATTAAATACTTTTCTGTGGTTTTA
TTATTACGTATGAGGTATGTATTTGGAATACACAAACATATTTTTAACA
GTTTATGCTCATAATCCACCATGACTTGTAAAGGGTGAATATTATAAAC
CTTATATATCTACAACTACAGGCCTAACTTTCGGTTGGTCCGAGAGTAG
TGGTGTTTACAACTAATTGATCACAACCAGTTACAGATTTCTTTGTTCC
TTCTCCGCTCCCACTGCTTCACTTGACCAGCCTTTTGTTGAATGAGCTA
TTAACATTCCCTCCTG

hFXN and hFXN 3′ UTR ATM (hFXN-FXN ATM):

(SEQ ID NO: 27)
atgtggactctcgggcgccgcgcagtagccggcctcctggcgtcaccca
gcccagcccaggcccagaccctcacccgggtcccgcggccggcagagtt
ggccccactctgcggccgacgtggcctgcgcaccgacatcgatgcgacc
tgcacgccccgccgcgcaagttcgaaccaacgtggcctcaaccagattt
ggaatgtcaaaaagcagagtgtctatttgatgaatttgaggaaatctgg
aactttgggccacccaggctctctagatgagaccacctatgaaagacta
gcagaggaaacgctggactctttagcagagttttttgaagaccttgcag
acaagccatacacgtttgaggactatgatgtctcctttgggagtggtgt
cttaactgtcaaactgggtggagatctaggaacctatgtgatcaacaag
cagacgccaaacaagcaaatctggctatcttctccatccagtggaccta
agcgttatgactggactgggaaaaactgggtgtactcccacgacggcgt
gtccctccatgagctgctggccgcagagctcactaaagccttaaaaacc
aaactggacttgtcttccttggcctattccggaaaagatgcttgaTCCC
CAGCCCCGTTTTAAGGACATTAAAAGCTATCAGGCCAAGACCCCAGCTT
CATTATGCAGCTGAGGTCTGTTTTTTGTTGTTGTTGTTGTTTATTTTTT
TTATTCCTGCTTTTGAGGACAGTTGGGCTATGTGTCACAGCTCTGTAGA
AAGAATGTGTTGCCTCCTACCTTGCCCCCAAGTTCTGATTTTTAATTTC
TATGGAAGATTTTTTGGATTGTCGGATTTCCTCCCTCACATGATACCCC
TTATCTTTTATAATGTCTTATGCCTATACCTGAATATAACAACCTTTAA
AAAAGCAAAATAATAAGAAGGAAAAATTCCAGGAGGGAAAATGAATTGT
CTTCACTCTTCATTCTTTGAAGGATTTACTGCAAGAAGTACATGAAGAG
CAGCTGGTCAACCTGCTCACTGTTCTATCTCCAAATGAGACACATTAAA
GGGTAGCCTACAAATGTTTTCAGGCTTCTTTCAAAGTGTAAGCACTTCT
GAGCTCTTTAGCATTGAAGTGTCGAAAGCAACTCACACGGGAAGATCAT
TTCTTATTTGTGCTCTGTGACTGCCAAGGTGTGGCCTGCACTGGGTTGT
CCAGGGAGACCTAGTGCTGTTTCTCCCACATATTCACATACGTGTCTGT
GTGTATATATATTTTTTCAATTTAAAGGTTAGTATGGAATCAGCTGCTA
CAAGAATGCAAAAAATCTTCCAAAGACAAGAAAAGAGGAAAAAAAGCCG
TTTTCATGAGCTGAGTGATGTAGCGTAACAAACAAAATCATGGAGCTGA
GGAGGTGCCTTGTAAACATGAAGGGGCAGATAAAGGAAGGAGATACTCA
TGTTGATAAAGAGAGCCCTGGTCCTAGACATAGTTCAGCCACAAAGTAG
TTGTCCCTTTGTGGACAAGTTTCCCAAATTCCCTGGACCTCTGCTTCCC
CATCTGTTAAATGAGAGAATAGAGTATGGTTGATTCCCAGCATTCAGTG
GTCCTGTCAAGCAACCTAACAGGCTAGTTCTAATTCCCTATTGGGTAGA
TGAGGGGATGACAAAGAACAGTTTTTAAGCTATATAGGAAACATTGTTA
TTGGTGTTGCCCTATCGTGATTTCAGTTGAATTCATGTGAAAATAATAG
CCATCCTTGGCCTGGCGCGGTGGCTCACACCTGTAATCCCAGCACTTTT
GGAGGCCAAGGTGGGTGGATCACCTGAGGTCAGGAGTTCAAGACCAGCC
TGGCCAACATGATGAAACCCCGTCTCTACTAAAAATACAAAAAATTAGC
CGGGCATGATGGCAGGTGCCTGTAATCCCAGCTACTTGGGAGGCTGAAG
CGGAAGAATCGCTTGAACCCAGAGGTGGAGGTTGCAGTGAGCCGAGATC
GTGCCATTGCACTGTAACCTGGGTGACTGAGCAAAACTCTGTCTCAAAA
TAATAATAACAATATAATAATAATAATAGCCATCCTTTATTGTACCCTT
ACTGGGTTAATCGTATTATACCACATTACCTCATTTTAATTTTTACTGA
CCTGCACTTTATACAAAGCAACAAGCCTCCAGGACATTAAAATTCATGC
AAAGTTATGCTCATGTTATATTATTTTCTTACTTAAAGAAGGATTTATT
AGTGGCTGGGCATGGTGGCGTGCACCTGTAATCCCAGGTACTCAGGAGG
CTGAGACGGGAGAATTGCTTGACCCCAGGCGGAGGAGGTTACAGTGAGT
CGAGATCGTACCTGAGCGACAGAGCGAGACTCCGTCTCAAAAAAAAAAA
AAAGGAGGGTTTATTAATGAGAAGTTTG

By a nucleic acid molecule (e.g., a heterologous nucleic acid) comprising a nucleotide sequence having at least, for example, 95% “identity” to a query nucleic acid sequence, it is intended that the nucleotide sequence of the subject nucleic acid molecule is identical to the query sequence except that the subject nucleic acid molecule sequence may include up to five nucleotide alterations per each 100 nucleotides of the query sequence. In other words, to obtain a promoter having a nucleotide sequence at least 95% identical to a reference (query) sequence, up to 5% of the nucleotides in the subject sequence may be inserted, deleted, or substituted with another nucleotide. These alterations of the reference sequence may occur at the 5′ or 3′ ends of the reference sequence or anywhere between those positions, interspersed either individually among nucleotides in the reference sequence or in one or more contiguous groups within the reference sequence.

As a practical matter, whether any particular nucleic acid molecule is at least 80%, 85%, 90%, 95%, 96%, 97%, 98%, or 99% identical to, for instance, the nucleotide sequence of a synthetic FXN cDNA, can be determined conventionally using known computer programs. Preferred methods for determining the best overall match between a query sequence (a sequence of the present disclosure) and a subject sequence, also referred to as a global sequence alignment, can be determined using the FASTA program analysis described by Pearson and Lipman (1988) and FASTDB and blastn computer programs based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the 5′ and/or 3′ ends, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

In some embodiments, the polynucleotides of the rAAV vectors described herein may comprise a heterologous nucleotide acid comprising a sequence having 1, 2, 3, 4, 5, 6, 7, 8, 9, 10, 11, 12, 13, 14, 15, 16, 17, or 18 nucleotides that differ relative to the sequence as set forth in any one of SEQ ID NOs: 8, 9, 18, and 20-27. These differences may comprise nucleotides that have been inserted, deleted, or substituted relative to the sequence of any one of SEQ ID NOs: 8, 9, 18, and 20-27. In some embodiments, the polynucleotides comprise truncations at the 5′ or 3′ end relative to any one of SEQ ID NOs: 8, 9, 18, and 20-27. In some embodiments, the disclosed polynucleotides contain stretches of about 50, about 75, about 100, about 125, about 150, about 175, or about 180 nucleotides in common with the sequence of any one of SEQ ID NOs: 8, 9, 18 and 20-27. In some embodiments, the disclosed polynucleotides contain stretches of about 50, about 75, about 100, about 125, about 150, about 175, about 180, about 200, about 300, about 400, about 500, about 600, about 700, about 800, about 900, about 1000, or more than about 1000 nucleotides in common with the sequence of any one of SEQ ID NOs: 8, 9, 18 and 20-27. The polynucleotides of the disclosure may comprise 1, 2, 3, 4, 5, 6, 7, 8, 9, 10, 11, 12 or more than 12 silent mutations that do not result in mutations in the encoded FXN protein sequence. In some embodiments, the disclosed polynucleotides may comprise between 30 and 40 silent mutations relative to the wild-type FXN sequence. In some embodiments, the disclosed polynucleotides may comprise between 2 and 10 silent mutations. In some embodiments, the polynucleotides of the disclosure may comprise 4 silent mutations. In some embodiments, the silent mutation(s) is a CGC to CGA mutation. In some embodiments, the polynucleotide of the rAAV vector comprises an HA tag. In some embodiments, the polynucleotide of the rAAV vector does not comprise an HA tag.

In some embodiments, the heterologous nucleic acid of any of the rAAV nucleic acid vectors of the disclosure has a sequence that has at least 80% identity, at least 75% identity, at least 90% identity, at least 95% identity, at least 98%, at least 99% identity, or 100% identity to the nucleotide sequence set forth as SEQ ID NO: 18. The heterologous nucleic acid may comprise SEQ ID NO: 18.

In various embodiments, the rAAV vectors comprising a heterologous nucleic acid encode a variant of the human frataxin 1 (FXN) protein. In some embodiments, the amino acid sequence of the FXN protein is shown below as SEQ ID NO: 12 or 14. In some embodiments, the encoded frataxin protein is a human frataxin protein defined by the amino acid sequence of SEQ ID NO: 12. In some embodiments, the encoded FXN protein may comprise a sequence having 1, 2, 3, 4, 5, 6, 7, 8, 9, 10, 11, 12, 13, 14, 15, 16, 17, or 18 amino acids that differ relative to the sequence of any one of SEQ ID NOs: 12 and 14. In some embodiments, the encoded FXN protein may comprise a sequence having 1, 2, 3, 4, 5, 6, 7, 8, 9, 10, 11, 12, 13, 14, 15, 16, 17, or 18 amino acids that differ relative to SEQ ID NO: 12. These differences may comprise amino acids that have been inserted, deleted, or substituted relative to the sequence of any one of SEQ ID NOs: 12 and 14. In some embodiments, the disclosed rAAV vectors encode a protein having an amino acid sequence having at least 75% identity, at least 80% identity, at least 85% identity, at least 90% identity, at least 92.5% identity, at least 95% identity, at least 98%, at least 99% identity, or 100% identity to any of the amino acid sequences of SEQ ID NOs: 12 and 14. In some embodiments, the FXN protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of SEQ ID NO: 12.

### Exemplary Human FXN Protein

(SEQ ID NO: 12)
MWTLGRRAVAGLLASPSPAQAQTLTRVPRPAELAPLCGRRGLRTDIDAT
CTPRRASSNQRGLNQIWNVKKQSVYLMNLRKSGTLGHPGSLDETTYERL
AEETLDSLAEFFEDLADKPYTFEDYDVSFGSGVLTVKLGGDLGTYVINK
QTPNKQIWLSSPSSGPKRYDWTGKNWVYSHDGVSLHELLAAELTKALKT
KLDLSSLAYSGKDA

### HA-Tagged Human FXN Protein; HA Tag Underlined

(SEQ ID NO: 14)
MWTLGRRAVAGLLASPSPAQAQTLTRVPRPAELAPLCGRRGLRTDIDAT
CTPRRASSNQRGLNQIWNVKKQSVYLMNLRKSGTLGHPGSLDETTYERL
AEETLDSLAEFFEDLADKPYTFEDYDVSFGSGVLTVKLGGDLGTYVINK
QTPNKQIWLSSPSSGPKRYDWTGKNWVYSHDGVSLHELLAAELTKALKT
KLDLSSLAYSGKDAYPYDVPDYA

### Promoters

In some embodiments, the polynucleotide within the rAAV particle comprises regulatory sequences, such as transcription and translation initiation and termination codons, which are specific to the type of host (e.g., bacterium, fungus, plant, or animal) into which the rAAV particle is to be introduced. Preferably, the nucleic acid molecule within the rAAV particle comprises regulatory sequences that are specific to the genus of the host. Most preferably, the molecule comprises regulatory sequences that are specific to the species of the host. The polynucleotide within the rAAV particle may comprise expression control sequences, such as promoters, enhancers, polyadenylation signals, transcription terminators, internal ribosome entry sites (IRES), and the like, that provide for the expression of the heterologous nucleic acid(s) in a host cell. Exemplary expression control sequences are known in the art. In some embodiments, the heterologous nucleic acid is operably linked to one or more regulatory sequences which direct expression of the heterologous nucleic acid in a retinal ganglion cell.

In some embodiments, the polynucleotide of any of the disclosed rAAV vectors contains an endogenous promoter, i.e. a frataxin promoter such as a human frataxin promoter (hFXNPro). In some embodiments, the polynucleotide of any of the disclosed rAAV vectors contain an exogenous promoter, such as synapsin.

In some embodiments, the polynucleotide of any of the disclosed rAAV vectors comprises a promoter that is capable of expressing the nucleic acid sequence in one or more photoreceptors or RGCs of a mammalian eye. In particular embodiments, the disclosure provides a retinal tissue-specific promoter operably linked to at least a first hetereologous nucleic acid sequence that encodes a therapeutic agent. Exemplary PR-cell-specific, RGC-specific, and/or RPE-specific promoters may comprise a) photoreceptor-specific promoters (active in rod and cone cells), e.g., IRBP promoter (hIRPB, IRBP, IRBP241), rhodopsin kinase promoter (hGRK1, GRK1, GRK, RK), and/or chimeric human Frataxin-IRBP enhancer (RS/IRPB); cone-specific promoters, e.g., red/green cone opsin promoter (which may comprise the 2.1 kb (PR2.1) version or 1.7 kb (PR1.7) version, see U.S. Patent Publication No. 2018/0112231), Cone Arrestin promoter (hCAR, CAR), chimeric IRBP enhancer-cone transducin promoter (IRBP/GNAT2, IRBPe-GNAT2); rod-specific promoters, e.g., human rhodopsin promoter (RHO, RHOP, etc.), human NRL promoter (NRL); or RPE-specific promoters such as RPE65 or Bestrophin/VMD2 (BEST1, BEST, VMD2). In some embodiments, the promoter is a photoreceptor-specific promoter such as an IRBP promoter (hIRPB, IRBP, IRBP241). In some embodiments, the promoter is a rod-specific promoter such as a human rhodopsin promoter (RHO, RHOP).

Non-limiting example RGC promoters include synapsin (SYN1), Nefh(Ple345) (e.g., human and mouse), Sncg (e.g., human and mouse), Cx36 (e.g., human), and dcx (e.g., human).

In some embodiments, the polynucleotide comprises a synapsin (Syn1) promoter, such as a human synapsin 1 (hSyn1) promoter. In exemplary embodiments, the polynucleotide comprises an EF1-alpha (EF1-α) promoter. In some embodiments, the polynucleotide comprises a chicken beta actin promoter (CBA) promoter. In some embodiments, the polynucleotide comprises a truncated chimeric CBA-CMV promoter (smCBA) promoter (sometimes referred to as a CAG promoter), which contains a CMV enhancer and a truncated CBA promoter. In some embodiments, the polynucleotide comprises a desmin (Des) promoter. In some embodiments, the polynucleotide comprises a 3-phosphoglycerate kinase (PGK-1) promoter (see Adra C N. et al. 1987. Gene 60(1):65-74).

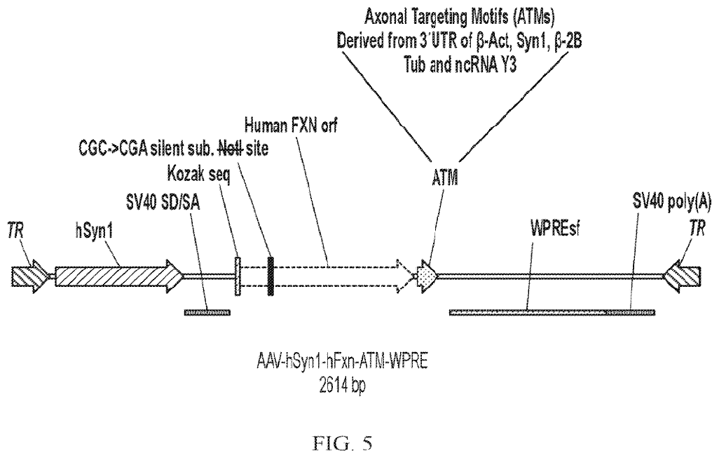

Exemplary vectors of the disclosure that comprise hSyn1 promoters include AAV-hSyn1-hFXN-ATM and AAV-hSyn1-hFXN-ATM-WPREsf (FIG. 5). An exemplary vector of the disclosure that comprise an smCBA promoter is AAV-CBA-hFXN-ATM.

In some embodiments, the promoter of any of the disclosed rAAV vectors comprises a nucleotide sequence that is at least 95%, at least 98%, at least 99%, or 100% identical the sequence of the hSyn1 promoter as set forth in SEQ ID NO: 31:

(SEQ ID NO: 31)
GATCTAGGCCTACTAGTCTGCAGAGGGCCCTGCGTATGAGTGCAAGTGG
GTTTTAGGACCAGGATGAGGCGGGGTGGGGGTGCCTACCTGACGACCGA
CCCCGACCCACTGGACAAGCACCCAACCCCCATTCCCCAAATTGCGCAT
CCCCTATCAGAGAGGGGGAGGGGAAACAGGATGCGGCGAGGCGCGTGCG
CACTGCCAGCTTCAGCACCGCGGACAGTGCCTTCGCCCCCGCCTGGCGG
CGCGCGCCACCGCCGCCTCAGCACTGAAGGCGCGCTGACGTCACTCGCC
GGTCCCCCGCAAACTCCCCTTCCCGGCCACCTTGGTCGCGTCCGCGCCG
CCGCCGGCCCAGCCGGACCGCACCACGCGAGGCGCGAGATAGGGGGGCA
CGGGCGCGACCATCTGCGCTGCGGCGCCGGCGACTCAGCGCTGCCTCAG
TCTGCGGTGGGCAGCGGAGGAGTCGTGTCGTGCCTGAGAGCGCAGTC

In some embodiments, the promoter of any of the disclosed rAAV vectors comprises a nucleotide sequence that is at least 95%, at least 98%, at least 99%, or 100% identical to the sequence of the smCBA promoter as set forth in SEQ ID NO: 28:

(SEQ ID NO: 28)
AATTCGGTACCCTAGTTATTAATAGTAATCAATTACGGGGTCATTAGTT
CATAGCCCATATATGGAGTTCCGCGTTACATAACTTACGGTAAATGGCC
CGCCTGGCTGACCGCCCAACGACCCCCGCCCATTGACGTCAATAATGAC
GTATGTTCCCATAGTAACGCCAATAGGGACTTTCCATTGACGTCAATGG
GTGGACTATTTACGGTAAACTGCCCACTTGGCAGTACATCAAGTGTATC
ATATGCCAAGTACGCCCCCTATTGACGTCAATGACGGTAAATGGCCCGC
CTGGCATTATGCCCAGTACATGACCTTATGGGACTTTCCTACTTGGCAG
TACATCTACGTATTAGTCATCGCTATTACCATGGTCGAGGTGAGCCCCA
CGTTCTGCTTCACTCTCCCCATCTCCCCCCCCTCCCCACCCCCAATTTT
GTATTTATTTATTTTTTAATTATTTTGTGCAGCGATGGGGGCGGGGGGG
GGGGGGGGGCGCGCGCCAGGCGGGGCGGGGCGGGGCGAGGGGCGGGGCG
GGGCGAGGCGGAGAGGTGCGGCGGCAGCCAATCAGAGCGGCGCGCTCCG
AAAGTTTCCTTTTATGGCGAGGCGGCGGCGGCGGCGGCCCTATAAAAAG
CGAAGCGCGCGGCGGGCGGGAGTCGCTGCGCGCTGCCTTCGCCCCGTGC
CCCGCTCCGCCGCCGCCTCGCGCCGCCCGCCCCGGCTCTGACTGACCGC
GTTACTCCCACAGGTGAGCGGGCGGGACGGCCCTTCTCCTCCGGGCTGT
AATTAGCGCTTGGTTTAATGACGGCTTGTTTCTTTTCTGTGGCTGCGTG
AAAGCCTTGAGGGGCTCCGGGAGCTAGAGCCTCTGCTAACCATGTTCAT
GCCTTCTTCTTTTTCCTACAGCTCCTGGGCAACGTGCTGGTTATTGTGC
TGTCTCATCATTTTGGCAAAG.

In some embodiments, the promoter of any of the disclosed rAAV vectors comprises a nucleotide sequence that is at least 95%, at least 98%, at least 99%, or 100% identical to the sequence of the CBA promoter as set forth in SEQ ID NO: 3:

(SEQ ID NO: 3)
CATGGTCGAGGTGAGCCCCACGTTCTGCTTCACTCTCCCCATCTCCCCC
CCCTCCCCACCCCCAATTTTGTATTTATTTATTTTTTAATTATTTTGTG
CAGCGATGGGGGCGGGGGGGGGGGGGGGGCGCGCGCCAGGCGGGGCGGG
GCGGGGCGAGGGGGGGGGCGGGGCGAGGCGGAGAGGTGCGGCGGCAGCC
AATCAGAGCGGCGCGCTCCGAAAGTTTCCTTTTATGGCGAGGCGGCGGC
GGCGGCGGCCCTATAAAAAGCGAAGCGCGCGGCGGGCG

The rAAV vectors of the disclosure may comprise a promoter having the sequence of SEQ ID NO: 3, SEQ ID NO: 28, or SEQ ID NO: 31. In some embodiments, the disclosure provides vectors containing constitutive promoters operably linked to at least a first polynucleotide, any of which may comprise a CMV, CBA, CB, smCBA, or CBA hybrid (CBh) promoter.

### Other Regulatory Elements

In some embodiments, the nucleic acid vector comprises a post-transcriptional regulatory sequence or a polyadenylation signal. In some embodiments, the nucleic acid vector comprises a woodchuck hepatitis virus post-transcription regulatory element (WPRE), a polyadenylation signal sequence, an intron/exon junctions/splicing signal, or any combination thereof. The polynucleotide may comprises a WPRE element, such as a WPRE element that comprises the nucleotide sequence of SEQ ID NO: 15. In some embodiments, the WPRE element is positioned 3′ of the heterologous nucleic acid. In other embodiments, the WPRE element is positioned 5′ of the heterologous nucleic acid.

In some embodiments, the WPRE is a WPREsf sequence, where the “sf” suffix denotes safe for administration. In some embodiments, the polynucleotide of any of the disclosed rAAV vectors comprises a WPRE element having at least 95%, 98%, or 99% identity to the nucleotide sequence of SEQ ID NO: 15. In some embodiments, the polynucleotide comprises the sequence of SEQ ID NO: 15.

WPREsf sequence:

(SEQ ID NO: 15)
TCGACCTCTGGATTACAAAATTTGTGAAAGATTGACTGGTATTCTTAAC
TATGTTGCTCCTTTTACGCTATGTGGATACGCTGCTTTAATGCCTTTGT
ATCATGCTATTGCTTCCCGTATGGCTTTCATTTTCTCCTCCTTGTATAA
ATCCTGGTTGCTGTCTCTTTATGAGGAGTTGTGGCCCGTTGTCAGGCAA
CGTGGCGTGGTGTGCACTGTGTTTGCTGACGCAACCCCCACTGGTTGGG
GCATTGCCACCACCTGTCAGCTCCTTTCCGGGACTTTCGCTTTCCCCCT
CCCTATTGCCACGGCGGAACTCATCGCCGCCTGCCTTGCCCGCTGCTGG
ACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCT
GCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTC
GCCTTCGCCCTCAGACGAGTCGGATCTCCCTTTGGGCCGCCTCCCCGC

Accordingly, exemplary rAAV vectors described in the disclosure may comprise any one of the following structures: AAV-CBA-hFXN-WPREsf, AAV-hSyn1-hFXN, AAV-hSyn1-hFXN-WPREsf, and AAV-hSyn1-GFP. Exemplary vectors encoding an FXN protein may comprise any of the following: AAV44.9(E531D)-hSyn1-hFXN, AAV44.9(E531D)-hSyn1-hFXN-WPREsf, P2-V1-hSyn1-hFXN, P2-V1-hSyn1-hFXN-WPREsf, P2-V1(Y-F+T-V)-hSyn1-hFXN, P2-V1(Y-F+T-V)-hSyn1-hFXN-WPREsf, AAV5-hSyn1-hFXN, AAV5-hSyn1-hFXN-WPREsf, AAV2(4pMut)ΔHS-hSyn1-hFXN, AAV2(4pMut)ΔHS-hSyn1-hFXN-WPREsf, AAV2(4pMut)ΔHS-CBA-hFXN, AAV2(4pMut)ΔHS-CBA-hFXN-WPREsf, P2-V1-CBA-hFXN, P2-V1-CBA-hFXN-WPREsf, P2-V1(Y-F+T-V)-CBA-hFXN, P2-V1(Y-F+T-V)-CBA-hFXN-WPREsf, P2-V2-CBA-hFXN, P2-V2-CBA-hFXN-WPREsf, P2-V3-CBA-hFXN, P2-V3-CBA-hFXN-WPREsf, DGE-DF-CBA-hFXN, and DGE-DF-CBA-hFXN-WPREsf, AAV8(Y447F+Y733F+T494V)-hSyn1-hFXN, or AAV8(Y447F+Y733F+T494V)-hSyn1-hFXN-WPREsf. In any of the above-described vectors, the “hFXN” component includes one or more axon-targeting motifs (ATMs) in the vector or the 3′ UTR of the hFXN trangene. Any of the above-described vectors may further contain a hemagluttinnin (HA) tag.

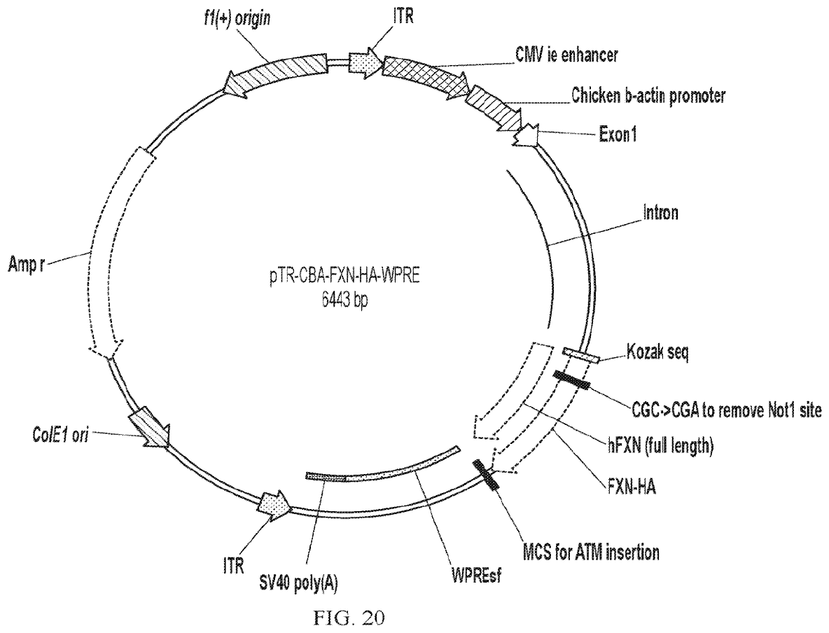

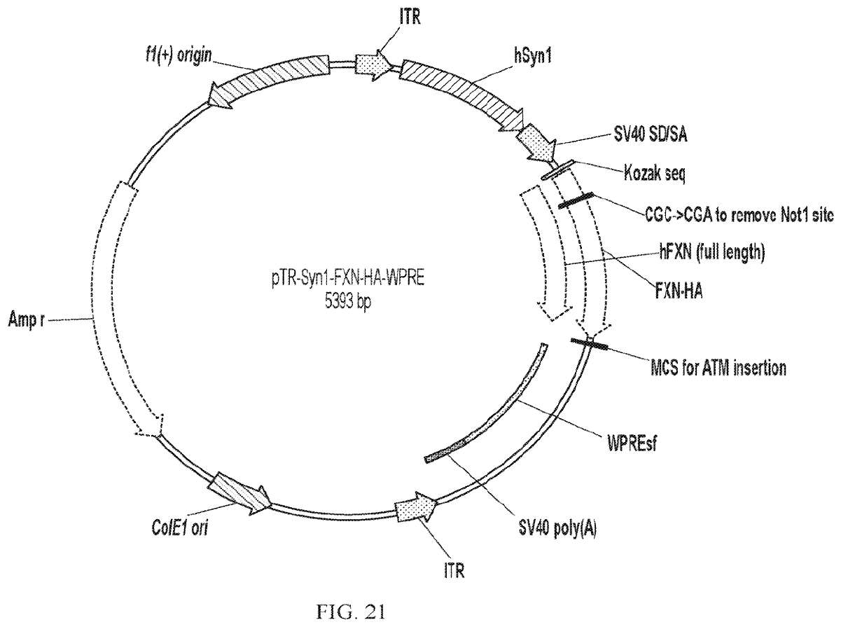

The disclosed rAAV vectors may comprise a nucleotide sequence having at least 75% identity, at least 80% identity, at least 85% identity, at least 90% identity, at least 95% identity, 98% identity, or 99% identity to the nucleotide sequence of SEQ ID NO: 16 or 17. In some embodiments, the vector comprises the nucleotide sequence of SEQ ID NO: 16 or 17. These vectors contain AAV2 ITRs flanking the polynycleotide comprising the hFXN-ATM-WPREsf sequence. These vectors further contain hemagluttinin (HA) tags. In particular embodiments, the disclosure provides rAAV vectors comprising the AAV2-CBA-hFXN-ATM-HA-WPREsf structure that comprises the nucleotide sequence of SEQ ID NO: 16, provided below. The length of SEQ ID NO: 16 is 3579 nucleotides (nt). In particular embodiments, the disclosure provides rAAV vectors comprising the AAV-hSyn1-hFXN-ATM-WPREsf structure that comprises the nucleotide sequence of SEQ ID NO: 17, provided below. The length of SEQ ID NO: 17 is 2529 nucleotides (nt). Maps for the plasmids containing the vectors of SEQ ID NOs: 16 and 17 are illustrated in FIGS. 20 and 21, respectively.

### AAV2-CBA-hFXN-ATM-HA-WPREsf

(SEQ ID NO: 16)
GGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGAGGGAGTGG
CCAACTCCATCACTAGGGGTTCCTCAGATCTGAATTCGGTACCTAGTTATTAATAGTAAT
CAATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATTTAC
GGTAAACTGCCCACTTGGCAGTACATCAAGTGTATCATATGCCAAGTACGCCCCCTATTG
ACGTCAATGACGGTAAATGGCCCGCCTGGCATTATGCCCAGTACATGACCTTATGGGACT
TTCCTACTTGGCAGTACATCTACGTATTAGTCATCGCTATTACCATGGTCGAGGTGAGCC
CCACGTTCTGCTTCACTCTCCCCATCTCCCCCCCCTCCCCACCCCCAATTTTGTATTTATTT
ATTTTTTAATTATTTTGTGCAGCGATGGGGGCGGGGGGGGGGGGGGGGCGCGCGCCAGG
CGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGC
CCTATAAAAAGCGAAGCGCGCGGCGGGCGGGAGTCGCTGCGCGCTGCCTTCGCCCCGTG
CCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCGCTTGGTTTAATGA
CGGCTTGTTTCTTTTCTGTGGCTGCGTGAAAGCCTTGAGGGGCTCCGGGAGGGCCCTTTG
TGCGGGGGGAGCGGCTCGGGGGGTGCGTGCGTGTGTGTGTGCGTGGGGAGCGCCGCGTG
CGGCTCCGCGCTGCCCGGCGGCTGTGAGCGCTGCGGGCGCGGCGCGGGGCTTTGTGCGC
TCCGCAGTGTGCGCGAGGGGAGCGCGGCCGGGGGCGGTGCCCCGCGGTGCGGGGGGGG
CTGCGAGGGGAACAAAGGCTGCGTGCGGGGTGTGTGCGTGGGGGGGTGAGCAGGGGGT
GTGGGCGCGTCGGTCGGGCTGCAACCCCCCCTGCACCCCCCTCCCCGAGTTGCTGAGCAC
GGCCCGGCTTCGGGTGCGGGGCTCCGTACGGGGCGTGGCGCGGGGCTCGCCGTGCCGGG
CGGGGGGTGGCGGCAGGTGGGGGTGCCGGGCGGGGGGGGGCCGCCTCGGGCCGGGGAG
GGCTCGGGGGAGGGGCGCGGCGGCCCCCGGAGCGCCGGCGGCTGTCGAGGCGCGGCGA
GCCGCAGCCATTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGCGGGCGCGGG
GCGAAGCGGTGCGGCGCCGGCAGGAAGGAAATGGGCGGGGAGGGCCTTCGTGCGTCGC
CGCGCCGCCGTCCCCTTCTCCCTCTCCAGCCTCGGGGCTGTCCGCGGGGGGACGGCTGCC
TTCGGGGGGGACGGGGCAGGGCGGGGTTCGGCTTCTGGCGTGTGACCGGCGGCTCTAGA
GCCTCTGCTAACCATGTTCATGCCTTCTTCTTTTTCCTACAGCTCCTGGGCAACGTGCTGG
TTATTGTGCTGTCTCATCATTTTGGCAAAGAATTCCTCGAAGATCTAGGCAACGCGTCTC
GAGGCGGCCGCCACCATGTGGACTCTCGGGCGCCGCGCAGTAGCCGGCCTCCTGGCGTC
ACCCAGCCCAGCCCAGGCCCAGACCCTCACCCGGGTCCCGCGGCCGGCAGAGTTGGCCC
CACTCTGCGGCCGACGTGGCCTGCGCACCGACATCGATGCGACCTGCACGCCCCGCCGC
GCAAGTTCGAACCAACGTGGCCTCAACCAGATTTGGAATGTCAAAAAGCAGAGTGTCTA
TTTGATGAATTTGAGGAAATCTGGAACTTTGGGCCACCCAGGCTCTCTAGATGAGACCAC
CTATGAAAGACTAGCAGAGGAAACGCTGGACTCTTTAGCAGAGTTTTTTGAAGACCTTGC
AGACAAGCCATACACGTTTGAGGACTATGATGTCTCCTTTGGGAGTGGTGTCTTAACTGT
CAAACTGGGTGGAGATCTAGGAACCTATGTGATCAACAAGCAGACGCCAAACAAGCAAA
TCTGGCTATCTTCTCCATCCAGTGGACCTAAGCGTTATGACTGGACTGGGAAAAACTGGG
TGTACTCCCACGACGGCGTGTCCCTCCATGAGCTGCTGGCCGCAGAGCTCACTAAAGCCT
TAAAAACCAAACTGGACTTGTCTTCCTTGGCCTATTCCGGAAAAGATGCTTACCCTTACG
ATGTACCGGATTATGCATGAGTCGACGCGGCCGCGCGGATCCAAAAAGCTTGGGGCTAG
CAAAATGCATGGATCCTCAACCTCTGGATTACAAAATTTGTGAAAGATTGACTGGTATTC
TTAACTATGTTGCTCCTTTTACGCTATGTGGATACGCTGCTTTAATGCCTTTGTATCATGC
TATTGCTTCCCGTATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTGTTTGCTGACG
CAACCCCCACTGGTTGGGGCATTGCCACCACCTGTCAGCTCCTTTCCGGGACTTTCGCTTT
CCCCCTCCCTATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCATCGTCCTTTCC
TTGGCTGCTCGCCTGTGTTGCCACCTGGATTCTGCGCGGGACGTCCTTCTGCTACGTCCCT
TCGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCGGA
TCCAGACATGATAAGATACATTGATGAGTTTGGACAAACCACAACTAGAATGCAGTGAA
AAAAATGCTTTATTTGTGAAATTTGTGATGCTATTGCTTTATTTGTAACCATTATAAGCTG
CAATAAACAAGTTAACAACAACAATTGCATTCATTTTATGTTTCAGGTTCAGGGGGAGGT
GTGGGAGGTTTTTTAGTCGACTGGGGAGAGATCTGAGGAACCCCTAGTGATGGAGTTGG
CCACTCCCTCTCTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGAGGGAGTGGCC

### AAV2-hSyn1-hFXN-ATM-HA-WPREsf

(SEQ ID NO: 17)
GGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGAGGGAGTGG
CCAACTCCATCACTAGGGGTTCCTCAGATCTGAATTCGGTACCGATCTAGGCCTACTAGT
CTGCAGAGGGCCCTGCGTATGAGTGCAAGTGGGTTTTAGGACCAGGATGAGGCGGGGTG
GGGGTGCCTACCTGACGACCGACCCCGACCCACTGGACAAGCACCCAACCCCCATTCCC
CAAATTGCGCATCCCCTATCAGAGAGGGGGAGGGGAAACAGGATGCGGCGAGGCGCGT
GCGCACTGCCAGCTTCAGCACCGCGGACAGTGCCTTCGCCCCCGCCTGGCGGCGCGCGCC
ACCGCCGCCTCAGCACTGAAGGCGCGCTGACGTCACTCGCCGGTCCCCCGCAAACTCCCC
TTCCCGGCCACCTTGGTCGCGTCCGCGCCGCCGCCGGCCCAGCCGGACCGCACCACGCGA
GGCGCGAGATAGGGGGGCACGGGCGCGACCATCTGCGCTGCGGCGCCGGCGACTCAGCG
CTGCCTCAGTCTGCGGTGGGCAGCGGAGGAGTCGTGTCGTGCCTGAGAGCGCAGTCGAG
AATTCACTCTAGAGGATCCGGTACTCGAGGAACTGAAAAACCAGAAAGTTAACTGGTAA
GTTTAGTCTTTTTGTCTTTTATTTCAGGTCCCGGATCCGGTGGTGGTGCAAATCAAAGAAC
TGCTCCTCAGTGGATGTTGCCTTTACTTCTAGGCCTGTACGGAAGTGTTACTTCTGCTCTA
AAAGCTGCGGAATTGTACCCGCGGCCGCCACCATGTGGACTCTCGGGCGCCGCGCAGTA
GCCGGCCTCCTGGCGTCACCCAGCCCAGCCCAGGCCCAGACCCTCACCCGGGTCCCGCG
GCCGGCAGAGTTGGCCCCACTCTGCGGCCGACGTGGCCTGCGCACCGACATCGATGCGA
CCTGCACGCCCCGCCGCGCAAGTTCGAACCAACGTGGCCTCAACCAGATTTGGAATGTCA
AAAAGCAGAGTGTCTATTTGATGAATTTGAGGAAATCTGGAACTTTGGGCCACCCAGGCT
CTCTAGATGAGACCACCTATGAAAGACTAGCAGAGGAAACGCTGGACTCTTTAGCAGAG
TTTTTTGAAGACCTTGCAGACAAGCCATACACGTTTGAGGACTATGATGTCTCCTTTGGG
AGTGGTGTCTTAACTGTCAAACTGGGTGGAGATCTAGGAACCTATGTGATCAACAAGCA
GACGCCAAACAAGCAAATCTGGCTATCTTCTCCATCCAGTGGACCTAAGCGTTATGACTG
GACTGGGAAAAACTGGGTGTACTCCCACGACGGCGTGTCCCTCCATGAGCTGCTGGCCG
CAGAGCTCACTAAAGCCTTAAAAACCAAACTGGACTTGTCTTCCTTGGCCTATTCCGGAA
AAGATGCTTACCCTTACGATGTACCGGATTATGCATGAGTCGACGCGGCCGCGCGGATCC
AAAAAGCTTGGGGCTAGCAAAATGCATGGATCCTCAACCTCTGGATTACAAAATTTGTG
AAAGATTGACTGGTATTCTTAACTATGTTGCTCCTTTTACGCTATGTGGATACGCTGCTTT
AATGCCTTTGTATCATGCTATTGCTTCCCGTATGGCTTTCATTTTCTCCTCCTTGTATAAAT
CCTGGTTGCTGTCTCTTTATGAGGAGTTGTGGCCCGTTGTCAGGCAACGTGGCGTGGTGT
GCACTGTGTTTGCTGACGCAACCCCCACTGGTTGGGGCATTGCCACCACCTGTCAGCTCC
TTTCCGGGACTTTCGCTTTCCCCCTCCCTATTGCCACGGCGGAACTCATCGCCGCCTGCCT
TGCCCGCTGCTGGACAGGGGCTCGGCTGTTGGGCACTGACAATTCCGTGGTGTTGTCGGG
GAAATCATCGTCCTTTCCTTGGCTGCTCGCCTGTGTTGCCACCTGGATTCTGCGCGGGACG
TCCTTCTGCTACGTCCCTTCGGCCCTCAATCCAGCGGACCTTCCTTCCCGCGGCCTGCTGC
CGGCTCTGCGGCCTCTTCCGCGTCTTCGCCTTCGCCCTCAGACGAGTCGGATCTCCCTTTG
GGCCGCCTCCCCGCGGATCCAGACATGATAAGATACATTGATGAGTTTGGACAAACCAC
AACTAGAATGCAGTGAAAAAAATGCTTTATTTGTGAAATTTGTGATGCTATTGCTTTATTT
GTAACCATTATAAGCTGCAATAAACAAGTTAACAACAACAATTGCATTCATTTTATGTTT
CAGGTTCAGGGGGAGGTGTGGGAGGTTTTTTAGTCGACTGGGGAGAGATCTGAGGAACC
CCTAGTGATGGAGTTGGCCACTCCCTCTCTGCGCGCTCGCTCGCTCACTGAGGCCGCCCG
GGCAAAGCCCGGGCGTCGGGCGACCTTTGGTCGCCCGGCCTCAGTGAGCGAGCGAGCGC
GCAGAGAGGGAGTGGCC

In some embodiments, exemplary rAAV vectors of the disclosure contain the following architecture: 5′ ITR-promoter-hFXN-ATM coding sequence-polyA-3′ ITR. In some embodiments, exemplary rAAV vectors of the disclosure contain the following architecture: 5′ ITR-promoter-hFXN-ATM coding sequence-stuffer-polyA-3′ ITR. In some embodiments, exemplary rAAV vectors of the disclosure contain the following architecture: 5′ ITR-promoter-hFXN-ATM coding sequence-WPREsf-polyA-3′ ITR. In some embodiments, exemplary rAAV vectors of the disclosure contain any of the following architectures: 5′ ITR-promoter-SV40 intron-hFXN-ATM coding sequence-polyA-stuffer-3′ ITR; 5′ ITR-promoter-SV40 intron-hFXN-ATM coding sequence-polyA-3′ ITR; 5′ ITR-promoter-SV40 intron-hFXN-ATM coding sequence-WPREsf-polyA-stuffer-3′ ITR; or 5′ ITR-promoter-SV40 intron-hFXN-ATM coding sequence-WPREsf-polyA-3′ ITR. The promoter of a vector in accordance with any of these architectures may be an hSYN1 promoter, Nefh(Ple345) promoter, Sncg promoter, Cx36 promoter, or dcx promoter. The promoter of a vector in accordance with any of these architectures may be a CBA or smCBA promoter. The polyA sequence of a vector in accordance with any of these architectures may be a bGH polyA sequence. In some embodiments, the rAAV vector of the disclosure comprises a promoter as described herein. In some embodiments, the rAAV vector of the disclosure comprises an intron as disclosed herein. In some embodiments, the rAAV vector of the disclosure comprises a sequence encoding frataxin. In some embodiments, the rAAV vector of the disclosure comprises a polyA sequence as disclosed herein. In some embodiments, the rAAV vector of the disclosure comprises a 3′ UTR comprising an ATM as disclosed herein.

In some embodiments, any of the rAAV vectors of the disclosure contain one or more stuffer sequences. In some embodiments, the rAAV vector contains a single stuffer sequence. As used herein, a “stuffer” sequence refers to a generic, inert, non-coding sequence that increases the length of the rAAV vectors. In some embodiments, the stuffer sequence increases the length of any of the disclosed rAAV vectors such that the vector is close to wtAAV genome size (˜4.8 kb). In exemplary embodiments, the stuffer is designed to bring the length of the rAAV vector between the ITRs to a length between 4.5 kB and 4.8 kB.

Accordingly, provided herein are rAAV vectors comprising a polynucleotide comprising a nucleic acid encoding a human frataxin protein and a stuffer sequence. In some embodiments, the stuffer sequence has a length of between about 1000 nucleotides and about 4000 nucleotides. In some embodiments, the stuffer sequence has a length of between about 2000 nucleotides and about 3500, about 3200, about 3000, about 2900, or about 2800 nucleotides. In some embodiments, the stuffer sequence has a length of between about 2500 nucleotides and about 3000 nucleotides. In some embodiments, the stuffer sequence has a length of about 2800 nucleotides.

In some embodiments, the AAV vector comprises a first ITR and a second ITR and the length of the AAV vector between the first ITR and the second ITR (i.e., “the length between the ITRs”) is between about 2000 nucleotides and about 6000 nucleotides. In some embodiments, such an AAV vector comprises a stuffer sequence. In some embodiments, the length of the AAV vector between the first ITR and the second ITR is between about 3000 nucleotides and about 5000 nucleotides. In some embodiments, the length of the AAV vector between the first ITR and the second ITR is between about 4000 nucleotides and about 5000 nucleotides. In some embodiments, the length of the AAV vector between the first ITR and the second ITR is between about 4500 nucleotides and about 4800 nucleotides. This length between the ITRs may be about 4500 nucleotides.

In some embodiments, the total length of the heterologous nucleic acid and the stuffer sequence is between about 2000 nucleotides and about 5000 nucleotides. In some embodiments, this total length is between about 2500 nucleotides and about 4500, about 4200, about 4000, about 3800, about 3700, about 3600, or about 3500 nucleotides. In some embodiments, the stuffer sequence has a length of about 3500 nucleotides.

In some embodiments, the stuffer sequence is positioned downstream (3′) of the heterologous nucleic acid encoding human frataxin protein. In some embodiments, the stuffer sequence is positioned upstream (5′) of the polyA sequence. In some embodiments, the stuffer sequence is positioned 3′ of the polyA sequence. In some embodiments, the stuffer sequence is positioned between two AAV ITR sequences.

In some embodiments, the polynucleotide of the nucleic acid vector comprises a polyadenylation (polyA) signal sequence. In some embodiments, the polyadenylation signal is selected from a bovine growth factor hormone (bGH) polyadenylation signal, an SV40 polyadenylation signal, a human growth factor hormone (hGH) polyadenylation signal, and a rabbit beta-globin (rbGlob) polyadenylation signal.

In some embodiments, the vector comprises a bGH polyA signal. In other embodiments, the vector comprises an SV40 polyA signal. In some embodiments, the nucleic acid vector comprises a stuffer sequence and a polyA signal. In some embodiments, the nucleic acid vector comprises one or more of the following regulatory elements: i) a WPREsf sequence, ii) a polyA signal, such as a bGH polyA signal, and iii) a stuffer sequence. In some embodiments, the nucleic acid vector comprises each of of i) a WPREsf sequence, ii) a bGH polyA signal, and iii) a stuffer sequence.

In some embodiments, the vector comprises a bGH polyA signal having a nucleic acid sequence having at least 80%, 85%, 90, 92.5%, 95%, 98% or 99% identity to SEQ ID NO: 19. In some embodiments, the polyA signal of any of the disclosed vectors comprises the nucleic acid sequence of SEQ ID NO: 19.

### Bgh Polya Signal:

(SEQ ID NO: 19)
AGCCTCGACTGTGCCTTCTAGTTGCCAGCCATCTGTTGTTTGCCCCTCC
CCCGTGCCTTCCTTGACCCTGGAAGGTGCCACTCCCACTGTCCTTTCCT
AATAAAATGAGGAAATTGCATCGCATTGTCTGAGTAGGTGTCATTCTAT
TCTGGGGGGTGGGGTGGGGCAGGACAGCAAGGGGGAGGATTGGGAAGAC
AATAGCAGGCGATAAGGATC

In some embodiments, the nucleic acid vector comprises an intron. In other embodiments, the nucleic acid vector comprises a splice donor and splice acceptor regions independent of an intron.

In some embodiments, the nucleic acid vector comprises an SV40 intron. In some embodiments, the SV40 intron comprises an SV40 splice donor region. In some embodiments, the SV40 comprises an SV40 splice acceptor region. In some embodiments, the SV40 comprises SV40 splice donor and splice acceptor regions (SV40 SD/SA) (see FIG. 5).

The length of the SV40 intron is 99 nucleotides. It was first reported by Ostedgaard et al. that the presence of the SV40 intron between the promoter and the transgene in an AAV expression cassette provided a two-fold increase of transgene expression in lung carcinoma cells, while under the control of a CMV promoter and enhancer (PNAS 2005; 102(8): 2952-2957). Recently, it was shown that positioning an SV40 intron downstream of the expression cassette in a non-viral vector resulted in highest levels of expression of a reporter transgene (in Chinese hamster ovary cells). See Xu et al., J. Cell. Mol. Med. Vol 22, No 4 (2018): 2231-2239. In some embodiments of the disclosed rAAV vectors, the SV40 intron is positioned downstream (3′) of the heterologous nucleic acid. In other embodiments, the SV40 intron is positioned upstream (5′) of the heterologous nucleic acid.

In some embodiments, the nucleic acid vector comprising an SV40 intron has a length of between 1700 nucleotides (nt, or base pairs (bp)) and about 3500, about 3200, about 3000, about 2900, or about 2800 nucleotides. In some embodiments, the nucleic acid vector comprising an SV40 intron (or an SV40 splice acceptor and splice donor sequence) has a length of between 2000 nucleotides and about 3500, about 3200, about 3000, about 2900, or about 2800 nucleotides. In some embodiments, the nucleic acid vector has a length of about 2614 nucleotides (e.g., the vector of FIG. 5). In some embodiments, the nucleic acid vector has a length of about 6443 nucleotides (e.g., the vector of FIG. 20). In some embodiments, the nucleic acid vector has a length of about 5393 nucleotides (e.g., the vector of FIG. 21).

### Capsid Variants

As described in the Examples herein, it was found that AAV2 capsid variants P2-V1 and P2-V1(Y-F+T-V) mediated improved RGC transduction relative to benchmark capsids (e.g., AAV2 vectors) in macaques. Accordingly, the disclosure provides rAAV particles comprising a capsid protein of the P2-V1 serotype and the and P2-V1(Y-F+T-V) serotype, and related compositions and methods. In some embodiments, the rAAV particle comprises a heterologous nucleic acid, e.g., encoding a therapeutic or diagnostic agent. The heterologous nucleic acid may be in the form of a single-stranded (ss) or self-complementary (sc) AAV nucleic acid vector, such as single-stranded or self-complementary recombinant viral genome.

In some embodiments, the capsid comprises AAV2, AAV6, or a capsid variant derived from AAV2 or AAV6. Accordingly, in some embodiments, the capsid comprises the sequence of SEQ ID NO: 32 or SEQ ID NO: 6, respectively. In some embodiments, the capsid protein comprises a capsid variant of serotype 2 (AAV2) selected from P2-V1 (also known as ME-B), P2-V1(Y-F+T-V) (also known as ME-B(Y-F+T-V)), P2-V2, P2-V3, and DGE-DF. The DGE-DF capsid variant contains aspartic acid, glycine, glutamic acid, aspartic acid, and phenylalanine at positions 492, 493, 494, 499, and 500 of the AAV2 VP1 sequence. In some embodiments, the capsid comprises a capsid variant of serotype 2 (AAV2) selected from AAV2(7m8), AAV-DJ, AAV2/2-MAX, AAV2G9, or elements thereof.

In some embodiments, the capsid comprises AAV3, AAV5, AAV8, or a capsid variant derived from AAV3, AAV5, or AAV8. In some embodiments, the capsid is selected from AAV3, AAV3b, AAVLK03, AAV7BP2, AAV5, AAV1(E531K), AAVSHh10, AAVSHh10Y, AAV6(D532N), AAV6-3pMut, AAV8, and AAV8(Y447F+Y733F+T494V) (SEQ ID NO: 38).

The disclosure further provides rAAV particles having AAV44.9 capsids or capsid variants thereof. The disclosure further provides rAAV particles having AAV44.9 capsids that comprise the E531D substitition and one or more additional substitutions, such as a Y-F mutation at residue 446, a T-V mutation at residue 492, or both. The amino acid sequence of the AAV44.9(E531D) capsid is provided at SEQ ID NO: 40, below. Accordingly, the disclosure provides rAAV particles comprising an AAV44.9(T492V+E531D) capsid, an AAV44.9(Y446F+E531D) capsid, or an AAV44.9(Y446F+T492V+E531D) capsid, and related compositions and methods.

In some embodiments, the disclosure provides rAAV particles comprising an AAV2(4pMut)ΔHS capsid (SEQ ID NO: 37). The AAV2(4pMut)ΔHS capsid was shown to mediate enhanced lateral spread in primate retina and display efficient photoreceptor transduction. The disclosure also provides particles comprising AAV8(Y733F) and AAV8(Y447F+Y733F+T494V) capsid variants.

Aspects of this disclosure relate to vectors comprising an AAV44.9(E531D) capsid that exhibits enhanced lateral spread after subretinal injection to the fovea, wherein detachment of the fovea (e.g., a temporary bullous detachment) is minimized. In some embodiments, the disclosure provides a capsid protein, e.g., a VP1, VP2 or VP3 capsid protein, in accordance with the sequence of the VP1, VP2 and VP3 of the P2-V1 capsid (see International Patent Publication No. WO 2018/156654, wherein the capsids are herein incorporated by reference) The VP1 amino acid sequence is reproduced below.

In some embodiments, the disclosure provides an rAAV particle comprising a capsid comprising a VP1, VP2, and/or VP3 protein, wherein the rAAV particle further comprises a polynucleotide comprising a heterologous nucleic acid. In some embodiments, the rAAV particle comprises a P2-V1 or P2-V1(Y-F+T-V) capsid, and wherein the AAV further comprises a polynucleotide comprising a heterologous nucleic acid. The polynucleotide containing the heterologous nucleic acid may be flanked by one or more inverted terminal repeat (ITR) sequences.

In some embodiments, the disclosure provides a capsid protein comprising an amino acid sequence having at least 80% identity, at least 85% identity, at least 90% identity, at least 92.5% identity, at least 95% identity, 98% identity, or 99% identity to any of SEQ ID NOs: 1 or 2. In some embodiments, the disclosure provides a capsid protein comprising the amino acid sequence of SEQ ID NO: 1 or 2. In particular embodiments, capsids comprising the amino acid sequence set forth as SEQ ID NO: 1 or 2 are provided. In some embodiments, the disclosed capsids may comprise a sequence having 1, 2, 3, 4, 5, 6, 7, 8, 9, 10, 11, 12, 13, 14, 15, 16, 17, or 18 amino acids that differ relative to the sequence of SEQ ID NOs: 1 or 2. These differences may comprise amino acids that have been inserted, deleted, or substituted relative to the sequence of SEQ ID NOs: 1 or 2.

In some embodiments, the disclosure provides a capsid protein comprising an amino acid sequence having at least 80% identity, at least 85% identity, at least 90% identity, at least 92.5% identity, at least 95% identity, 98% identity, or 99% identity to any of SEQ ID NOs: 6 and 32-40. In some embodiments, the disclosure provides a capsid protein comprising the amino acid sequence of any one of SEQ ID NO: 6 and 32-40. In some embodiments, the disclosed capsids may comprise a sequence having 1, 2, 3, 4, 5, 6, 7, 8, 9, 10, 11, 12, 13, 14, 15, 16, 17, or 18 amino acids that differ relative to the sequence of SEQ ID NOs: 6 and 32-40. These differences may comprise amino acids that have been inserted, deleted, or substituted relative to the sequence of SEQ ID NOs: 6 and 32-40.

In some embodiments, the viral vector is a recombinant adeno-associated viral (rAAV) vector. In some embodiments, the rAAV vector is self-complementary. In some embodiments, the nucleic acid is comprised within a cell, e.g., a mammalian or insect cell.

The P2-V1 capsid protein is set forth as SEQ ID NO: 1 below. All substitutions in the P2-V1 capsid protein as described herein are based on the VP1 amino acid sequence set forth in SEQ ID NO: 1. The P2-V1(Y-F+T-V) capsid variant contains aspartic acid, glycine, glutamic acid, aspartic acid, and phenylalanine at positions 492, 493, 494, 499, and 500 of the AAV2 VP1 sequence, respectively, SAAGADXAXDS (SEQ ID NO: 4) at positions 546-556 of AAV2 VP1, and the following substitutions: Y272F, Y444F, and T491V. The P2-V1(Y-F+T-V) VP1 amino acid sequence is set forth as SEQ ID NO: 2. As would be appreciated by one of skill in the art, all substitutions described herein in the VP1 sequence are equally applicable to the sequences of the VP2 and VP3 proteins. The sequences of SEQ ID NOs: 1 and 2 are provided below.

### Table 1.

### >AAV2

(SEQ ID NO: 32)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDINGVYSEPRPIGTRYLTRN
L*

### >AAB2(7m8)

(SEQ ID NO: 33)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVTTTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNL
ALGETTRPARQAATADVNTQGVLPGMVWQDRDVYLQGPIWAKIPHTDGH
FHPSPLMGGFGLKHPPPQILIKNTPVPANPSTTFSAAKFASFITQYSTG
QVSVEIEWELQKENSKRWNPEIQYTSNYNKSINVDFTVDINGVYSEPRP
IGTRYLTRNL*

### >AAV2 DGE-DF

(SEQ ID NO: 34)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TDGENNNSDFSWTGATKYHLNGRDSLVNPGPAMASHKDDEEKFFPQSGV
LIFGKQGSEKTNVDIEKVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDTNGVYSEPRPIGTRYLTRN
L*

### >AAV2 P2-V1

(SEQ ID NO: 1)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSNAGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TDGENNNSDFSWTGATKYHLNGRDSLVNPGPAMASHKDDEEKFFPQSGV
LIFGKQSAAGADVAIDSVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDTNGVYSEPRPIGTRYLTRN
L*

### >AAV2 P2-V2

(SEQ ID NO: 35)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSASGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVST
TPADNNNSDFSWTGATKYHLNGRDSLVNPGPAMASHKDDDEKFFPQSGV
LIFGKQGSEKTNVDIEKVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDTNGVYSEPRPIGTRYLTRN
L*

### >AAV2 P2-V3

(SEQ ID NO: 36)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSNAGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYFLSRTNTASGNVTQSTLQFSQAGASDIRDQSRNWLPGPCYRQQRVSK
TSADNNNSEYSWTGATKYHLNGRDSLVNPGPAMASHRDDDDKFFPQSGV
LIFGKQGSEKTNVDIEKVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDTNGVYSEPRPIGTRYLTRN
L*

AAV2 4pMut(ΔHS)

(SEQ ID NO: 37)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYFLSRTNTPSGTTTQSRLQFSQAGASDIRDQSRNWLPGPCYRQQGVSK
VSADNNNSEFSWTGATKYHLNGRDSLVNPGPAMASHKDDEEKFFPQSGV
LIFGKQGSEKTNVDIEKVMITDEEEIRTTNPVATEQYGSVSTNLQSGNT
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDTNGVYSEPRPIGTRFLTRN
L*

### >AAV8 (Y447F+Y733F+T494V)

(SEQ ID NO: 38)
MAADGYLPDWLEDNLSEGIREWWALKPGAPKPKANQQKQDDGRGLVLPG
YKYLGPFNGLDKGEPVNAADAAALEHDKAYDQQLQAGDNPYLRYNHADA
EFQERLQEDTSFGGNLGRAVFQAKKRVLEPLGLVEEGAKTAPGKKRPVE
PSPQRSPDSSTGIGKKGQQPARKRLNFGQTGDSESVPDPQPLGEPPAAP
SGVGPNTMAAGGGAPMADNNEGADGVGSSSGNWHCDSTWLGDRVITTST
RTWALPTYNNHLYKQISNGTSGGATNDNTYFGYSTPWGYFDFNRFHCHF
SPRDWQRLINNNWGFRPKRLSFKLFNIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFQFTYTFEDVPFHSSYAHSQSLDRLMNPLI
DQYLYFLSRTQTTGGTANTQTLGFSQGGPNTMANQAKNWLPGPCYRQQR
VSTVTGQNNNSNFAWTAGTKYHLNGRNSLANPGIAMATHKDDEERFFPS
NGILIFGKQNAARDNADYSDVMLTSEEEIKTTNPVATEEYGIVADNLQQ
QNTAPQIGTVNSQGALPGMVWQNRDVYLQGPIWAKIPHTDGNFHPSPLM
GGFGLKHPPPQILIKNTPVPADPPTTFNQSKLNSFITQYSTGQVSVEIE
WELQKENSKRWNPEIQYTSNYYKSTSVDFAVNTEGVYSEPRPIGTRFLT
RNL*

### >AAV5

(SEQ ID NO: 39)
MSFVDHPPDWLEEVGEGLREFLGLEAGPPKPKPNQQHQDQARGLVLPGY
NYLGPGNGLDRGEPVNRADEVAREHDISYNEQLEAGDNPYLKYNHADAE
FQEKLADDTSFGGNLGKAVFQAKKRVLEPFGLVEEGAKTAPTGKRIDDH
FPKRKKARTEEDSKPSTSSDAEAGPSGSQQLQIPAQPASSLGADTMSAG
GGGPLGDNNQGADGVGNASGDWHCDSTWMGDRVVTKSTRTWVLPSYNNH
QYREIKSGSVDGSNANAYFGYSTPWGYFDFNRFHSHWSPRDWQRLINNY
WGFRPRSLRVKIFNIQVKEVTVQDSTTTIANNLTSTVQVFTDDDYQLPY
VVGNGTEGCLPAFPPQVFTLPQYGYATLNRDNTENPTERSSFFCLEYFP
SKMLRTGNNFEFTYNFEEVPFHSSFAPSQNLFKLANPLVDQYLYRFVST
NNTGGVQFNKNLAGRYANTYKNWFPGPMGRTQGWNLGSGVNRASVSAFA
TTNRMELEGASYQVPPQPNGMTNNLQGSNTYALENTMIFNSQPANPGTT
ATYLEGNMLITSESETQPVNRVAYNVGGQMATNNQSSTTAPATGTYNLQ
EIVPGSVWMERDVYLOGPIWAKIPETGAHFHPSPAMGGFGLKHPPPMML
IKNTPVPGNITSFSDVPVSSFITQYSTGQVTVEMEWELKKENSKRWNPE
IQYTNNYNDPQFVDFAPDSTGEYRTTRPIGTRYLTRPL*

### >AAV44.9(E531D)

(SEQ ID NO: 40)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAVFQAKKRVLEPLGLVEEGAKTAPGKKRPVE
QSPQEPDSSSGIGKTGQQPAKKRLNFGQTGDTESVPDPQPLGEPPAAPS
GLGPNTMASGGGAPMADNNEGADGVGNSSGNWHCDSTWLGDRVITTSTR
TWALPTYNNHLYKQISNGTSGGSTNDNTYFGYSTPWGYFDFNRFHCHFS
PRDWQRLINNNWGFRPKRLNFKLFNIQVKEVTTNEGTKTIANNLTSTVQ
VFTDSEYQLPYVLGSAHQGCLPPFPADVFMVPQYGYLTLNNGSQALGRS
SFYCLEYFPSQMLRTGNNFQFSYTFEDVPFHSSYAHSQSLDRLMNPLID
QYLYYLVRTQTTGTGGTQTLAFSQAGPSSMASQARNWVPGPSYRQQRVS
TTTNQNNNSNFAWTGAAKFKLNGRDSLMNPGVAMASHKDDDDRFFPSSG
VLIFGKQGAGNDGVDYSQVLITDEEEIKATNPVATEEYGAVAINNQAAN
TQAQTGLVHNQGVIPGMVWQNRDVYLQGPIWAKIPHTDGNFHPSPLMGG
FGLKHPPPQILIKNTPVPADPPLTFNQAKLNSFITQYSTGQVSVEIEWE
LQKENSKRWNPEIQYTSNYYKSTNVDFAVNTEGVYSEPRPIGTRYLTRN
L*

### >P2-V1(Y-F+T-V)

(SEQ ID NO: 2)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSNAGASNDNHFFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYFLSRTNTPSGTTTQSRLQFSQAGASDIRDQSRNWLPGPCYRQQRVSK
VDGENNNSDFSWTGATKYHLNGRDSLVNPGPAMASHKDDEEKFFPQSGV
LIFGKQSAAGADVAIDSVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDINGVYSEPRPIGTRFLTRNL

### >AAV6

(SEQ ID NO: 6)
MAADGYLPDWLEDNLSEGIREWWDLKPGAPKPKANQQKQDDGRGLVLPG
YKYLGPFNGLDKGEPVNAADAAALEHDKAYDQQLKAGDNPYLRYNHADA
EFQERLQEDTSFGGNLGRAVFQAKKRVLEPFGLVEEGAKTAPGKKRPVE
QSPQEPDSSSGIGKTGQQPAKKRLNFGQTGDSESVPDPQPLGEPPATPA
AVGPTTMASGGGAPMADNNEGADGVGNASGNWHCDSTWLGDRVITTSTR
TWALPTYNNHLYKQISSASTGASNDNHYFGYSTPWGYFDFNRFHCHFSP
RDWQRLINNNWGFRPKRLNFKLFNIQVKEVTTNDGVTTIANNLTSTVQV
FSDSEYQLPYVLGSAHQGCLPPFPADVFMIPQYGYLTLNNGSQAVGRSS
FYCLEYFPSQMLRTGNNFTFSYTFEDVPFHSSYAHSQSLDRLMNPLIDQ
YLYYLNRTQNQSGSAQNKDLLFSRGSPAGMSVQPKNWLPGPCYRQQRVS
KTKTDNNNSNFTWTGASKYNLNGRESIINPGTAMASHKDDKDKFFPMSG
VMIFGKESAGASNTALDNVMITDEEEIKATNPVATERFGTVAVNLQSSS
TDPATGDVHVMGALPGMVWQDRDVYLQGPIWAKIPHTDGHFHPSPLMGG
FGLKHPPPQILIKNTPVPANPPAEFSATKFASFITQYSTGQVSVEIEWE
LQKENSKRWNPEVQYTSNYAKSANVDFTVDNNGLYTEPRPIGTRYLTRP
L

In some embodiments, an AAV-DJ capsid is used in conjunction with the rAAV vectors of the disclosure. AAV-DJ comprises the insertion of 7 amino acids into the HSPG binding domain of the AAV2 capsid and has high expression efficiency in Muller cells following intravitreal injection. In some embodiments, an AAV2(7m8) is used in conjunction with the rAAV vectors of the disclosure. The AAV2(7m8) capsid is closely related to AAV-DJ. In some embodiments, the AAV2/2-MAX capsid comprises five point mutations, Y272F, Y444F, Y500F, Y730F, T491V. In some embodiments, the AAVSHh10 and AAV6(D532N) capsids are derivatives of AAV6. In some embodiments, the AAV6-3pMut is (also known as AAV6(TM6) and AAV6(Y705+Y731F+T492V)).

In some embodiments, the capsid used in conjunction with the disclosed rAAV vectors is a capsid comprising non-native amino acid substitutions at amino acid residues of a wild-type AAV2 capsid. In some embodiments, the non-native amino acid substitutions comprise one or more of Y272F, Y444F, T491V, Y500F, Y700F, Y704F, Y730F or a combination thereof. In some embodiments, the capsids comprises non-native amino acid substitutions at amino acid residues of a wild-type AAV6 capsid as set forth in SEQ ID NO: 6. In some embodiments, the non-native amino acid substitutions comprise one or more of Y445F, Y705F, Y731F, T492V, S663V or a combination thereof. In some embodiments, the capsid comprises AAV2G9, a variant of AAV2.

In some embodiments, the capsid comprises a non-native amino acid substitution at amino acid residue 533 of a wild-type AAV8 capsid. In some embodiments, the non-native amino acid substitution is E533K, Y733F, or a combination thereof. In some embodiments, the capsid comprises AAV7BP2, a variant of AAV8.

In some embodiments, the capsid comprises non-native amino acid substitutions of a wild-type AAV2 capsid. In some embodiments, the capsid comprises one or more of:
(a) Y444F;
(b) Y444F+Y500F+Y730F;
(c) Y272F+Y444F+Y500F+Y730F;
(d) Y444F+Y500F+Y730F+T491V; or
(e) Y272F+Y444F+Y500F+Y730F+T491V, or at equivalent amino acid positions corresponding thereto in any one of the wild-type AAV1, AAV3, AAV4, AAV5, AAV7, AAV9, or AAV10 capsid proteins.

In some embodiments, the capsid comprises non-native amino acid substitutions of a wild-type AAV6 capsid. In some embodiments, the capsid comprises one or more of:
(a) Y445F;
(b) Y705F+Y731F;
(c) T492V;
(d) Y705F+Y731F+T492V;
(e) S663V; or
(f) S663V+T492V.

In various embodiments, the rAAV particles comprise one of the following capsids, i.e., capsid variants of AAV2: DGE-DF (also known as ‘V1V4 VR-V’), P2-V2, and P2-V3. The DGE-DF capsid variant contains aspartic acid, glycine, glutamic acid, aspartic acid, and phenylalanine at amino acid positions 492, 493, 494, 499, and 500 of wild-type AAV2 VP1. The P2-V2 capsid variant contains alanine, threonine, proline, aspartic acid, phenylalanine, and aspartic acid at positions 263, 490, 492, 499, 500, and 530 of AAV2 VP1. The P2-V3 capsid variant contains asparagine, alanine, phenylalanine, alanine, asparagine, valine, threonine, arginine, aspartic acid, and aspartic acid at positions 263, 264, 444, 451, 454, 455, 459, 527, 530, and 531 of AAV2 VP1. In some embodiments, the capsid comprises the sequence of SEQ ID NO: 33 (AAV2(7m8)), NO: 34 (DGE-DF), NO: 35 (P2-V2), or NO: 36 (P2-V3). In other embodiments, the rAAV particles comprise a capsid selected from AAV6-3pMut, AAV2(quadY-F+T-V), or AAV2(trpY-F). In some embodiments, the rAAV particles comprise any of the capsid variants described in International Patent Publication No. WO 2018/156654, the disclosures of these capsid variants being incorporated by reference herein.

### Vectors

Recombinant AAV vectors are described herein. In some embodiments, the vector described herein is a self-complementary rAAV (scAAV) vector. In some embodiments, the vector is a single-stranded (ss) vector. In some embodiments, the vector is provided to the one or both eyes by one or more administrations of an infectious adeno-associated viral particle, an rAAV virion, or a plurality of any of the disclosed rAAV particles in an amount and for a time sufficient to treat or ameliorate one or more symptoms of the disease or condition being treated. In some embodiments, any of the disclosed rAAV particles is provided to the one or both eyes in any of the disclosed pharmaceutical compositions, e.g., a pharmaceutical composition comprising one or more pharmaceutically acceptable excipients.

In some aspects, a method for providing a mammal in need thereof with a therapeutically-effective amount of a selected therapeutic agent (e.g., a synthetic human FXN) is described herein. In some embodiments, the therapeutic agent is encoded in a heterologous nucleic acid, or transgene, that is inserted into a recombinant AAV nucleic acid vector. In some embodiments, the nucleic acid vector comprises a polynucleotide containing a heterologous nucleic acid comprising a sequence encoding a frataxin-encoding polypeptide of interest operably linked to a promoter (e.g., an hSYN1 promoter, or other RGC promoter such as Nefh(Ple345) (e.g., human and mouse), Sncg (e.g., human and mouse), Cx36 (e.g., human), or dcx (e.g., human)), wherein the polynucleotide is flanked on each side with an ITR sequence. The disclosed nucleic acid vectors may comprise AAV inverted terminal repeats flanking a polynucleotide comprising the FXN heterologous nucleic acid (transgene), 3′ UTR, and regulatory elements. In some embodiments, the disclosed nucleic acid vectors comprise AAV ITRs flanking a polynucleotide comprising the FXN heterologous nucleic acid, SV40 intron, WPREsf element, and polyA signal sequence. In some embodiments, the vectors comprise AAV ITRs flanking a polynucleotide comprising the FXN heterologous nucleic acid, SV40 intron, WPREsf element, stuff sequence, and polyA sequence.

The ITR sequences can be derived from any AAV serotype (e.g., 1, 2, 3, 4, 5, 6, 7, 8, 9, or 10) or can be derived from more than one serotype. In some embodiments, the ITR sequences of the first serotype are derived from AAV2, AAV5, AAV7 AAV8, or AAV9. In some embodiments, the ITR sequences are derived from AAV2 or AAV5. In some embodiments, the ITR sequences are the same serotype as the capsid (e.g., AAV5 ITR sequences and AAV5 capsid, etc.).

In exemplary embodiments, the ITR sequences are derived from AAV serotype 2 (AAV2). Any of the disclosed rAAV vectors may comprise a 5′ ITR (or left ITR) sequence comprising SEQ ID NO: 29 and a 3′ ITR (or right ITR) sequence comprising SEQ ID NO: 30.

### Left ITR Sequence (SEQ ID NO: 29):

GGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGC
GAGCGCGCAGAGAGGGAGTGGCCAACTCCATCACTAGGGGTTCCT

### Right ITR Sequence (SEQ ID NO: 30):

AGGAACCCCTAGTGATGGAGTTGGCCACTCCCTCTCTGCGCGCTCGCTC
GCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGCC

ITR sequences and plasmids containing ITR sequences are known in the art and commercially available (see, e.g., products and services available from Vector Biolabs, Philadelphia, PA; Cellbiolabs, San Diego, CA; Agilent Technologies, Santa Clara, Ca; and Addgene, Cambridge, MA; and Gene delivery to skeletal muscle results in sustained expression and systemic delivery of a therapeutic protein. In some embodiments, the nucleic acid vector comprises a pTR-UF-11 plasmid backbone, which is a plasmid that contains AAV2 ITRs. This plasmid is commercially available from the American Type Culture Collection (ATCC MBA-331).

In some embodiments, the disclosure provides improved rAAV particles that have been derived from a number of different serotypes, including but not limited to AAV1, AAV2, AAV3, AAV4, AAV5, AAV6, AAV7, AAV8, AAV9, AAV10 and combinations thereof. In some embodiments, the capsid comprises AAV5 or AAV44.9(E531D). In some embodiments, the capsid may comprise a variant of AAV5, a variant of AAV7, a variant of AAV8, or a variant of AAV9. In some embodiments, the capsid comprises any of AAV2(4pMut)ΔHS, AAV44.9, AAVrh.8, AAVrh.8R, AAVrh.10, AAVrh.74, AAV2TT, AAV2HBKO, AAV8(Y447F+Y733F+T494V), or AAVAnc80.

In some aspects, the rAAV vectors described herein may comprise multiple (two, three, four, five, six, seven, eight, nine, or ten) heterologous nucleic acids. In certain embodiments, the multiple heterologous nucleic acids are comprised on a single polynucleotide molecule. Multiple heterologous nucleic acids may be used, for example, to correct or ameliorate a gene defect caused by a multi-subunit protein. In various embodiments, a different heterologous nucleic acid may be used to encode each subunit of a protein, or to encode different peptides or proteins. This is desirable when the size of the nucleic acid encoding the protein subunit is large, e.g., for an immunoglobulin, the platelet-derived growth factor, or a dystrophin protein. In order for the cell to produce the multi-subunit protein, a cell is infected with the rAAV particle containing each of the different subunits. Alternatively, different subunits of a protein may be encoded by the same nucleic acid sequence. In various embodiments, a single heterologous nucleic acid includes the nucleic acid encoding each of the subunits, with the nucleic acid for each subunit separated by an internal ribozyme entry site (IRES). This is desirable when the size of the nucleic acid encoding each of the subunits is small, e.g., the total size of the nucleic acid encoding the subunits and the IRES is less than five kilobases.

As an alternative to an IRES, the nucleic acid may be separated by sequences encoding a 2A peptide, which self-cleaves in a post-translational event. This 2A peptide is significantly smaller than an IRES, making it well suited for use when space is a limiting factor. More often, when the heterologous nucleic acid is large, consists of multi-subunits, or two heterologous nucleic acids are co-delivered, or rAAV particle carrying the desired heterologous nucleic acid(s) or subunits are co-administered to allow them to concatamerize in vivo to form a single vector genome. In such an embodiment, a first rAAV particle may carry an expression cassette which expresses a single heterologous nucleic acid and a second rAAV particle may carry an expression cassette which expresses a different heterologous nucleic acid for co-expression in the host cell. However, the selected heterologous nucleic acid may encode any biologically active product or other product, e.g., a product desirable for study.

In some aspects, the rAAV vectors may be codon-optimized for mammalian expression. In some aspects, the rAAV vectors may be codon-optimized for human expression. In some embodiments, the rAAV vectors may have modified Kozak nucleic acid sequences that provide for enhanced transduction or fitness in the target cell, e.g., an RGC. Kozak sequences include the translation initiation codon (ATG) and a stretch of nucleotides positioned 5′ of the initiation codon.

### Exemplary Human FXN 3′ UTR

(SEQ ID NO: 9)
TCCCCAGCCCCGTTTTAAGGACATTAAAAGCTATCAGGCCAAGACCCCA
GCTTCATTATGCAGCTGAGGTCTGTTTTTTGTTGTTGTTGTTGTTTATT
TTTTTTATTCCTGCTTTTGAGGACAGTTGGGCTATGTGTCACAGCTCTG
TAGAAAGAATGTGTTGCCTCCTACCTTGCCCCCAAGTTCTGATTTTTAA
TTTCTATGGAAGATTTTTTGGATTGTCGGATTTCCTCCCTCACATGATA
CCCCTTATCTTTTATAATGTCTTATGCCTATACCTGAATATAACAACCT
TTAAAAAAGCAAAATAATAAGAAGGAAAAATTCCAGGAGGGAAAATGAA
TTGTCTTCACTCTTCATTCTTTGAAGGATTTACTGCAAGAAGTACATGA
AGAGCAGCTGGTCAACCTGCTCACTGTTCTATCTCCAAATGAGACACAT
TAAAGGGTAGCCTACAAATGTTTTCAGGCTTCTTTCAAAGTGTAAGCAC
TTCTGAGCTCTTTAGCATTGAAGTGTCGAAAGCAACTCACACGGGAAGA
TCATTTCTTATTTGTGCTCTGTGACTGCCAAGGTGTGGCCTGCACTGGG
TTGTCCAGGGAGACCTAGTGCTGTTTCTCCCACATATTCACATACGTGT
CTGTGTGTATATATATTTTTTCAATTTAAAGGTTAGTATGGAATCAGCT
GCTACAAGAATGCAAAAAATCTTCCAAAGACAAGAAAAGAGGAAAAAAA
GCCGTTTTCATGAGCTGAGTGATGTAGCGTAACAAACAAAATCATGGAG
CTGAGGAGGTGCCTTGTAAACATGAAGGGGCAGATAAAGGAAGGAGATA
CTCATGTTGATAAAGAGAGCCCTGGTCCTAGACATAGTTCAGCCACAAA
GTAGTTGTCCCTTTGTGGACAAGTTTCCCAAATTCCCTGGACCTCTGCT
TCCCCATCTGTTAAATGAGAGAATAGAGTATGGTTGATTCCCAGCATTC
AGTGGTCCTGTCAAGCAACCTAACAGGCTAGTTCTAATTCCCTATTGGG
TAGATGAGGGGATGACAAAGAACAGTTTTTAAGCTATATAGGAAACATT
GTTATTGGTGTTGCCCTATCGTGATTTCAGTTGAATTCATGTGAAAATA
ATAGCCATCCTTGGCCTGGCGCGGTGGCTCACACCTGTAATCCCAGCAC
TTTTGGAGGCCAAGGTGGGTGGATCACCTGAGGTCAGGAGTTCAAGACC
AGCCTGGCCAACATGATGAAACCCCGTCTCTACTAAAAATACAAAAAAT
TAGCCGGGCATGATGGCAGGTGCCTGTAATCCCAGCTACTTGGGAGGCT
GAAGCGGAAGAATCGCTTGAACCCAGAGGTGGAGGTTGCAGTGAGCCGA
GATCGTGCCATTGCACTGTAACCTGGGTGACTGAGCAAAACTCTGTCTC
AAAATAATAATAACAATATAATAATAATAATAGCCATCCTTTATTGTAC
CCTTACTGGGTTAATCGTATTATACCACATTACCTCATTTTAATTTTTA
CTGACCTGCACTTTATACAAAGCAACAAGCCTCCAGGACATTAAAATTC
ATGCAAAGTTATGCTCATGTTATATTATTTTCTTACTTAAAGAAGGATT
TATTAGTGGCTGGGCATGGTGGCGTGCACCTGTAATCCCAGGTACTCAG
GAGGCTGAGACGGGAGAATTGCTTGACCCCAGGCGGAGGAGGTTACAGT
GAGTCGAGATCGTACCTGAGCGACAGAGCGAGACTCCGTCTCAAAAAAA
AAAAAAAGGAGGGTTTATTAATGAGAAGTTTG

### Methods of Treatment and Formulations

Aspects of the disclosure relate to treatment of Friedreich's ataxia. In some embodiments, the method comprises administering a therapeutically effective amount of an rAAV particle or a composition as described herein to a subject having Friedreich's ataxia.

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease, e.g., Friedreich's ataxia. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, methods are provided involving providing a mammal in need thereof with a therapeutically effective amount of a selected therapeutic agent, the method comprising administering to one or both eyes of the mammal, an amount of the rAAV particles described herein; and for a time effective to provide the mammal with a therapeutically-effective amount of the selected therapeutic agent. In certain embodiments, the mammal is suspected of having, is at risk for developing, or has been diagnosed with Friedreich's ataxia.

In some embodiments, methods are provided for transducing a photoreceptor cell or a mammalian retinal ganglion cell (RGC), the method comprising administering to one or both eyes of a mammal any of the rAAV particles or any of the compositions described herein. In particular embodiments, methods are provided for expressing a polynucleotide in one or more RGCs of a mammal, the method comprising subretinally or intravitreally administering to one or both eyes of the mammal the rAAV particles described herein, or compositions thereof, wherein the rAAV particle comprises a polynucleotide comprising at least a first polynucleotide that comprises an RGC-cell-specific promoter (or otherwise a promoter specific to retinal tissue or nervous tissue) operably linked to at least a first heterologous nucleic acid sequence that encodes a therapeutic agent, for a time effective to produce the therapeutic agent in the one or more RGC cells of the mammal.

In particular embodiments, a replacement coding sequence is administered to the subject to provide a functional protein, e.g., FXN protein, to restore, e.g., completely or partially, photoreceptor function to a subject (e.g., a human). In some embodiments, one or both alleles of a target coding sequence of the subject are silenced by administering an rAAV particle comprising a heterologous nucleic acid disclosed herein to the subject (e.g., to a human having dominant cone-rod dystrophy). In particular embodiments, the endogenous mutant alleles of one or more target coding sequences are silenced or suppressed by administering an rAAV particle disclosed herein.

In some embodiments, the mammal is a human subject. In some embodiments, the mammal is a non-human primate subject. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. Other exemplary subjects include domesticated animals such as dogs and cats; livestock such as horses, cattle, pigs, sheep, goats, and chickens; and other animals such as mice, rats, guinea pigs, and hamsters.

In certain embodiments, methods are provided for subretinally administering to a fovea (e.g., foveal cone cells) of the mammal the rAAV particles described herein or compositions thereof. In particular embodiments, detachment of the fovea is minimized during and/or after subretinal administration. In particular embodiments, subretinal administration of the rAAV particle is performed in the absence of any detachment of the fovea.

In some embodiments, rAAV particles are administered via methods described herein. In some embodiments, rAAV particles are administered subretinally. In some embodiments, rAAV particles are administered intravitreally.

In some embodiments, the disclosure provides formulations of one or more rAAV-based compositions disclosed herein in pharmaceutically acceptable solutions for administration to a cell or an animal, either alone or in combination with one or more other modalities of therapy, and in particular, for therapy of human cells, tissues, and diseases affecting man.

The rAAV particles described herein may be administered in combination with other agents as well, such as, e.g., proteins or polypeptides or various pharmaceutically-active agents, including one or more systemic or topical administrations of therapeutic polypeptides, biologically active fragments, or variants thereof. In particular embodiments, the described rAAV particles may be administered in combination with one or more carbonic anhydrase inhibitors (CAIs). In some embodiments, they may be co-administered with any of the CAIs acetazolamide, dichlorphenamide (also known as diclofenamide), methazolamide, dorzolamide, brinzolamide, ethoxzolamide, and zonisamide. There is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized as described herein.

In some embodiments, described herein are uses of the described rAAV vectors, viral particles, compositions, and (host) cells described herein in the preparation of medicaments for diagnosing, preventing, treating or ameliorating at least one or more symptoms of FA. In some embodiments, the methods comprise direct administration to the vitreous of one or both eyes of a mammal in need thereof, one or more of the described vectors, viral particles, cells, compositions, or pluralities thereof, in an amount and for a time sufficient to diagnose, prevent, treat, or lessen one or more symptoms of such a disease, dysfunction, disorder, abnormal condition, deficiency, injury, or trauma in one or both eyes of the affected mammal. In various embodiments, the mammal is a human subject.

In some embodiments, described herein are compositions useful in treating FA in the preparation of medicaments to treat FA, comprising one or more of the described rAAV vectors, particles, compositions, and host cells. In some embodiments, the compositions comprise at least a first pharmaceutically-acceptable excipient for use in the manufacture of medicaments and methods involving therapeutic administration of such rAAV particles or vectors. In some embodiments, pharmaceutical formulations are suitable for intravitreal administration into one or both eyes of a human or other mammal.

In some embodiments, described herein are methods and uses of the described rAAV vectors and compositions for treating or ameliorating the symptoms of FA in human photoreceptor cells (e.g., RGCs). In some embodiments, the disclosed methods and uses comprise intravitreal or subretinal administration to one or both eyes of a subject in need thereof, one or more of the described particles vectors, particles, host cells, or compositions, in an amount and for a time sufficient to treat or ameliorate the symptoms of such a deficiency in the affected mammal. In some embodiments, the methods comprise prophylactic treatment of an animals suspected of having such conditions, or administration of such compositions to those animals at risk for developing such conditions either following diagnosis, or prior to the onset of symptoms.

In some aspects, combination therapies involving the administration of multiple rAAV vectors are contemplated. In some embodiments of the disclosed methods of treatment, the methods further comprising administering intravenously, intrathecally, or intracisternally to a subject (e.g., a subject suffering from FA symptoms in the CNS or the PNS) a second rAAV particle comprising a second rAAV vector that comprises a human FXN coding sequence. In some embodiments, the second rAAV vectors comprises an AAV9-CBA-FXN vector. In some embodiments, the second rAAV vectors comprises an AAV9-desmin-FXN vector (see, e.g., the vectors disclosed in US Publication No. 2018/0117178). In some embodiments, the second rAAV particle is administered intrathecally.

In some embodiments, the second rAAV particle is administered after the first rAAV particle. In some embodiments, the second rAAV particle is administered beforethe first rAAV particle. In some embodiments, the second rAAV particle is administered simultaneously with the first rAAV particle. In some embodiments, the first rAAV particle is re-administered a second or a third time following administration of the second rAAV particle. In some aspects, methods of re-administration of any of the disclosed vectors, wherein an immune response in the subject is reduced, minimized or eliminated, are contemplated herein.

### Pharmaceutical Compositions and Cells

In some aspects, a composition is provided which comprises an rAAV particle as described herein (e.g., comprising a AAV44.9(E531D) capsid) and optionally a pharmaceutically acceptable carrier, excipient, diluent and/or buffer. In some embodiments, the compositions described herein can be administered to a mammal (or subject) in need of treatment. In some embodiments, the subject has or is suspected of having FA. In some embodiments, the subject has one or more endogenous mutant alleles (e.g., associated with or that cause a disease, disorder or condition of the eye or retina, such as FA).

Formulation of pharmaceutically-acceptable excipients and carrier solutions is well-known to those of skill in the art, as is the development of suitable dosing and treatment regimens for using the particular compositions described herein in a variety of treatment regimens, including e.g., subretinal, intravitreal, parenteral, intravenous, intranasal, intra-articular, and intramuscular administration and formulation.

Typically, these formulations may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver an rAAV particle as described herein (e.g., comprising a P2-V1 capsid or a variant thereof) in suitably formulated pharmaceutical compositions disclosed herein either subretinally, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of compositions (e.g., comprising an rAAV particle as described herein) suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The term “carrier” refers to a diluent, adjuvant, excipient, or vehicle with which the rAAV particle as described herein is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

The compositions of the present disclosure can be delivered to the eye through a variety of routes. They may be delivered intraocularly, by topical application to the eye or by intraocular injection into, for example the vitreous (intravitreal injection) or subretinal (subretinal injection) inter-photoreceptor space. In some embodiments, they are delivered to rod photoreceptor cells. Alternatively, they may be delivered locally by insertion or injection into the tissue surrounding the eye. They may be delivered systemically through an oral route or by subcutaneous, intravenous or intramuscular injection. Alternatively, they may be delivered by means of a catheter or by means of an implant, wherein such an implant is made of a porous, non-porous or gelatinous material, including membranes such as silastic membranes or fibers, biodegradable polymers, or proteinaceous material. They can be administered prior to the onset of the condition, to prevent its occurrence, for example, during surgery on the eye, or immediately after the onset of the pathological condition or during the occurrence of an acute or protracted condition.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions may be prepared by incorporating an rAAV particle as described herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of composition (e.g., comprising an rAAV particle as described herein) and time of administration of such composition will be within the purview of the skilled artisan having benefit of the present teachings. It is likely, however, that the administration of therapeutically-effective amounts of the disclosed compositions may be achieved by a single administration, such as for example, a single injection of sufficient numbers of rAAV particles to provide therapeutic benefit to the patient undergoing such treatment. Alternatively, in some circumstances, it may be desirable to provide multiple, or successive administrations of the composition, either over a relatively short, or a relatively prolonged period of time, as may be determined by the medical practitioner overseeing the administration of such compositions.

In some embodiments, visual acuity can be maintained or restored (e.g., partially or completely) after administering one or more compositions described in this application. In some embodiments, one or more RGCs may be preserved, partially or completely, and/or one or more rod- and/or cone-mediated functions may be restored, partially or completely, after administering one or more compositions described in this application. For example, thinning of the optic nerve may be reversed.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

In some aspects, the disclosure contemplates host cells that comprise a particle that incorporates a P2-V1 capsid or a variant thereof, a nucleic acid encoding a P2-V1 capsid or a variant thereof, or an rAAV particle as described herein. Such host cells include mammalian host cells, with human host cells being preferred, and may be isolated, e.g., in cell or tissue culture. In some embodiments, the host cell is a cell of the eye.

Accordingly, in some aspects, host cells comprising any of the disclosed rAAV vectors or rAAV particles are provided. In various embodiments, the host cells are photoreceptor cells or RGCs. In some embodiments, the host cells are mammalian (e.g., human) photoreceptor cells or RGCs. In some embodiments, the host cells are human RGCs.

Methods of Producing rAAV Particles

Methods for producing and using pseudotyped rAAV particles are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001). Methods of producing rAAV particles and heterologous nucleic acids are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861; and plasmids and kits available from ATCC and Cell Biolabs, Inc.). For example, a plasmid containing the heterologous nucleic acid may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected or permanently integrated into a producer cell line such that the rAAV particle may be packaged and subsequently purified.

In some embodiments, the one or more helper plasmids include a first helper plasmid comprising a rep gene and a cap gene (e.g., encoding a rAAV capsid protein as described herein) and a second helper plasmid comprising a Ela gene, a Elb gene, a E4 gene, a E2a gene, and a VA gene. In some embodiments, the rep gene is a rep gene derived from AAV2 and the cap gene is derived from AAV2 and may include modifications to the gene in order to produce the modified capsid protein described herein. Helper plasmids, and methods of making such plasmids, are known in the art and commercially available (see, e.g., pDM, pDG, pDPlrs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy,Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R.O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO₄-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the heterologous nucleic acid sequence. As a further alternative, in another example HEK293 or BHK cell lines are infected with a HSV containing the heterologous nucleic acid sequence and optionally one or more helper HSVs containing rep and cap ORFs as described herein and the adenoviral VA, E2A (DBP), and E4 genes under the transcriptional control of their native promoters. The HEK293, BHK, or Sf9 cells may then incubated for at least 60 hours to allow for rAAV particle production. The rAAV particles can then be purified using any method known the art or described herein, e.g., by iodixanol step gradient, CsCl gradient, chromatography, polyethylene glycol (PEG) precipitation, and/or affinity capture.

In various embodiments, an iodixanol step gradient purification method is used. Vectors may be packaged into mammalian cells (e.g., HEK293T cells) and purified by iodixanol gradient centrifugation, followed by buffer exchange and concentration into BSS/Tween buffer. An affinity capture step may be added.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus may be considered to constitute preferred modes for its practice. However, those of skill in the art should, in light of the present disclosure, appreciate that many changes may be made in the specific embodiments which are disclosed and still obtain a like or similar result without departing from the spirit and scope of the invention.

### Example 1

Frataxin is a protein that functions in the mitochondria, which are found throughout the length of neurons. It is proposed that for an FA gene therapy to be effective, the frataxin payload must biodistribute to mitochondria in peripheral axons following administration. To accomplish this, a frataxin gene (or heterologous nucleic acid) containing axon targeting motifs (ATM) was engineered. These ATMs are small cis-acting elements derived from sequences identified to be involved in axonal mRNA localization. These include 3′ UTR sequences derived from, for example, any of β-Actin (β-Act), Synapsin (Syn1), and β-2B tubulin (β-2B Tub), as well as sequence conforming to the small non-coding RNA Y3. These sequences were positioned in the 3′ UTR in AAV constructs with the chimeric CMV/chicken beta actin promoter (CBA) or truncated CBA promoter (smCBA) or Synapsin promoter (Syn1) or Desmin promoter (Des) driving human frataxin open reading frame. FIG. 5 provides an example of an AAV construct containing promoter (in this case synapsin), human frataxin, and an axon targeting motif. Exemplary constructs may contain one or more ATMs positioned 3′ of the human FXN coding region.

A first aim was to establish proof of concept that AAV-FXN preserves retinal structure and function in a conditional knockout mouse model of FA. Studying the impact of FXN mutations on individual organs is challenging due to the system-wide complications associated with this disease. For this reason, conditional knockout mouse lines were recently created that exhibit FXN deficiency only in the heart, or only in proprioceptive (mechanosensory) neurons. Both models allowed investigators to evaluate both the disease and the effects of therapy in specific tissues. By performing selective mouse breeding, a mouse model that lacks FXN expression in the retina only was generated. This allowed for the impact of FA specifically on retina to be investigated, in the absence of complications from other organ systems and, following treatment with AAV-FXN, establishing a proof of concept that gene replacement can preserve/restore retinal structure and function.

To verify the proof of concept model, the mouse homeodomain transcription factor Rx gene (mRx) was used. The Rx gene is one of the earliest genes expressed in the retinal lineage. It is activated between embryonic day (E) 7.5 and E8.0 in the anterior neural plate and later is strongly expressed in the optic vesicles and ventral forebrain. Rx function is essential for vertebrate eye development. Loss of Rx function results in loss of eyes in various vertebrate species, suggesting its conserved role in the eye development. The early onset of Rx expression in the retinal primordium suggested that this locus could be used for driving Cre expression at very early stages of retinal development. It was previously shown that mRx-Cre mouse represents an ideal tool for gene manipulation in early retinal progenitors as judged by its specificity and strength. mRx-Cre mice were re-established on the C57BL6J background, then crossed to Fxn+/− mice (#028040 at Jackson Laboratories). From that breeding, 25% of pups were the desired genotype-mRx-Cre+/−:Fxn+/−. Next, mRx-Cre+/−:Fxn+/− mice were crossed with the floxed Fxn mouse (flFxn+:flFxn+) (#028520 at Jackson Laboratories). From that breeding, 25% of litters are experimental mice-flFxn+Fxn−:Cre+/−, also known as mRx-FXN KO mice.

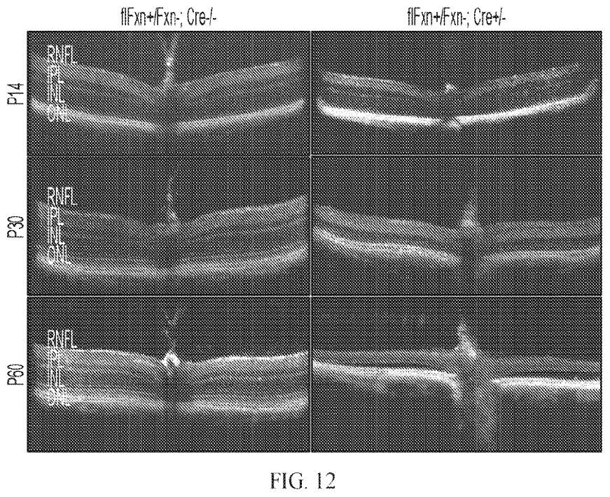

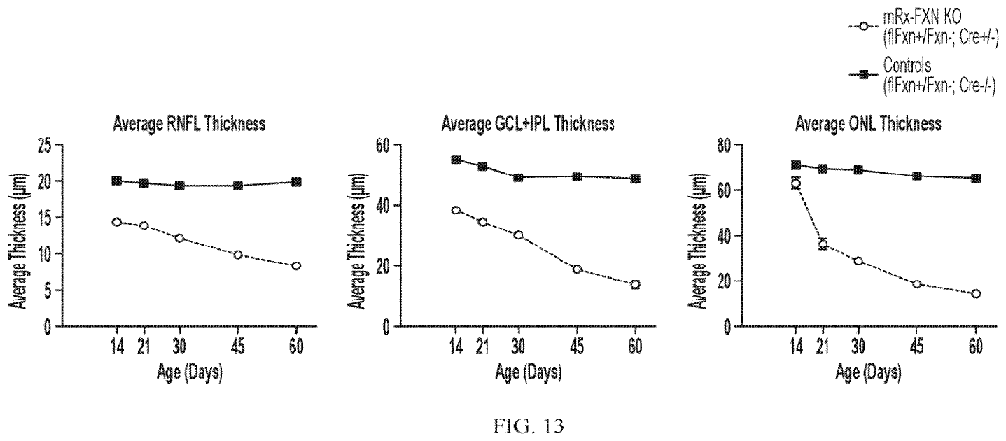

flFxn+/Fxn−; Cre+/− and age-matched flFxn+/Fxn−; Cre−/− controls were assessed with optical coherence tomography (OCT) and electroretinogram (ERG) to evaluate retinal structure and function, respectively, at postnatal days 14, 30 and 60. Representative OCT scans from flFxn+/Fxn−; Cre+/− and age-matched flFxn+/Fxn−; Cre−/− mice are shown in FIG. 12. There is partial degeneration of all retinal layers by day 14 (P14) which becomes progressively more severe by day 30 (P30), and even more so by day 60 (P60). Degeneration is seen at the level of the outer nuclear layer (ONL—photoreceptor cells bodies), inner nuclear layer (INL—cell bodies of secondary order neurons), the inner plexiform layer (IPL—synaptic layer between secondary order neurons and ganglion cells), and the retinal nerve fiber layer (RNFL, axons of ganglion cells). The thickness of each of these layers was quantified in the eyes of all mice (5 experimental, 8 controls) at time points P14, P21, P30, P45, and P60 using Diver software on the Bioptigen OCT machine and averaged. This quantification is shown in FIG. 13. This quantitative data supports observations made in the OCT scans shown in FIG. 12.

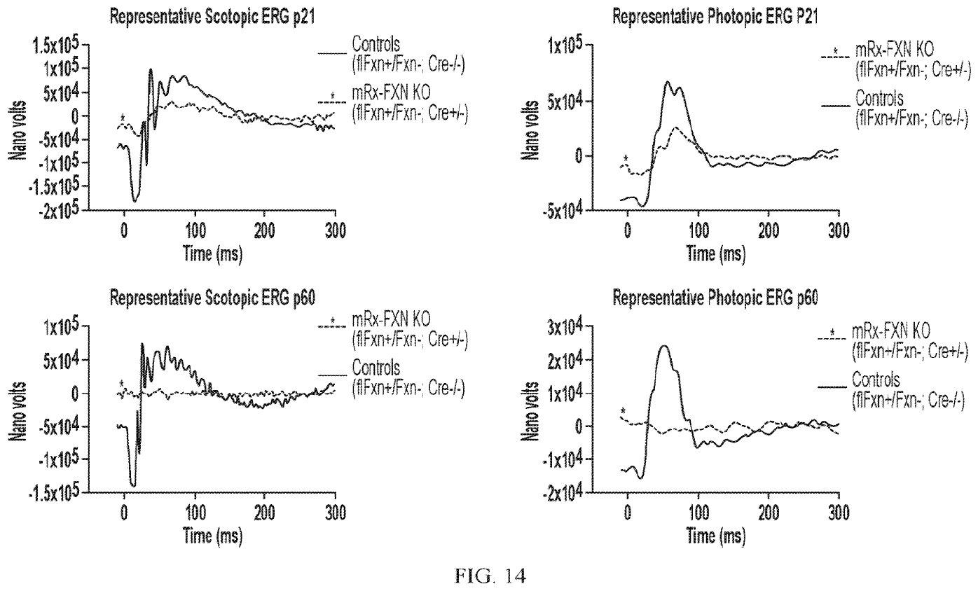

Unsurprisingly, flFxn+/Fxn−; Cre+/− mice also exhibit a progressive loss of retinal function, as observed via electroretinogram (ERG). By P21, rod- and cone-mediated responses in flFxn+/Fxn−; Cre+/− mice were already dramatically reduced. By P60, this loss was more pronounced with only some mice showing small waveforms (FIG. 14). The loss of retinal structure and function in this novel mouse model provided reliable outcome measures against which potential treatments for the visual phenotype of Friedreich's Ataxia can be evaluated.

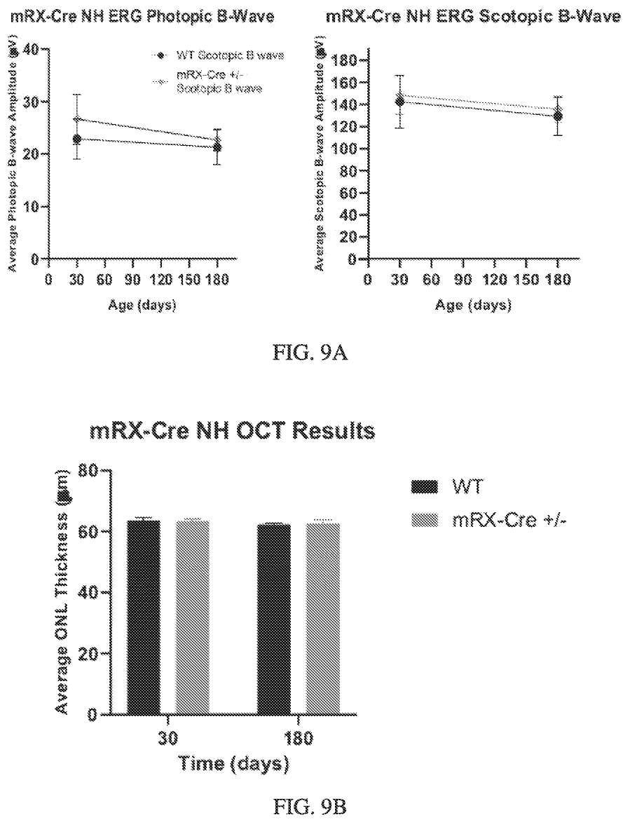

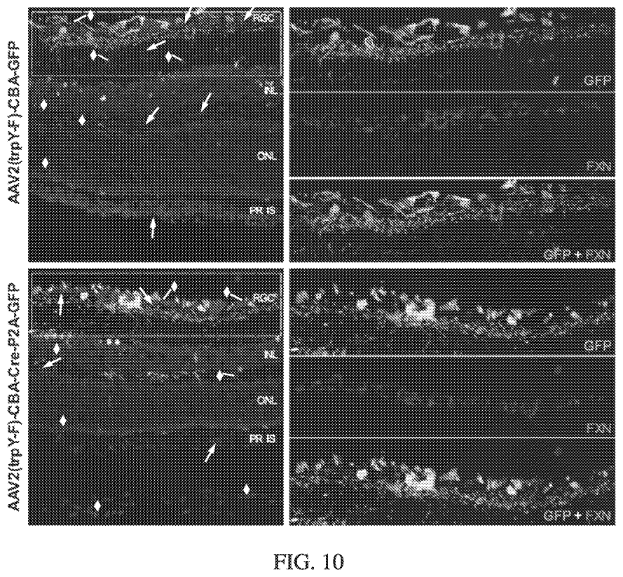

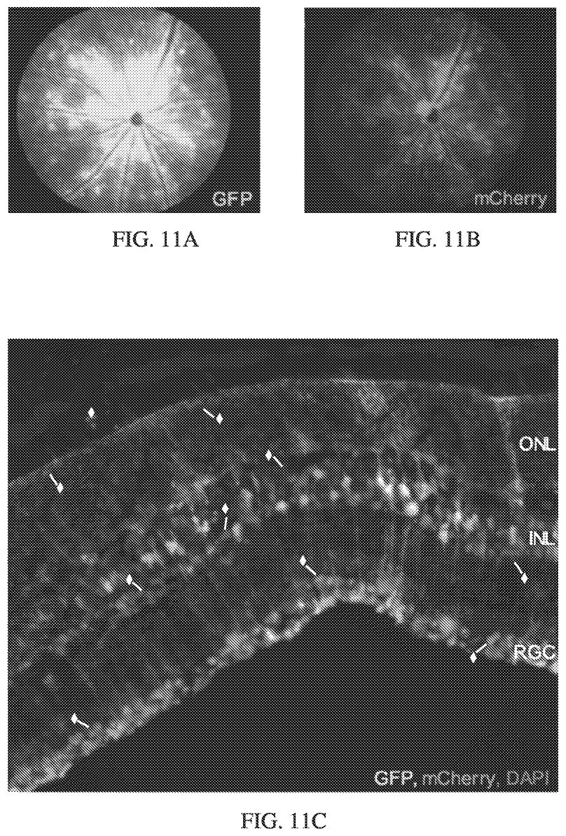

The mRX-Cre mouse line model constitutively expressed Cre in all retinal cell types. Together with heterozygous Fxn knock-out (B6(Cg)-Fxn^{em2.1Lutzy}/J) and Fxn floxed mice C57BL/6J-Fx^{nem2Lutzy}/J mice conditional, retina-only FXN knockout mice (mRx-Fxn KO mice) were generated. An essential natural history study of the mRx-Cre mice was performed as a control expeirment. This was done to ensure that the retina-wide presence of Cre had no negative impact on retinal structure or function that could mask the effects of FXN deficiency in the final model. Indeed, both electroretinogram (ERG) (FIG. 9A) and optical coherence tomography (OCT) (FIG. 9B) revealed no significant difference in retinal function or structure, respectively, between mRx-Cre^{+/-} mice and their WT littermates.

The mRx-Fxn KO mice underwent testing to evaluate the natural history of retinal structure/function and visually guided behavior. In parallel, some cohorts received AAV-FXN gene therapy. At one month of age, mRx-Fxn KO mice received intravitreal injections of AAV-FXN vectors. Two vectors were tested: 1) lead vector P2-V1(Y-F+T-V)-Syn-FXN, and 2) AAV9-CBA-FXN, a vector currently being considered for systemic delivery in FA clinical trials. AAV-FXN is injected in one eye only at either a high (1×10¹² vg/ml) or low (1×10¹¹ vg/ml) dose. To characterize disease progression (natural history) and any potential therapeutic effect in contralateral eyes, mice with contralateral uninjected eyes and unicoular mock injections (vehicle only) is compared. The AAV capsid P2-V1(Y-F+T-V) was chosen for its ability to target retinal ganglion cells (RGCs) and avoid neutralization by AAV2 NAbs. The synapsin (Syn) promoter was chosen to restrict transgene expression to retinal neurons/reduce off-target effects of expression in non-neuronal cells. It was anticipated that AAV9-CBA-FXN will underperform in retina relative to the lead vector, but it was included in this testing since it is already under clinical consideration for treating FA.

Naive littermates are also included in the natural history study. In all mice, retinal structure are evaluated monthly until at least 1 year of age using fundoscopy and optical coherence tomography (OCT). Emphasis is placed on the appearance of the optic nerve head and the thickness of the nerve fiber layer, both of which are indicative of RGC degeneration. The thickness of the outer nuclear layer (ONL) is also quantified to determine whether FXN deficiency impacts photoreceptors. High resolution three-dimensional MRI is used to assess thickness of the optic nerve. Retinal function is evaluated monthly for at least 1 year with electroretinogram (ERG). Both scotopic, photopic, and pattern ERG recordings are performed to isolate the function of rods, cones, and retinal ganglion cells (RGCs), respectively. After the final ERG, optokinetic reflex testing (visually guided behavior) is performed. All aforementioned outcome measures are performed in life. At one year, mice are sacrificed and their retinas evaluated for the presence of AAV-mediated FXN via immunohistochemistry of frozen retinal sections and western blot. qPCR is used to evaluate levels of AAVmediated FXN transcript. Retinal wholemounts are stained with anti-beta-tubulin III (TUJ1) antibody and microscopy used to quantify the number of RGCs in treated vs. untreated eyes. Histopathology is performed on longitudinal sections of the optic nerve to evaluate optic atrophy/axonal loss. In the event that a retinal phenotype has not emerged by 1 year of age, the mice are allowed to remain alive for an additional six months and monitored as above. The aforementioned study will inform whether the loss of retinal structure/function associated with FA can be prevented. Once the age at which a retinal phenotype manifests is established, the approach will be refined to evaluate whether AAV-FXN gene therapy can arrest or reverse disease progression after onset. mRx-Fxn KO mice are intravitreally injected with the identical vector above after onset and monitored monthly thereafter as described above.

Further studies aim to establish manufacturing of clinical candidate AAV-FXN vector, package virus suitable for GLP safety studies, and establish safety in non-human primate. Differences in retinal anatomy and biochemistry dictate that gene therapies showing proof of concept in small animal models undergo careful consideration prior to clinical application. If necessary, they are modified based on results of testing in primate retina. Macaques have a foveated retina of similar scale and cellular distribution to humans.

Upstream materials required for large scale AAV manufacturing will be created and validated. The optimal AAV-FXN vector identified above will be manufactured at large scale. Vectors will be purified using methods scalable and amenable to GMP manufacturing and will be tested to ensure compliance with specifications established for GLP test article (qualified titer assay, capsid purity, endotoxin, bioburden). Prior to purchase, macaques will be screened for the presence of serum neutralizing antibodies (NAbs) against AAV2 or AAV9. Macaques will receive intravitreal injections of AAV-FXN(HA). The HA tag is included to distinguish vector mediated FXN from endogenous signal. Right eyes will receive a high dose (1×10¹¹ vg/ml), and left eyes will receive a low dose (1×10¹⁰ vg/ml) of vector.

General ophthalmological exams will be performed before and after injection to evaluate whether injections were well tolerated. In life imaging (fluorescent Spectralis and OCT scans) will be performed and 4, 8 and 12 weeks post-injection to evaluate retinal structure. Serum will be collected at the time of second imaging and analyzed for the presence of NAbs. NHPs will be sacrificed at 12 weeks post-injection. Retinas from the right and left eyes of one animal will be used to measure levels of AAV-mediated FXN expression using qPCR. Eyes from the other two animals will be enucleated, fixed overnight, their retinas blocked, cryoprotected and sectioned. Retinas will be stained with antibodies specific for HA, RGCs, and photoreceptors and counterstained with DAPI. Stained retinas will be visualized with confocal microscopy. Optic nerves, brains and systemic tissues will be collected from all animals for biodistribution analysis. If it is determined that intravitreal injection (IVI) of the optimal capsid inefficiently transduces RGCs, subILM delivery of the same capsid using previously published methods will be explored (see Boye et al., Human Gene Ther. 2016; 27:580-97).

### Example 2

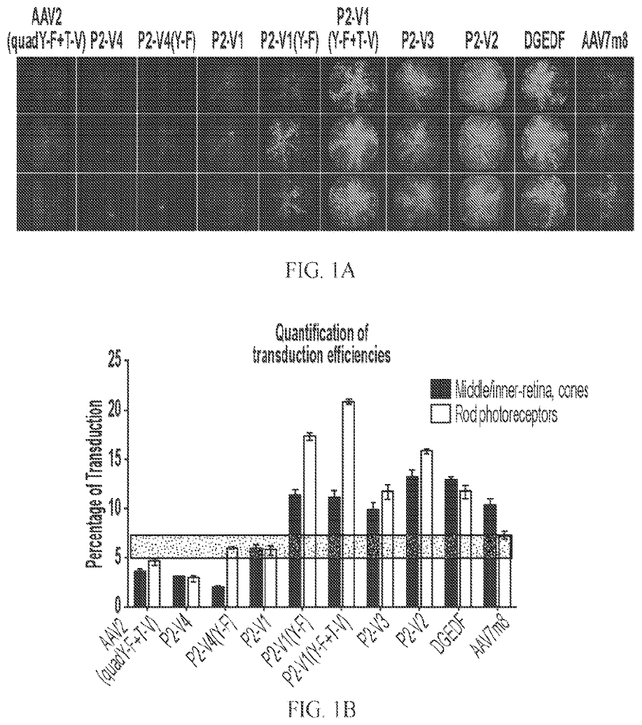

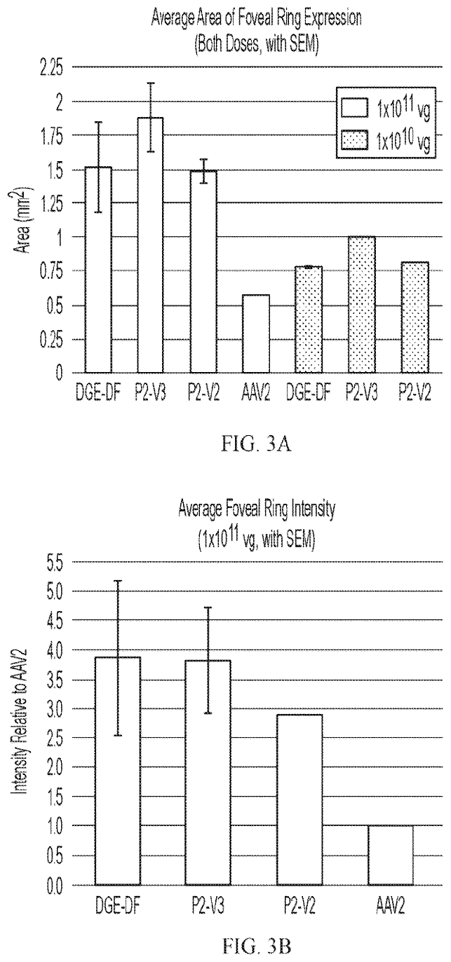

A method was previously developed to create macaques with sortable photoreceptors (PRs) and RGCs (see US Publication No. 2020/0330611 and Choudhury et al., Front Neurosci. 2016; 10:551). RGCs is cell type most impacted in FA phenotypes. First, macaques underwent bilateral, subretinal injection of AAV5-GRK1-GFP, a capsid and promoter combination known to drive GFP expression exclusively in rods and cones. Five blebs (40-100 μL) were delivered, including one under the macula. Next, RGCs were made sortable via injections of a dye into the lateral geniculate nuclei which retrogradely labels these cells. This allowed for screening of a highly complex AAV2-based capsid library and identification of variants enriched in both PRs, and RGCs. These include P2-V1, P2-V1(Y-F+T-V), P2-V2, P2-V3, DGE-DF, and P2-V4. Enriched capsids were vectorized and most confirmed to exhibit enhanced retinal transduction following Ivt injection in mice relative to benchmark capsids, AAV2(7m8) and AAV2(quadY-F+T-V) (FIGS. 1A-1B). The addition to these variants of amino acid substitutions known to be implicated in proteosomal avoidance further increased their potency. A select few were tested in intravitreally-injected macaques, and confirmed to have higher potency than AAV2. These include DGE-DF, P2-V2 and P2-V3 (FIG. 3).

A major hurdle faced by intravitreally delivered AAVs is potential neutralization in the vitreous. AAV2-based vectors are ideal for targeting retina via the vitreous but approximately 70% of the human population has been exposed to AAV2 and thus harbor neutralizing antibodies against this capsid, which could result in only 30% of FA patients benefitting from treatment. P2-V1 was the most enriched variant in the primate library screen, yet this capsid did not exhibit enhanced potency relative to benchmark vectors in intravitreally injected mice.

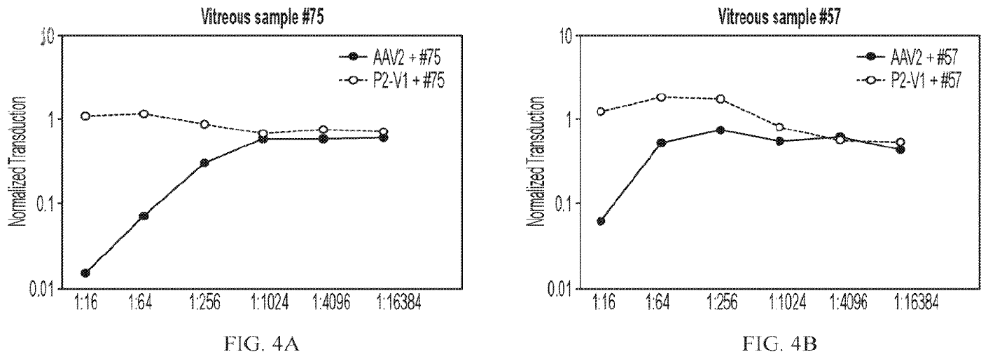

It was hypothesized that the enrichment of P2-V1 was driven by a different selective pressure, e.g., an increased ability to avoid neutralization by pre-existing neutralizing antibodies (NAbs) against AAV. It was reasoned that this selective pressure was added to the capsid library screen by virtue. Hundreds of human vitreous samples from patients undergoing vitreoretinal surgery and screened them for the presence of AAV2 Nabs were collected. Using individual human vitreous samples identified to harbor a moderate to high titer of neutralizing antibodies against AAV2 (approximately 25% of samples), the ability of P2-V1 to avoid neutralization compared to AAV2 was assessed. Experiments were performed such that the theoretical vector: vitreous volume was similar to that in an IVI macaque/human (assumed a typical dose of AAV in 100 μL of 1×10¹¹ to 10¹² vgs/mL). P2-V1-mCherry or AAV2-mCherry vectors were pre-incubated with three separate inhibiting vitreous samples (#56, #57, and #75) at a series of dilutions, as well as a control, non-AAV2 neutralizing vitreous sample (#104). ARPE19 cells (a line of human RPE cells) were then infected with each sample at an MOI of 5000. Three days post-infection (p.i.), cells were dissociated and mCherry expression quantified by flow cytometry. Samples #56, #57, and #75 strongly neutralized AAV2. However, samples #75 and #57 failed to neutralize P2-V1 (FIG. 4). Neither AAV2 nor P2-V1 were neutralized by sample #104 (data not shown). The results confirm that screening of ‘CapLib7’ generated an ‘immune avoidance’ variant P2-V1 that could immediately impact designs of IVI gene therapy trials. The P2-V1 has continued to be evaluated head to head against AAV2. At last count, P2-V1 outperformed AAV2 in 72% (21 of 29) of human vitreous samples harboring NAb. This analysis has been extended to a P2-V1-based variant that has undergone additional rational design, resulting in higher potency in mouse-P2-V1(Y-F+T-V) (FIG. 1). Indeed, P2-V1(Y-F+T-V) maintained the parent capsid's ability to avoid NAbs in 2 out of 2 inhibiting vitreous samples tested thus far (data not shown). The P2-V1 has also been compared to AAV2(7m8). P2-V1 and outperforms AAV2(7m8) in 16 out of 22 (73%) inhibiting human vitreous samples.

Notably, due to the method by which sortable cells were created in macaques for library screening (subretinal injection of an AAV5 vector), the neutralizing antibodies (Nabs) mounted by the NHPs in the screen were to AAV5, which phylogenetically (and at the level of the capsid) is very dissimilar to AAV2. Hence, selected variants may show avoidance of Nabs to capsids that are within the spectrum of AAV5 and AAV2. With these novel vectors, the number of patients amenable to treatment will increase to >30%. This would be true for advanced stage FA patients who are older/more likely to harbor pre-existing AAV2 NAbs, and patients that were treated previously with a systemic AAV therapy. Incorporation of rAAV-FXN into these novel ‘NAb avoidance’ capsids may preserve RGC function/structure, and vision in FA patients.

### Experimental Results:

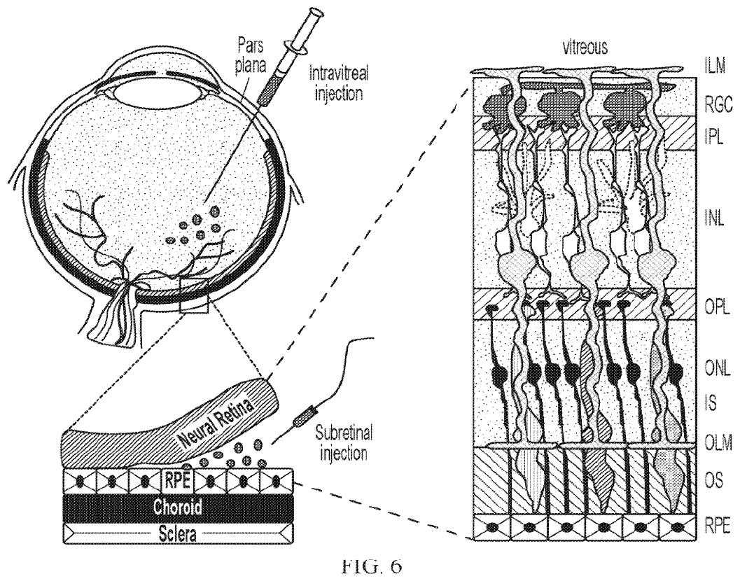

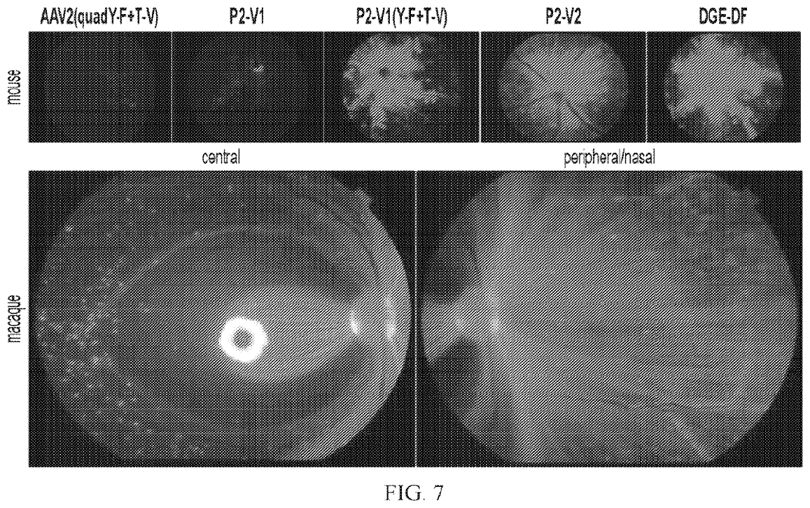

Intravitreal injection (IVI) is a promising delivery route for targeting RGCs in the inner retina, but requires an AAV capsid capable of ‘penetrating’ through inner limiting membrane (a typical basement membrane secreted by the endfeet of Muller glia that forms the vitreo-retinal interface). See FIG. 6. IVI is attractive because it can be performed in clinic rather than a surgical suite, thereby increasing accessibility to larger patient populations. However, the limited clinical data, and non-human primate (NHP) studies utilizing currently available capsids indicate that the barriers to IVI AAV (i.e., dilution/neutralization in vitreous, inner limiting membrane) and pre-existing immunity to AAV may restrict gene expression to levels that are ‘subtherapeutic’ in a substantial proportion of patients. Currently patients with measurable levels of pre-existing neutralizing antibodies (NAbs) are often excluded from treatment altogether. Researchers have worked for years to develop novel AAV capsids capable of overcoming these challenges. A complex, structurally informed, AAV2-based capsid library (“CapLib7”) with several advantages over those previously described5 in macaque, a model organism with ocular characteristics most similar to man was screened. CapLib7 was built on an AAV2 backbone, ensuring maintenance of the required capsid biology for IVI mediated transduction (e.g., HSPG binding), and designed using structural alignment of the 9 variable regions (VR) of capsid in all known parvoviruses. For this screening to be possible, first a method was developed for fluorescently labelling target cells in macaque retina from which vector DNA could be isolated after sorting with FACS. Rods and cones were specifically labeled by subretinal injection (SRI) of AAV5-GRK1-GFP, and RGCs were retro-labeled via MRI-guided injections of LGN with rhodamine dextran (microruby). The recovery of distinct cell populations was confimred and their purity was validated via qPCR7. In round 1, a macaque underwent bilateral, SRI of AAV5-GRK1-GFP. Five blebs (40-100 μL) were delivered, including one under the macula. LGN injection of microruby occurred 25 days later, followed by IVI of 120 μl of ‘CapLib7’ per eye at day 27. Following separate recovery from PRs and RGCs, two sublibraries (PR and RGC) were regenerated for round 2. Each sub-library was separately injected into 2 animals each (animals receiving PR sublibrary were pre-labeled with SRI AAV5-GRK1-GFP, and animals receiving RGC sub-library were retro-labeled by LGN injection of microruby). Titers of the original and subsequent sub-libraries ranged between 5×10¹¹ vg/ml and 3×10¹² vg/ml. RPE was collected by manual separation after round 2.

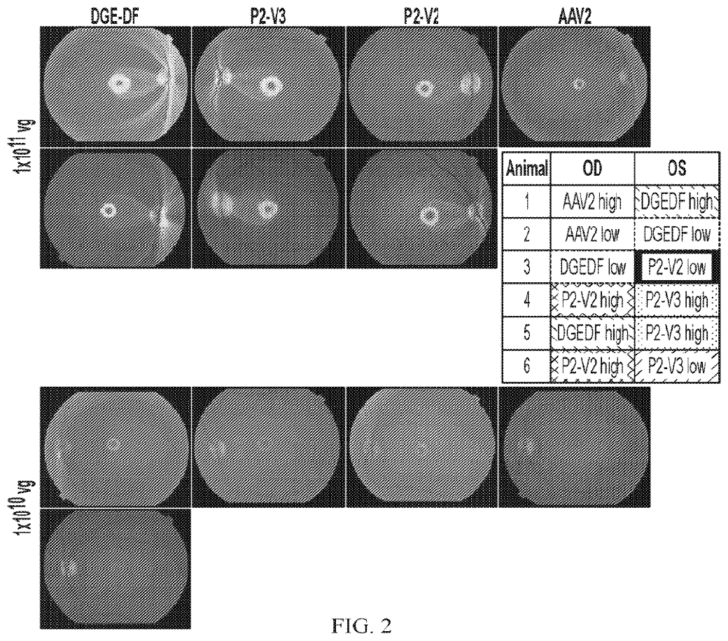

Amplicons arising from round 2 in the primate screen underwent PacBio sequencing to evaluate diversity. All variants displayed improved transduction in vitro relative to AAV2. All but one exhibited enhanced transduction in IVI Nri-GFP mice relative to the benchmark vector, AAV2(quadY-F+T-V). A handful of these novel variants are shown in FIG. 7. It has since been shown that novel variants, when individually IVI in macaque, exhibit increased potency relative to AAV2 (FIG. 2).

P2-V1 was the most enriched variant after 2 rounds of selection in the primate screen. When tested by IVI in mice, transduction by P2-V1 was equal to the top benchmark vector AAV2(quadY-F+T-V), yet there were other variants identified in the screen with higher transduction efficiencies (FIG. 7). This observation and the limited number of primates available for testing is the reason P2-V1 was not included in the experiment assessing transduction in macaque retina of individual variants (FIG. 2). It was reasoned that enrichment of P2-V1 must be driven by an additional selective pressure. Primates used in the capsid library selection experiments were pre-screened for the presence of anti-AAV2 neutralizing antibodies (NAbs), with all animals having <10 NAb50 (reciprocal of the dilution at which 50% neutralization is observed). However the subretinal injections of AAV5-GFP performed to create sortable photoreceptors (multiple blebs, with variable levels of leakage into the vitreous) resulted in the production of anti-AAV5 NAbs that were present at the time of capsid library delivery (confirmed in serum collected on day of library dosing, data not shown). It is well established that anti-AAV antibodies display crossreactivity, including antibodies generated against ocularly administered AAV5 cross-reacting with AAV23. An analysis of substitutions found in P2-V1 relative to AAV2 indicated that three of the sixteen were previously individually identified to confer antibody avoidance when early generation AAV2 libraries were screened in vitro against 1) pooled human Ig strongly neutralizing to AAV2, 2) polyclonal serum from rabbits immunized with AAV2, or 3) monoclonal Ab (A20) against AAV29′11. It was hypothesized that avoidance of neutralization by anti-AAV antibodies was a contributing factor in the enrichment of P2-V1 in the IVI primate capsid library screen.

Human vitreous samples from patients undergoing vitreoretinal surgery were collected and screened for the presence of NAb to AAV2. The parameters of the screening assay models the vector titer and vitreous volume that exists when a clinically relevant dose of AAV (1×10¹⁰ to 1×10¹¹ vg) is IVI in a human or macaque eye and conforms to what is currently being evaluated in clinical trials. Using this approach, it was discovered that approximately 25% of human vitreous samples contained moderate to high levels of anti-AAV2 NAbs (>90% reduction in transduction at a 1:4 dilution of vitreous at a 5000 MOI). The ability of P2-V1 to avoid neutralization by the AAV NAbs in these ‘inhibiting’ human vitreous samples relative to AAV2 was assessed. P2-V1-mCherry or AAV2-mCherry vectors were preincubated with inhibiting vitreous samples at a series of dilutions. ARPE19 cells were then infected with each sample at an MOI of 5000. Three days post-infection (p.i.), cells were dissociated and mCherry expression quantified by flow cytometry. Samples #56, #57, and #75 strongly neutralized AAV2. However, samples #75 and #57 failed to neutralize P2-V1 (FIG. 4). Neither AAV2 nor P2-V1 were neutralized by a control vitreous sample previously found to be free of AAV NAbs (data not shown). At last count, P2-V1 has outperformed AAV2 in 76% (16 of 21) of human vitreous samples harboring NAb. This analysis was extended to a P2-V1-based variant that has undergone additional rational design, resulting in higher potency in mouse-P2-V1(Y-F+T-V) (FIG. 6). Indeed, P2-V1(Y-F+T-V) maintained the parent capsid's ability to avoid NAbs in 2 out of 2 inhibiting vitreous samples tested thus far (data not shown). The results confirm that screening of ‘CapLib7’ generated a “neutralizing antibody avoidance” variant, P2-V1, that could immediately impact designs of IVI-based gene therapy trials. P2-V1 has continued to be evaluated head-to-head against AAV2.

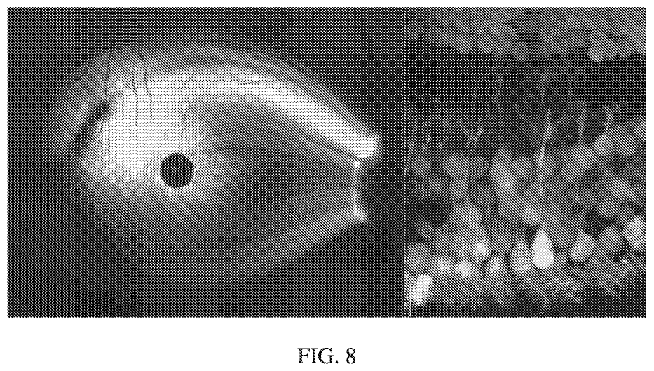

SubILM injection is an alternative surgical approach for efficiently targeting RGCs in the inner retina. The effect of physical circumvention of the ILM on alteration of the magnitude and pattern of retinal transduction relative to that seen with IVI AAV2 in macaque has been explored. To do so, a surgically induced, hydrodissected space between the ILM and neural retina (i.e., subILM) was created where the vector was sequestered. The goal was to bypass three major barriers associated with intravitreal injection-dilution of vector in the vitreous humor, exposure to neutralizing antibodies and the ILM itself. Surgical manipulation of the ILM is routinely performed in humans to correct macular holes, macular puckers and myopic foveoschisis. The ILM can be surgical minimized (i.e., ‘peeled’) and AAV immediately delivered to the vitreous, but this leaves the neural retina exposed to the immunological environment of the vitreous, which has just been exposed to antigen (AAV capsid). Such an approach can lead to inflammation in NHP retina. For this reason, subILM deliveryof AAV in macaque as a possible alternative to IVI (FIG. 8) was pursued. subILM-injected AAV2 transduced almost all RGCs within the bleb, Muller glia, sporadic ON bipolar cells (ON BCs) and cones. The efficiency and depth of transduction was greatly enhanced relative to IVI vector. There is confidence that the use subILM injection (paired with these more potent capsids) could be used as an alternative approach in the event that higher transduction efficiency (above that achievable with IVI of the novel capsid variants) is required.

### Example 3

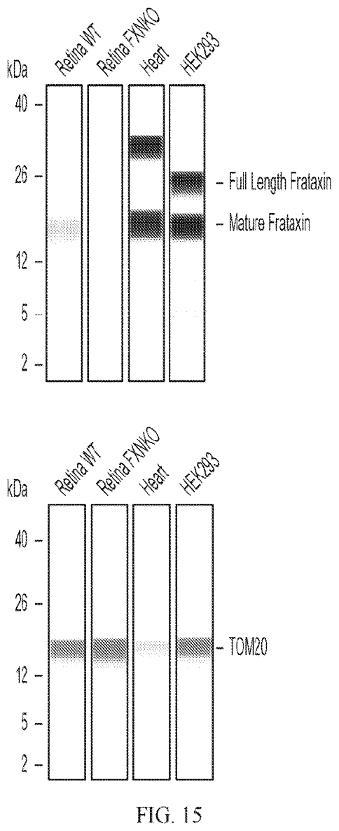

A protocol for detecting FXN expression in mouse retina using the capillary-based Protein Simple Jess quantification system was optimized. Full knockout of FXN in retinas of mRx-Fxn KO mice, and the ability to detect endogenous FXN in retinas and hearts of wild type mice, as well as HEK293 cells transfected with FXN plasmid was confirmed (FIG. 15). Detection was performed with mouse anti-frataxin antibody clone 4f9 from EMD Millipore, (catalog number: MABN2313-100UG) at a 1:1000 dilution. In WT mouse retina, only mature FXN was detected. In the heart, mature FXN and one background band that likely stems from contaminating signal in the blood was detected. In HEK293 cells transfected with FXN plasmid, both full length and mature FXN were detected. Mitochondrial protein, TOM20 was used as the loading control and was detected with rabbit anti-TOM20 D8T4N #42406S at a 1:100 dilution.

An FXN construct for delivery via AAV was optimized (based on NCBI sequence NP_000135.2, human frataxin, mitochondrial isoform 1 preproprotein). A Kozak sequence was added in addition to NotI sites on both ends (for ease of cloning in and out of other constructs) An internal NotI site was eliminated and an HA tag (which will ultimately be used for detection in wild-type macaque models that express endogenous FXN) was added. This construct was cloned downstream of either the ubiquitous CBA (chicken beta actin) promoter, or the neuronal-specific SYN (synapsin) promoter. The latter is intended to restrict FXN expression to retinal neurons and mitigate any potential inflammation. These optimized constructs (with and without HA tag) were packaged in P2-V1(Y-F+T-V), the optimal AAV capsid for intravitreal delivery.

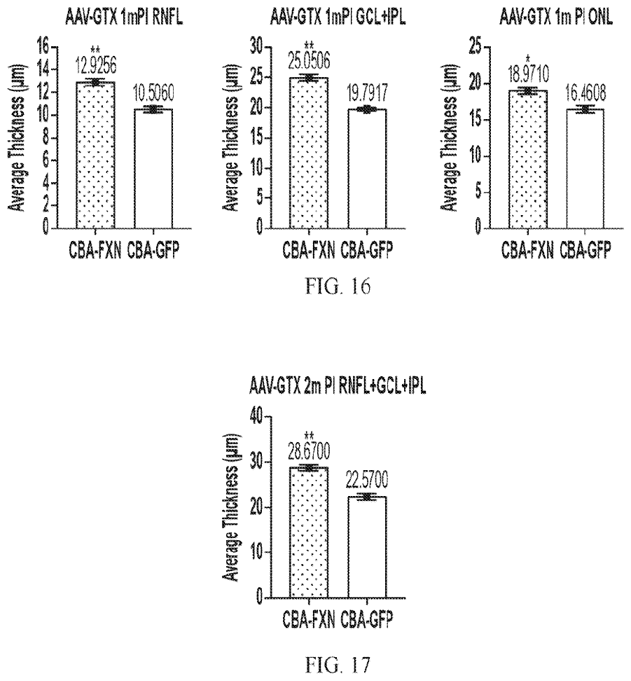

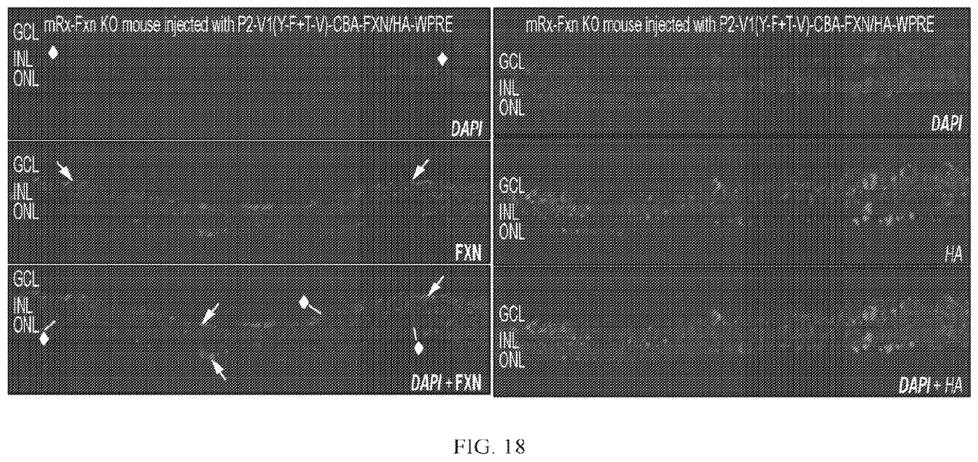

Preliminary results show that intravitreal injection of AAV-FXN in mRx-FXN KO mice significantly preserves retinal structure. The impact of AAV-mediated FXN replacement in the novel mRx-FXN KO mice was evaluated. The complex breeding scheme provided a few mRx-FXN KO mice in each litter. As such, six mice were treated. Each mouse was intravitreally injected in their right eye with P2-V1(Y-F+T-V)-CBA-FXN/HA-WPRE and in their left eye with control vector, P2-V1(Y-F+T-V)-CBA-GFP. Vectors were delivered at 2×10¹² vg/mL (2×10⁹ vg delivered in 1 μL) at day 15 (P15). At 1 month post-injection (P30), the thickness of the RNFL, GCL+IPL (ganglion cell layer and inner plexiform layer), and ONL layers in AAV-FXN or AAV-GFP treated eyes (FIG. 16) was quantified. OCT analysis revealed significant preservation of all retinal layers in eyes treated with AAV-FXN relative to that seen in AAV-GFP controls. Protection of the outer retina layer (ONL) in AAV-FXN-treated eyes was significant but less pronounced than that seen in inner retinal layers. This is consistent with the notion that intravitreally-delivered AAV vectors more efficiently transduce cells within the inner vs. the outer retina. By two months post-injection, delineation of the retinal layers became difficult. As such, the total retinal thickness between AAV-FXN and AAV-GFP injected eyes was compared. Significant preservation of retinal structure was still evident at this time point (FIG. 17).

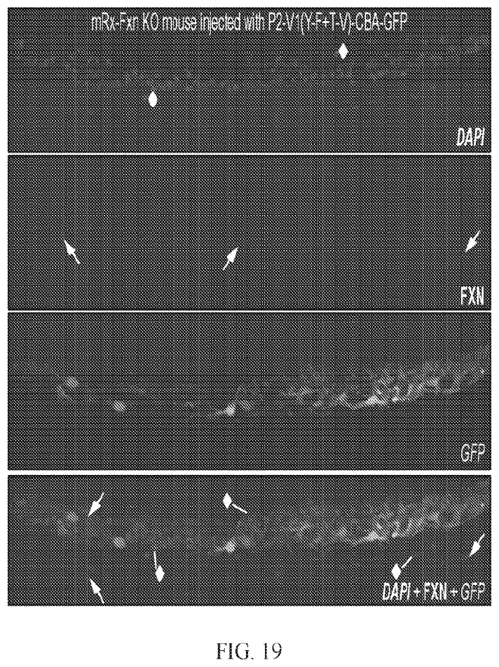

AAV-mediated FXN/HA was detected in the retinas of intravitreally injected mRx-FXN KO mice. At 2 months post-injection, mice were sacrificed and evaluated for the presence of AAV-mediated FXN and HA expression. AAV-mediated FXN and HA expression was present mainly within the retinal ganglion cells and residual photoreceptor cell bodies of treated mice (FIG. 18). In control eyes, there was very low (if any) FXN signal (FIG. 19). Degeneration in control eyes had progressed to the point of an inability to distinguish between retinal cell layers. AAV-mediated GFP expression was present in a fraction of remaining cells.

### Other Embodiments

It should be understood that the examples and embodiments described herein are for illustrative purposes only and that various modifications or changes in light thereof may be suggested to persons skilled in the art and are to be included within the spirit and purview of this application and the scope of the appended claims. Recitation of ranges of values herein are merely intended to serve as a shorthand method of referring individually to each separate value falling within the range, unless otherwise indicated herein, and each separate value is incorporated into the specification as if it were individually recited herein. The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein may be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method comprising administering to one or both eyes of a mammalian subject a recombinant AAV (rAAV) particle comprising an rAAV vector that comprises a heterologous nucleic acid encoding human frataxin (FXN).

2. The method of claim 1, wherein the rAAV vector further comprises a 3′ untranslated region (UTR) comprising one or more axon targeting motifs (ATMs).

3. The method of claim 1 or 2, wherein the subject is human.

4. The method of any one of claims 1-3, wherein the particle is administered by intravitreal, subretinal, or subILM injection.

5. The method of any one of claims 1-4, wherein the particle is administered by intravitreal injection.

6. The method of any one of claims 1-5, wherein the step of administering a) preserves one or more retinal ganglion cells (RGCs), b) restores nerve fiber layer or optic nerve head integrity, c) restores one or more rod- and/or cone-mediated functions, or d) restores completely or partially visual behavior in the one or both eyes; or any combination thereof.

7. The method of any one of claims 1-6, wherein the step of administering reverses visual loss or optic nerve atrophy in the subject.

8. The method of any one of claims 1-7, wherein the rAAV vector comprises a sequence having at least 85% identity, at least 90% identity, at least 92.5% identity, at least 95% identity, 98% identity, or 99% identity to the nucleotide sequence of any one of SEQ ID NOS: 8, 18, and 24-27.

9. The method of any one of claims 1-8, wherein the rAAV comprises the nucleotide sequence of any one of SEQ ID NOS: 8, 18, and 24-27.

10. The method of any one of claims 1-9 further comprising administering intravenously, intrathecally, or intracisternally to the subject a second rAAV particle comprising a second rAAV vector that comprises a human FXN coding sequence.

11. The method of claim 10, wherein the second rAAV particle is administered after the first rAAV particle.

12. The method of claim 10, wherein the second rAAV particle is administered before, or simultaneously with, the first rAAV particle.

13. A recombinant AAV (rAAV) vector comprising a polynucleotide that comprises a heterologous nucleic acid encoding human frataxin (FXN), and a 3′ UTR that comprises one or more ATMs.

14. The rAAV vector of claim 13, wherein the one or more ATMs comprise sequences derived from β-actin, synapsin, β-tubulin, and/or non-coding RNA Y3.

15. The rAAV vector of claim 13 or 14, wherein the heterologous nucleic acid is codon-optimized for expression in human cells.

16. The rAAV vector of any one of claims 13-15, wherein the heterologous nucleic acid comprises one or more silent mutations in the coding region.

17. The rAAV vector of any one of claims 13-16, wherein the polynucleotide comprises a sequence having at least 85% identity, at least 90% identity, at least 92.5% identity, at least 95% identity, 98% identity, or 99% identity to the nucleotide sequence of any one of SEQ ID NOS: 8 and 24-27.

18. The rAAV vector of any one of claims 13-17, wherein the polynucleotide comprises a sequence that differs by 5, 10, 15, 20, 25, or more than 25 nucleotides from the nucleotide sequence of any one of SEQ ID NOS: 8 and 24-27.

19. The rAAV vector of any one of claims 13-18, wherein the polynucleotide comprises the sequence set forth in any one of SEQ ID NOS: 8 and 24-27.

20. The rAAV vector any one of claims 13-19, wherein the polynucleotide comprises a nucleic acid sequence encoding a hemagglutinin (HA) tag.

21. The rAAV vector of any one of claims 13-20, wherein the heterologous nucleic acid is operably linked to one or more regulatory elements that direct expression of the heterologous nucleic acid in a photoreceptor cell or retinal ganglion cell.

22. The rAAV vector of any one of claims 13-21, wherein the polynucleotide comprises a promoter that is capable of expressing the nucleic acid sequence in one or more photoreceptors or RGCs of a mammalian eye.

23. The rAAV vector of claim 22, wherein the promoter is an EF1-α promoter, a human synapsin (hSYN1) promoter, a Nefh(Ple345) promoter, a Sncg promoter, a Cx36 promoter, a dcx promoter, chimeric CMV/CBA promoter, a truncated chimeric CMV/CBA (smCBA) promoter, a CBA hybrid (CBh) promoter, a PGK-1 promoter, or a desmin promoter.

24. The rAAV vector of claim 22 or 23, wherein the promoter is a human synapsin (hSYN1) promoter or an EF1-α promoter.

25. The rAAV vector of any one of claims 13-24, wherein the polynucleotide comprises a WPRE element.

26. The rAAV vector of claim 25, wherein the WPRE element comprises the nucleotide sequence of SEQ ID NO: 15.

27. The rAAV vector of claim 25 or 26, wherein the WPRE element is positioned 3′ of the heterologous nucleic acid.

28. The rAAV vector of any one of claims 13-27, wherein the vector is self-complementary.

29. The rAAV vector of any one of claims 13-28, wherein the polynucleotide is flanked by one or more inverted terminal repeat (ITR) sequences.

30. The rAAV vector of claim 29, wherein the one or more ITR sequences comprises the nucleotide sequence of SEQ ID NO: 29 and 30.

31. The rAAV vector of any one of claims 13-30, wherein the vector comprises a nucleotide sequence having at least 95% identity, 98% identity, or 99% identity to the nucleotide sequence of SEQ ID NO: 16 or 17.

32. The rAAV vector of any one of claims 13-31, wherein the vector comprises the nucleotide sequence of SEQ ID NO: 16 or 17.

33. The rAAV vector of any one of claims 13-32, wherein the heterologous nucleic acid further comprises an inert stuffer to bring the length of the polynucleotide to between 4.5 kB and 4.8 kB.

34. A recombinant adeno-associated viral (rAAV) particle comprising a capsid, wherein the rAAV particle further comprises the rAAV vector of any one of claims 13-33.

35. The rAAV particle of claim 34, wherein the capsid comprises an amino acid sequence having at least 95% sequence identity to any one of SEQ ID NOs: 1, 2, 6 and 32-40.

36. The rAAV particle of claim 34, wherein the capsid comprises any of the following capsid proteins: AAV5 and variants thereof, AAV7 and variants thereof, AAV8 and variants thereof, AAV9 and variants thereof, AAV2(7m8), AAV2(4pMut)ΔHS, AAV44.9, AAV44.9(E531D), AAV44.9(T492V+E531D), AAV44.9(Y446F+E531D), and AAV44.9(Y446F+T492V+E531D), AAV8(Y447F+Y733F+T494V), AAVrh.8, AAVrh.8R, AAVrh.10, AAVrh.74, AAV2TT, AAV2HBKO, and AAVAnc80.

37. The rAAV particle of claim 34, wherein the capsid comprises any of the following capsid protein variants: AAV2(7m8), DGE-DF, P2-V1, P2-V2, P2-V3, P2-V1(Y-F), and P2-V1(Y-F+T-V).

38. The rAAV particle of claim 34, wherein the capsid comprises P2-V1(Y-F+T-V).

39. The rAAV particle of claim 34, wherein the capsid comprises an AAV5 capsid protein, or a variant thereof.

40. A composition comprising a plurality of the rAAV particle of any one of claims 34-38 further comprising one or more pharmaceutically acceptable excipients.

41. A cell comprising the rAAV vector of any one of claims 13-32 or the rAAV particle of any one of claims 34-39.

42. The cell of claim 41, wherein the cell is a mammalian photoreceptor cell or RGC.

43. A method for transducing a mammalian photoreceptor cell or RGC, the method comprising administering to one or both eyes of a mammal the rAAV particle of any one of claims 34-39 or the composition of claim 40.

44. A method for treating or ameliorating Friedreich's ataxia in a mammal, the method comprising administering to one or both eyes of the mammal the rAAV particle of any one of claims 34-39 or the composition of claim 40 in an amount sufficient to treat or ameliorate one or more symptoms of the ataxia in the mammal.

45. The method of claim 44, wherein the administration is intravitreal.

46. The method of claim 44 or 45, wherein the mammal is human.

47. The method of any one of claims 44-46, wherein the step of administering a) preserves one or more RGCs, b) restores nerve fiber layer or optic nerve head integrity, c) restores one or more rod- and/or cone-mediated functions, or d) restores completely or partially visual behavior in the one or both eyes; or any combination thereof.

48. The method of any one of claims 44-47, wherein the step of administering reverses visual loss or optic nerve atrophy in the subject.

49. The method of any one of claims 44-48 further comprising administering intravenously, intrathecally, or intracisternally to the subject a second rAAV particle comprising an rAAV vector that comprises a human FXN coding sequence.

50. The method of claim 49, wherein the second rAAV particle is administered after the first rAAV particle.

51. The method of claim 49, wherein the second rAAV particle is administered before, or simultaneously with, the first rAAV particle.
